(19) United States
(12) Patent Application Publication
(10) Pub. No.: US 2025/0205366 A1
(43) Pub. Date: Jun. 26, 2025

(54) GUIDE NUCLEIC ACID TARGETING MECP2 AND USES THEREOF

(71) Applicants: HuidaGene Therapeutics (Singapore) Pte. Ltd., Singapore (SG); Center for Excellence In Brain Science And Intelligence Technology, Chinese Academy of Sciences, Shanghai (CN)

(72) Inventors: Xing Wang, Shanghai (CN); Dong Yang, Shanghai (CN); Hui Yang, Shanghai (CN)

(21) Appl. No.: 18/850,117

(22) PCT Filed: Mar. 28, 2023
(86) PCT No.: PCT/CN2023/084528

§ 371 Date: Sep. 24, 2024

(30) Foreign Application Priority Data
Mar. 28, 2022 (WO) PCT/CN2022/083477

(51) Int. Cl. A61K 48/00; A61K 9/00; A61P 25/28; C12N 9/22; C12N 15/11; C12N 15/86

(52) U.S. Cl. CPC A61K 48/0058; A61K 9/0085; A61K 48/0075; A61K 48/0083; A61P 25/28; C12N 9/22; C12N 15/111; C12N 15/86; C07K 2319/09; C12N 2310/20; C12N 2750/14143; C12N 2830/48; C12N 2830/50

(57) ABSTRACT

The disclosure provides a guide nucleic acid targeting human MECP2 mRNA, a system or composition or rAAV vector genome comprising the same, and a method using the same for treating human MECP2 mRNA associated disease and disorders.

### CROSS-REFERENCE TO RELATED APPLICATIONS

This application claims the benefit of and priority to PCT Patent Application No. PCT/CN2022/083477, filed on Mar. 28, 2022, entitled “CRISPR-CAS13 SYSTEM FOR TREATING MECP2-ASSOCIATED DISEASES”, which, including any sequence listing and drawings, is incorporated herein by reference in its entirety.

### REFERENCE TO AN ELECTRONIC SEQUENCE LISTING

The disclosure contains an electronic sequence listing (“xxx.xml” created on XX, by software “WIPO Sequence” according to WIPO Standard ST.26), which is incorporated herein by reference in its entirety. Wherever a sequence is an RNA sequence, the T in the sequence shall be deemed as U.

### BACKGROUND

MECP2 duplication syndrome (MDS) is a devastating childhood neurodevelopmental disorder, caused by the duplication of the gene encoding methyl-CpG binding protein 2 (MeCP2).

Citation or identification of any document in the disclosure is not an admission that such a document is available as prior art to the disclosure.

### SUMMARY

It is against the above background that the disclosure provides certain advantages over the prior art. Although the disclosure herein is not limited to specific advantages, in an aspect, the disclosure provides a guide nucleic acid comprising:
(1) a scaffold sequence capable of forming a complex with a nucleic acid programmable RNA nuclease (napRNAn), and
(2) a guide sequence capable of hybridizing to a target sequence of human MECP2 mRNA, thereby guiding the complex to the human MECP2 mRNA.

In another aspect, the disclosure provides a system or composition comprising:
(1) a guide nucleic acid, or a polynucleotide encoding the guide nucleic acid, comprising:
   (a) a scaffold sequence capable of forming a complex with a nucleic acid programmable RNA nuclease (napRNAn), and
   (b) a guide sequence capable of hybridizing to a target sequence of human MECP2 mRNA, thereby guiding the complex to the human MECP2 mRNA; and
(2) the napRNAn or a polynucleotide encoding the napRNAn.

In yet another aspect, the disclosure provides a recombinant adeno-associated virus (rAAV) vector genome comprising:
(a) a first polynucleotide sequence comprising a first sequence encoding a first guide nucleic acid comprising:
   (1) a scaffold sequence capable of forming a complex with a nucleic acid programmable RNA nuclease (napRNAn), and
   (2) a first guide sequence capable of hybridizing to a first target sequence of human MECP2 mRNA, thereby guiding the complex to the human MECP2 mRNA; and
(b) a second polynucleotide sequence comprising a sequence encoding the napRNAn,

wherein the rAAV vector genome is adapted to be encapsulated into a recombinant AAV particle.

In yet another aspect, the disclosure provides a recombinant AAV (rAAV) particle comprising the rAAV vector genome of the disclosure.

In yet another aspect, the disclosure provides a pharmaceutical composition comprising the system or composition of the disclosure or the rAAV particle of the disclosure and a pharmaceutically acceptable excipient.

In yet another aspect, the disclosure provides a method for preventing or treating a disease or disorder associated with human MECP2 mRNA in a subject in need thereof, comprising administering to the subject the system or composition of the disclosure, the rAAV particle of the disclosure, or the pharmaceutical composition of the disclosure, wherein the napRNAn modifies the human MECP2 mRNA, and wherein the modification of the human MECP2 mRNA treats the disease or disorder.

The details of one or more embodiments of the disclosure are set forth in the description below. Other features or advantages of the disclosure will be apparent from the following drawings and detailed description of several embodiments, and also from the appended claims. It is understood that any aspect or embodiment of the disclosure can be combined with any other aspect or embodiment of the disclosure to constitute another embodiment explicitly or implicitly disclosed herein unless otherwise indicated.

### Definitions

The disclosure will be described with respect to particular embodiments, but the disclosure is not limited thereto but only by the claims. Unless defined otherwise, all technical and scientific terms used herein have the same meaning as is commonly understood by one of ordinary skill in the art to which this disclosure belongs. Terms as set forth hereinafter are generally to be understood in their common sense unless indicated otherwise.

### Overview

Nucleic acid programmable RNA nuclease (napRNAn), for example, Cas13, is capable of cleaving a target RNA as guided by a guide nucleic acid (e.g., a guide RNA) comprising a guide sequence targeting the target RNA. In some embodiments, the target RNA is eukaryotic.

Without wishing to be bound by theory, in some embodiments, the guide nucleic acid comprises a scaffold sequence responsible for forming a complex with the napRNAn, and a guide sequence that is intentionally designed to be responsible for hybridizing to a target sequence of the target RNA, thereby guiding the complex comprising the napRNAn and the guide nucleic acid to the target RNA.

Referring to FIG. 10, an exemplary dsRNA is depicted to comprise a 5′ to 3′ single DNA strand and a 3′ to 5′ single DNA strand. According to conventional transcription process, an exemplary RNA transcript may be transcribed using the 3′ to 5′ single DNA strand as a synthesis template, and thus the 3′ to 5′ single DNA strand is referred to as a “template strand” or a “antisense strand”. The RNA transcript so transcribed has the same primary sequence as the 5′ to 3′ single DNA strand except for the replacement of T with U, and thus the 5′ to 3′ single DNA strand is referred to as a “coding strand” or a “sense strand”.

An exemplary guide nucleic acid is depicted to comprise a guide sequence and a scaffold sequence. The guide sequence is designed to hybridize to a part of the RNA transcript (target RNA), and so the guide sequence “targets” that part. And thus, that part of the target RNA based on which the guide sequence is designed and to which the guide sequence may hybridize is referred to as a “target sequence”. In some embodiments, the guide sequence is 100% (fully) reversely complementary to the target sequence. In some other embodiments, the guide sequence is reversely complementary to the target sequence and contains a mismatch with the target sequence (as exemplified in FIG. 11).

Generally, the double-strand sequence of a dsDNA may be represented with the sequence of its 5′ to 3′ single DNA strand conventionally written in 5′ to 3′ direction/orientation. Generally, a nucleic acid sequence (e.g., a DNA sequence, an RNA sequence) is written in 5′ to 3′ direction/orientation.

For example, for a dsDNA with a 5′ to 3′ single DNA strand of 5′-ATGC-3′ and a 3′ to 5′ single DNA strand of 3′-TACG-5′, the dsDNA may be simply represented as 5′-ATGC-3′. Normally, the RNA transcript (target RNA) transcribed from the dsDNA then has a sequence of 5′-AUGC-3′.

To hybridize to the target RNA, in one embodiment, the guide sequence of a guide nucleic acid is designed to have a sequence of 5′-GCAU-3′ that is fully reversely complementary to the target RNA. According to electric sequence listing standard ST.26 by WIPO, symbol “t” is used to denote both T in DNA and U in RNA (See “Table 1: List of nucleotides symbols”, the definition of symbol “t” is “thymine in DNA/uracil in RNA (t/u)”). Thus, in the sequence listing according to ST.26, such a guide sequence would be set forth in GCAT but marked as a RNA sequence.

### Term

As used herein, if a DNA sequence, for example, 5′-ATGC-3′ is transcribed to an RNA sequence, with each dT (deoxythymidine, or “T” for short) in the primary sequence replaced with a U (uridine) and other dA (deoxyadenosine, or “A” for short), dG (deoxyguanosine, or “G” for short), and dC (deoxycytidine, or “C” for short) replaced with A (adenosine), G (guanosine), and C (cytidine), respectively, for example, 5′-AUGC-3′, it is said in the disclosure that the DNA sequence “encodes” the RNA sequence.

As used herein, the term “activity” refers to a biological activity. In some embodiments, the activity includes enzymatic activity, e.g., catalytic ability of an effector. For example, the activity can include nuclease activity, e.g., RNA nuclease activity, RNA endonuclease activity.

As used herein, the term “complex” refers to a grouping of two or more molecules. In some embodiments, the complex comprises a polypeptide and a nucleic acid interacting with (e.g., binding to, coming into contact with, adhering to) one another. As used herein, the term “complex” can refer to a grouping of a guide nucleic acid and a polypeptide (e.g., a napRNAn, such as, a Cas13 polypeptide). As used herein, the term “complex” can refer to a grouping of a guide nucleic acid, a polypeptide, and a target sequence. As used herein, the term “complex” can refer to a grouping of a target RNA-targeting guide nucleic acid, a napRNAn, and optionally, a target RNA.

As used herein, the term “guide nucleic acid” refers to any nucleic acid that facilitates the targeting of a napRNAn (e.g., a Cas13 polypeptide) to a target sequence (e.g., a sequence of a target RNA). A guide nucleic acid may be designed to include a sequence that is complementary to a specific nucleic acid sequence (e.g., a sequence of a target RNA). A guide nucleic acid may comprise a scaffold sequence facilitating the guiding of a napRNAn to the target RNA. In some embodiments, the guide nucleic acid is a guide RNA.

As used herein, the terms “nucleic acid”, “polynucleotide”, and “nucleotide sequence” are used interchangeably to refer to a polymeric form of nucleotides of any length, including deoxyribonucleotides, ribonucleotides, combinations thereof, and analogs or modifications thereof.

As used in the context of CRISPR-Cas techniques (e.g., CRISPR-Cas13 techniques), the term “guide RNA” is used interchangeably with the term “CRISPR RNA (crRNA)”, “single guide RNA (sgRNA)”, or “RNA guide”, the term “guide sequence” is used interchangeably with the term “spacer sequence”, and the term “scaffold sequence” is used interchangeably with the term “direct repeat sequence”.

As described herein, the guide sequence is so designed to be capable of hybridizing to a target sequence. As used herein, the term “hybridize”, “hybridizing”, or “hybridization” refers to a reaction in which one or more polynucleotide sequences react to form a complex that is stabilized via hydrogen bonding between the bases of the polynucleotide sequences. The hydrogen bonding may occur by Watson Crick base pairing, Hoogstein binding, or in any other sequence specific manner. A polynucleotide sequence capable of hybridizing to a given polynucleotide sequence is referred to as the “complement” of the given polynucleotide sequence. As used herein, the hybridization of a guide sequence and a target sequence is so stabilized to permit an effector polypeptide (e.g., a napRNAn) that is complexed with a nucleic acid comprising the guide sequence or a function domain associated (e.g., fused) with the effector polypeptide to act (e.g., cleave, deaminize) on the target sequence or its complement (e.g., a sequence of a target RNA or its complement).

For the purpose of hybridization, in some embodiments, the guide sequence is complementary or reversely complementary to a target sequence. As used herein, the term “complementary” refers to the ability of nucleobases of a first polynucleotide sequence, such as a guide sequence, to base pair with nucleobases of a second polynucleotide sequence, such as a target sequence, by traditional Watson-Crick base-pairing. Two complementary polynucleotide sequences are able to non-covalently bind under appropriate temperature and solution ionic strength conditions. In some embodiments, a first polynucleotide sequence (e.g., a guide sequence) comprises 100% (fully) complementarity to a second nucleic acid (e.g., a target sequence). In some embodiments, a first polynucleotide sequence (e.g., a guide sequence) is complementary to a second polynucleotide sequence (e.g., a target sequence) if the first polynucleotide sequence comprises at least about 80%, 81%, 82%, 83%, 84%, 85%, 86%, 87%, 88%, 89%, 90%, 91%, 92%, 93%, 94%, 95%, 96%, 97%, 98%, or 99% complementarity to the second nucleic acid. As used herein, the term “substantially complementary” refers to a polynucleotide sequence (e.g., a guide sequence) that has a certain level of complementarity to a second polynucleotide sequence (e.g., a target sequence). In some embodiments, the level of complementarity is such that the first polynucleotide sequence (e.g., a guide sequence) can hybridize to the second polynucleotide sequence (e.g., a target sequence) with sufficient affinity to permit an effector polypeptide (e.g., a napRNAn) that is complexed with the first polynucleotide sequence or a nucleic acid comprising the first polynucleotide sequence or a function domain associated (e.g., fused) with the effector polypeptide to act (e.g., cleave, deaminize) on the target sequence or its complement (e.g., a sequence of a target RNA or its complement). In some embodiments, a guide sequence that is substantially complementary to a target sequence has less than 100% complementarity to the target sequence. In some embodiments, a guide sequence that is substantially complementary to a target sequence has at least about 80%, 81%, 82%, 83%, 84%, 85%, 86%, 87%, 88%, 89%, 90%, 91%, 92%, 93%, 94%, 95%, 96%, 97%, 98%, or 99% complementarity to the target sequence.

As used herein, the term “sequence identity” is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences.

Sequence homologies may be generated by any of a number of computer programs known in the art, for example BLAST or FASTA, etc. A suitable computer program for carrying out such an alignment is the GCG Wisconsin Bestfit package (University of Wisconsin, U.S.A; Devereux et al., 1984, Nucleic Acids Research 12:387). Examples of other software than may perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). Percentage (%) sequence homology may be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues. Although this is a very simple and consistent method, it fails to take into consideration that, for example, in an otherwise identical pair of sequences, one insertion or deletion may cause the following amino acid residues to be put out of alignment, thus potentially resulting in a large reduction in % homology when a global alignment is performed. Consequently, most sequence comparison methods are designed to produce optimal alignments that take into consideration possible insertions and deletions without unduly penalizing the overall homology or identity score. This is achieved by inserting “gaps” in the sequence alignment to try to maximize local homology or identity. However, these more complex methods assign “gap penalties” to each gap that occurs in the alignment so that, for the same number of identical amino acids, a sequence alignment with as few gaps as possible-reflecting higher relatedness between the two compared sequences—may achieve a higher score than one with many gaps. “Affinity gap costs” are typically used that charge a relatively high cost for the existence of a gap and a smaller penalty for each subsequent residue in the gap. This is the most commonly used gap scoring system. High gap penalties may, of course, produce optimized alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension. Calculation of maximum % homology therefore first requires the production of an optimal alignment, taking into consideration gap penalties. A new tool, called BLAST 2 Sequences is also available for comparing protein and nucleotide sequences (see FEMS Microbiol Lett. 1999 174(2): 247-50; FEMS Microbiol Lett. 1999 177(1): 187-8 and the website of the National Center for Biotechnology information at the website of the National Institutes for Health). Although the final % homology may be measured in terms of identity, the alignment process itself is typically not based on an all-or-nothing pair comparison. Instead, a scaled similarity score matrix is generally used that assigns scores to each pair-wise comparison based on chemical similarity or evolutionary distance. An example of such a matrix commonly used is the BLOSUM62 matrix the default matrix for the BLAST suite of programs. GCG Wisconsin programs generally use either the public default values or a custom symbol comparison table, if supplied (see user manual for further details). For some applications, it is preferred to use the public default values for the GCG package, or in the case of other software, the default matrix, such as BLOSUM62. Alternatively, percentage homologies may be calculated using the multiple alignment feature in DNASIS™ (Hitachi Software), based on an algorithm, analogous to CLUSTAL (Higgins D G & Sharp P M (1988), Gene 73(1), 237-244). Once the software has produced an optimal alignment, it is possible to calculate % homology, preferably % sequence identity. The software typically does this as part of the sequence comparison and generates a numerical result. The sequences may also have deletions, insertions or substitutions of amino acid residues which produce a silent change and result in a functionally equivalent substance. Deliberate amino acid substitutions may be made on the basis of similarity in amino acid properties (such as polarity, charge, solubility, hydrophobicity, hydrophilicity, and/or the amphipathic nature of the residues) and it is therefore useful to group amino acids together in functional groups. Amino acids may be grouped together based on the properties of their side chains alone. However, it is more useful to include mutation data as well. The sets of amino acids thus derived are likely to be conserved for structural reasons. These sets may be described in the form of a Venn diagram (Livingstone C. D. and Barton G. J. (1993) “Protein sequence alignments: a strategy for the hierarchical analysis of residue conservation” Comput. Appl. Biosci. 9: 745-756) (Taylor W. R. (1986) “The classification of amino acid conservation” J. Theor Biol. 119; 205-218). Conservative substitutions may be made, for example according to the table below which describes a generally accepted Venn diagram grouping of amino acids.

As used herein, the terms “polypeptide” and “peptide” are used interchangeably herein to refer to polymers of amino acids of any length. The polymer may be linear or branched, it may comprise modified amino acids, and it may be interrupted by non-amino acids. A protein may have one or more polypeptides. The terms also encompass an amino acid polymer that has been modified; for example, by disulfide bond formation, glycosylation, lipidation, acetylation, phosphorylation, or any other manipulation, such as conjugation with a labeling component.

As used herein, a “variant” is interpreted to mean a polynucleotide or polypeptide that differs from a reference polynucleotide or polypeptide, respectively, but retains essential properties. A typical variant of a polynucleotide differs in nucleic acid sequence from another, reference polynucleotide. Changes in the nucleic acid sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

As used herein, the terms “upstream” and “downstream” refer to relative positions within a single nucleotide (e.g., DNA) sequence in a nucleic acid. Generally, a first sequence “upstream” of a second sequence means that the first sequence is 5′ to the second sequence, and a first sequence “downstream” of a second sequence means that the first sequence is 3′ to the second sequence.

As used herein, the term “wild type” has the meaning commonly understood by those skilled in the art to mean a typical form of an organism, a strain, a gene, or a feature that distinguishes it from a mutant or variant when it exists in nature. It can be isolated from sources in nature and not intentionally modified.

As used herein, the terms “non-naturally occurring” and “engineered” are used interchangeably and refer to artificial participation. When these terms are used to describe a nucleic acid or a polypeptide, it is meant that the nucleic acid or polypeptide is at least substantially freed from at least one other component of its association in nature or as found in nature.

As used herein, the “cell” is understood to refer not only to a particular individual cell, but to the progeny or potential progeny of the cell. Because certain modifications may occur in succeeding generations due to either mutation or environmental influences, such progeny may not, in fact, be identical to the parent cell, but are still included within the scope of the term.

As used herein, the term “in vivo” refers to inside the body of an organism, and the terms “ex vivo” or “in vitro” means outside the body of an organism.

As used herein, the term “treat”, “treatment”, or “treating” is an approach for obtaining beneficial or desired results including clinical results. For purposes of the disclosure, the beneficial or desired clinical results include, but are not limited to, one or more of the following: alleviating one or more symptoms resulting from a disease, diminishing the extent of a disease, stabilizing a disease (e.g., preventing or delaying the worsening of a disease), preventing or delaying the spread (e.g., metastasis) of a disease, preventing or delaying the recurrence of a disease, reducing recurrence rate of a disease, delay or slowing the progression of a disease, ameliorating a disease state, providing a remission (partial or total) of a disease, decreasing the dose of one or more other medications required to treat a disease, delaying the progression of a disease, increasing the quality of life, and/or prolonging survival. Also encompassed by “treatment” is a reduction of pathological consequence of a disease (such as cancer). The methods of the disclosure contemplate any one or more of these aspects of treatment.

As used herein, the term “disease” includes the terms “disorder” and “condition” and is not limited to those have been specifically medically defined.

As used herein, reference to “not” a value or parameter generally means and describes “other than” a value or parameter. For example, the method is not used to treat cancer of type X means the method may be used to treat cancer of types other than X.

As used herein, the singular forms “a”, “an”, and “the” include plural referents unless the context clearly dictates otherwise.

As used herein, the term “and/or” in a phrase such as “A and/or B” is intended to include both A and B; A or B; A (alone); and B (alone). Likewise, the term “and/or” in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, when the term “about” is ahead of a serious of numbers (for example, about 1, 2, 3), it is understood that each of the serious of numbers is modified by the term “about” (that is, about 1, about 2, about 3). The term “about X-Y” used herein has the same meaning as “about X to about Y.” It is understood that embodiments of the disclosure described herein include “consisting” and/or “consisting essentially of” embodiments.

It is further noted that the claims may be drafted to exclude any optional element. As such, this statement is intended to serve as antecedent basis for use of such exclusive terminology as “solely”, “only”, and the like in connection with the recitation of claim elements, or use of a “negative” limitation.

### BRIEF DESCRIPTION OF THE DRAWINGS

An understanding of the features and advantages of the disclosure will be obtained by reference to the following detailed description that sets forth illustrative embodiments, in which the principles of the disclosure may be utilized, and the accompanying drawings of which:

FIG. 1A-1C are schematic (not to scale) illustrations of constructs expressing hfCas13f v2 and sgRNA[MECP2] containing one or two Spacers or sgRNA [NT](as a negative control) with mCherry as a reporter.

FIG. 2A-2C shows graphs of in vitro knockdown of MECP2 mRNA level by hfCas13f v2-sgRNA[MECP2] compared to the negative control. MECP2 mRNA levels were detected by RT-qPCR. N=3-4/group. P<0.05; **, P<0.01.

FIG. 3A is a schematic (not to scale) illustration of a AAV.PHP.eB construct expressing hfCas13f v2 and sgRNA[MECP2] containing two Spacers 9 & 3; FIG. 3B shows an illustration of unilateral stereotactic injection of AAV particles into the hippocampus of mouse; FIG. 3C-3E show graphs of in vivo knockdown of MECP2 mRNA and protein levels by stereotactic injection of AAV.PHP.eB particles delivering hfCas13f v2-sgRNA[MECP2]9 & 3 into Hips DG regions of C57BL/6 mice, as compared to untreated side. MECP2 mRNA and protein levels were detected by RT-qPCR and IF, respectively. N=4/group. **, P<0.01; ***, P<0.001.

FIG. 4A is a schematic (not to scale) illustration of a AAV.PHP.eB construct expressing hfCas13f v2.5 and sgRNA[MECP2] containing two Spacers 9 & 3; FIG. 4B shows an illustration of unilateral stereotactic injection of AAV particles into the hippocampus of C57BL/6 mouse; FIG. 4C-4E show graphs of in vivo knockdown of MECP2 mRNA and protein levels by stereotactic injection of AAV.PHP.eB particles delivering hfCas13f v2.5-sgRNA[MECP2]9 & 3 into Hip DG regions of C57BL/6 mice, as compared to the uninjected side of the same Hip as a negative control. MECP2 mRNA and protein levels were detected by RT-qPCR and IF, respectively. N=2-3/group. **, P<0.01.

FIG. 5A-5C show graphs of the infection efficiency of AAV.PHP.eB particles delivering hfCas13f v2.5 system in MDS mice with ICV administration. hfCas13f v2.5 tagged with HA was detected by RT-qPCR (FIG. 5B), Western blot (FIG. 5C), and IF (FIG. 5A); FIG. 5D is a schematic (not to scale) illustration of a AAV.PHP.eB construct expressing hfCas13f v2.5 and sgRNA[MECP2] containing two Spacers 9 & 3 or sgRNA[NT] (as a negative control).

FIG. 6A-6C show graphs of in vivo knockdown of MECP2 by ICV administration of AAV.PHP.eB particles delivering hfCas13f v2.5 system into the Lateral Ventricles to infect the whole brain of MDS mice, as compared to the negative control with ICV injection of control AAV.PHP.eB particles. MECP2 protein levels were detected by Western blot and IF. N=2-3/group in the Western Blot experiment.

FIG. 7A-7B show that the abnormal motor and anxiety-like behavior of MDS mice was partly rescued at Week 4 or Week 8 after ICV administration. N=5/group.

FIG. 8A-8D show that the abnormal motor and social behaviors of MDS mice were reversed at Week 10 after ICV administration. N=5/group. *, P<0.05; **, P<0.01; ****, P<0.0001.

FIG. 9 shows that there was no observed toxicity for the AAV.PHP.eB particles at Week 7 after AAV ICV administration.

FIG. 10 is a schematic showing an exemplary dsDNA, an exemplary RNA transcript transcribed from the dsDNA, an exemplary guide nucleic acid, and an exemplary napRNAn, wherein the guide sequence is reversely complementary to the target sequence.

FIG. 11 is a schematic showing an exemplary dsDNA, an exemplary RNA transcript transcribed from the dsDNA, an exemplary guide nucleic acid, and an exemplary napRNAn, wherein the guide sequence is reversely complementary to the target sequence and contains a mismatch with the target sequence.

FIG. 12-14 show the comparison of hfCas13f.1 v2 and v3.

FIG. 15 shows the whole transcriptome sequencing results of hfCas13f.1 v3.

The figures herein are for illustrative purposes only and are not necessarily drawn to scale.

### DETAILED DESCRIPTION

### Overview

The disclosure provides tools and methods for treatment of MECP2-associated diseases, such as, MECP2 Duplication Syndrome (MDS).

Specifically, the disclosure provides tools for downregulating the expression of MECP2 gene by cleaving/degrading MECP2 mRNA. Such tools can be delivered, for example, by AAV vectors and intracerebroventricular injection to subjects in need. Exemplary constructs of the disclosure have demonstrated efficacy to knockdown MECP2 mRNA level, especially in vivo, and improve disease phenotypes of mouse models, thus opening the door for gene therapy to treat MECP2-assocaited diseases, such as, MDS.

### Guide Nucleic Acid

In an aspect, the disclosure provides a guide nucleic acid comprising:
(1) a scaffold sequence capable of forming a complex with a nucleic acid programmable RNA nuclease (napRNAn), and
(2) a guide sequence capable of hybridizing to a target sequence of human MECP2 mRNA, thereby guiding the complex to the human MECP2 mRNA.

### System or Composition

In another aspect, the disclosure provides a system or composition comprising:
(1) a guide nucleic acid, or a polynucleotide encoding the guide nucleic acid, comprising:
(a) a scaffold sequence capable of forming a complex with a nucleic acid programmable RNA nuclease (napRNAn), and
(b) a guide sequence capable of hybridizing to a target sequence of human MECP2 mRNA, thereby guiding the complex to the human MECP2 mRNA; and
(2) the napRNAn or a polynucleotide encoding the napRNAn.

In some embodiments, the system is a complex comprising the napRNAn complexed with the guide nucleic acid. In some embodiments, the complex further comprises the human MECP2 mRNA hybridized with the target sequence.

rAAV Vector Genome

In yet another aspect, the disclosure provides a recombinant adeno-associated virus (rAAV) vector genome comprising:
(a) a first polynucleotide sequence comprising a first sequence encoding a first guide nucleic acid comprising:
(1) a scaffold sequence capable of forming a complex with a nucleic acid programmable RNA nuclease (napRNAn), and
(2) a first guide sequence capable of hybridizing to a first target sequence of human MECP2 mRNA, thereby guiding the complex to the human MECP2 mRNA; and
(b) a second polynucleotide sequence comprising a sequence encoding the napRNAn, wherein the rAAV vector genome is adapted to be encapsulated into a recombinant AAV particle.

### Multiple Guide Nucleic Acid

The system, composition, or rAAV vector genome of the disclosure may comprise or encode one guide nucleic acid or encode more than one nucleic acid, e.g., for the purpose of improving cleavage efficiency against human MECP2 mRNA.

In some embodiments, the first polynucleotide sequence further comprises a second sequence encoding a second guide nucleic acid comprising:
(1) a or the scaffold sequence capable of forming a complex with a napRNAn, and
(2) a second guide sequence capable of hybridizing to a second target sequence of human MECP2 mRNA, thereby guiding the complex to the human MECP2 mRNA.

In some embodiments, the first polynucleotide sequence further comprises a third sequence encoding a third guide nucleic acid comprising:
(1) a or the scaffold sequence capable of forming a complex with a napRNAn, and
(2) a third guide sequence capable of hybridizing to a third target sequence of human MECP2 mRNA, thereby guiding the complex to the human MECP2 mRNA.

In some embodiments, the first polynucleotide sequence further comprises a fourth sequence encoding a fourth guide nucleic acid comprising:
(1) a or the scaffold sequence capable of forming a complex with a napRNAn, and
(2) a fourth guide sequence capable of hybridizing to a fourth target sequence of human MECP2 mRNA, thereby guiding the complex to the human MECP2 mRNA.

In some embodiments, the first polynucleotide sequence further comprises a firth, a sixth, a seventh guide nucleic acid, and so on.

In some embodiments that the same napRNAn is used, the scaffold sequences of the more than one guide nucleic acids may be the same or slightly different (e.g., different by no more than 5, 4, 3, 2, or 1 nucleotide) to be compatible to the same napRNAn. In some embodiments that different napRNAn are used, the scaffold sequences of the more than one guide nucleic acids may be different to be compatible to the different napRNAn.

### Nature of the Guide Nucleic Acid of the Disclosure

In some embodiments, the guide nucleic acid (e.g., the guide nucleic acid, the first guide nucleic acid, the second guide nucleic acid, the third guide nucleic acid, the fourth guide nucleic acid, and so on; same as elsewhere) is an RNA. In some embodiments, the guide nucleic acid comprises a modification. In some embodiments, the guide nucleic acid is an unmodified RNA or modified RNA. In some embodiments, the guide nucleic acid is a modified RNA containing a modified ribonucleotide. In some embodiments, the guide nucleic acid is a modified RNA containing a deoxyribonucleotide. In some embodiments, the guide nucleic acid is a modified RNA containing a modified deoxyribonucleotide. In some embodiments, the guide nucleic acid comprises a modified or unmodified deoxyribonucleotide and a modified or unmodified ribonucleotide.

### Mechanism of the Disclosure

Methyl-CpG binding protein 2 (MeCP2), an epigenetic regulator that is crucial for normal brain development and function, is encoded by the dosage-sensitive MECP2 gene involved in two devastating childhood neurodevelopmental disorders. Loss-of-function mutations in the MECP2 gene cause the Rett syndrome (RTT), whereas duplication on chromosome Xq28 spanning the MECP2 gene causes MECP2 duplication syndrome (MDS).

MDS is one of the most common subtelomeric genomic rearrangements in males, accounting for about 1% of X-linked cases of intellectual disability Inherited in an X-linked manner, MDS is believed to be completely penetrant in males. The cardinal features of MDS include infantile hypotonia, intellectual disability, anxiety, motor dysfunction, epilepsy (about 50% of affected individuals) and recurrent respiratory tract infections (about 75%). Approximately half of affected individuals succumb before the age of 25, and usually die of respiratory infections. However, this statistic underestimates the proportion of early deaths because most of the patients are young children. To date, more than 600 cases worldwide have been reported and the clinical pathology are consistent in all reports.

MeCP2 regulates gene expression through binding methylated DNA. Latest findings suggest that the transcription factor 20 (TCF20) complex interacts with MeCP2 at the chromatin interface and modifies MeCP2-induced synaptic and behavioral deficits. Studies have shown that hundreds of genes may be regulated by MeCP2 in a cell-specific manner. While MeCP2 is ubiquitously expressed in various human tissues, it is abundantly expressed in the brain, and among which neurons have the highest level of MeCP2. Overexpression of MeCP2 could have detrimental effects on brain development as shown in mouse models and MDS patient.

It is recommended that lifelong multidisciplinary approach should be conducted in MDS patient. The existing therapies are usually symptomatic and the efficacy is limited. For instance, 82% of MDS-related epilepsy is treatment-refractory and consistent with epileptic encephalopathy. Since the clinical symptoms tend to get worse with age, burden on MDS parents and their caregivers is significant, expressing top concerns as related to lack of effective communication, abnormal walking/balance issues and seizures. Therefore, there remains high unmet clinical needs for the treatment of MDS.

As described above, the guide sequence of the guide nucleic acid is designed to be capable of hybridizing to the human MECP2 mRNA, thereby guiding the complex comprising the guide nucleic acid and the napRNAn to the human MECP2 mRNA. In some embodiments, said guiding the complex to the human MECP2 mRNA enables the napRNAn to specifically cleave the human MECP2 mRNA. In some embodiments, the specific cleavage of the human MECP2 mRNA leads to degradation of the human MECP2 mRNA. In the case that the level of human MECP2 mRNA is properly reduced, it is reasonably expected that the syndromes of a subject caused by improper level of human MECP2 mRNA can be alleviated.

### Design of the Target Sequence of the Disclosure, Target Site

For the purpose of the disclosure, in some embodiments, the target sequence (e.g., the target sequence, the first target sequence, the second target sequence, the third target sequence, the fourth target sequence, and so on; same as elsewhere) is located such that the human MECP2 mRNA is specifically cleaved by the napRNAn.

To facilitate the evaluation of the designed target sequence in mouse models, in some embodiments, the target sequence is located such that a mouse MECP2 mRNA is specifically cleaved by the napRNAn. In some embodiments, the target sequence is located such that both the human MECP2 mRNA and a mouse MECP2 mRNA is specifically cleaved by the napRNAn. That is, the target sequence is selected to be cross-reactive to both human and mouse.

As used herein, the term “MECP2 mRNA” includes MECP2 mRNA, and any variants, derivatives, or ancestors thereof, including MECP2 pre-mRNA, and any transcripts or isoforms produced from MECP2 gene or MECP2 pre-mRNA by, e.g., alternative promoter usage, alternative splicing, alternative initiation, and any naturally occurring variants thereof or processed products therefrom. In some embodiments, the human MECP2 mRNA comprises a sequence of SEQ ID NO: 60.

### Structure of the Guide Nucleic Acid of the Disclosure

In some embodiments, the guide nucleic acid comprises the scaffold sequence 5′ or 3′ to the guide sequence (e.g., the guide sequence, the first guide sequence, the second guide sequence, the third guide sequence, the fourth guide sequence, and so on; same as elsewhere). In some embodiments, the guide nucleic acid comprises the scaffold sequence 3′ to the guide sequence. In some embodiments, the scaffold sequence is fused to the guide sequence without a linker.

In some embodiments, the guide nucleic acid comprises, from 5′ to 3′, one guide sequence and one scaffold sequence.

In some embodiments, the guide nucleic acid comprises, from 5′ to 3′, one scaffold sequence, one guide sequence, and one scaffold sequence, wherein the scaffold sequences are the same or different.

In some embodiments, the guide nucleic acid comprises, from 5′ to 3′, one scaffold sequence, one guide sequence, one scaffold sequence, and one guide sequence, wherein the scaffold sequences are the same or different, and wherein the guide sequences are the same or different.

In some embodiments, the guide nucleic acid comprises, from 5′ to 3′, one scaffold sequence, one guide sequence, one scaffold sequence, one guide sequence, and one scaffold sequence, wherein the scaffold sequences are the same or different, and wherein the guide sequences are the same or different.

In some embodiments, the guide nucleic acid comprises, from 5′ to 3′, one scaffold sequence, one guide sequence, one scaffold sequence, one guide sequence, one scaffold sequence, and one guide sequence, wherein the scaffold sequences are the same or different, and wherein the guide sequences are the same or different.

### Length of the Target and Guide Sequences of the Disclosure

In some embodiments, the target sequence is at least about 14 nucleotides in length, e.g., about 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more nucleotides in length, or in a length of a numerical range between any of two preceding values, e.g., in a length of from about 16 to about 50 nucleotides. In some embodiments, the target sequence is about 30 nucleotides in length.

In some embodiments, the target sequence comprises, consists essentially of, or consists of at least about 14 contiguous nucleotides of the human MECP2 mRNA (e.g., about 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides of the human MECP2 mRNA, or in a numerical range between any of two preceding values, e.g., from about 14 to about 50 contiguous nucleotides of the human MECP2 m-RNA). In some embodiments, the target sequence comprises, consists essentially of, or consists of about 30 contiguous nucleotides of the human MECP2 mRNA.

In some embodiments, the guide sequence is at least about 14 nucleotides in length, e.g., about 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more nucleotides in length, or in a length of a numerical range between any of two preceding values, e.g., in a length of from about 16 to about 50 nucleotides. In some embodiments, the guide sequence is about 30 nucleotides in length.

In some embodiments, the guide sequence is at least about 50%, 55%, 60%, 65%, 70%, 75%, 80%, 85%, 90%, 95%, or 100% (fully), optionally about 100% (fully), complementary to the target sequence; or wherein the guide sequence comprises no mismatch with the target sequence in the first 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides at the 5′ end of the guide sequence. It is generally believed that at least 2 mismatches between the guide sequence and the target sequence can be tolerated for the napRNAn (e.g., Cas13) of the disclosure without significantly decreasing cleavage activity. In some embodiments, the guide sequence is 100% (fully) complementary to the target sequence.

### Specific Embodiments of the Target and Guide Sequences of the Disclosure

In some embodiments, the target sequence comprises, consists essentially of, or consists of (1) a sequence of any one of SEQ ID NOs: 1-10 or a 5′ or 3′ end truncation thereof with 1, 2, 3, 4, 5, or 6, nucleotides truncated at the 5′ or 3′ end; or (2) a sequence having a sequence identity of at least about 70%, 75%, 80%, 85%, 90%, 95%, or 100% to any one of SEQ ID NOs: 1-10 or a 5′ or 3′ end truncation thereof with 1, 2, 3, 4, 5, or 6 nucleotides truncated at the 5′ or 3′ end; or (3) a sequence having at most 1, 2, 3, 4, 5, 6, 7, 8, 9, or 10 nucleotide differences, whether consecutive or not, compared to any one of SEQ ID NOs: 1-10.

In some embodiments, the target sequence comprises the sequence of SEQ ID NO: 29 or 35.

In some embodiments, the guide sequence comprises, consists essentially of, or consists of (1) a sequence of any one of SEQ ID NOs: 17-26 or a 5′ or 3′ end truncation thereof with 1, 2, 3, 4, 5, or 6, nucleotides truncated at the 5′ or 3′ end; or (2) a sequence having a sequence identity of at least about 70%, 75%, 80%, 85%, 90%, 95%, or 100% to any one of SEQ ID NOs: 17-26 or a 5′ or 3′ end truncation thereof with 1, 2, 3, 4, 5, or 6 nucleotides truncated at the 5′ or 3′ end; or (3) a sequence having at most 1, 2, 3, 4, 5, 6, 7, 8, 9, or 10 nucleotide differences, whether consecutive or not, compared to any one of SEQ ID NOs: 17-26.

In some embodiments, the guide sequence comprises the sequence of SEQ ID NO: 19 or 25.

### Specific Embodiments of the Scaffold Sequence of the Disclosure

For the purpose of the disclosure, the scaffold sequence is compatible with the napRNAn of the disclosure and is capable of complexing with the napRNAn. The scaffold sequence may be a naturally occurring scaffold sequence identified along with the napRNAn, or a variant thereof maintaining the ability to complex with the napRNAn. Generally, the ability to complex with the napRNAn is maintained as long as the secondary structure of the variant is substantially identical to the secondary structure of the naturally occurring scaffold sequence. A nucleotide deletion, insertion, or substitution in the primary sequence of the scaffold sequence may not necessarily change the secondary structure of the scaffold sequence (e.g., the relative locations and/or sizes of the stems, bulges, and loops of the scaffold sequence do not significantly deviate from that of the original stems, bulges, and loops). For example, the nucleotide deletion, insertion, or substitution may be in a bulge or loop region of the scaffold sequence so that the overall symmetry of the bulge and hence the secondary structure remains largely the same. The nucleotide deletion, insertion, or substitution may also be in the stems of the scaffold sequence so that the lengths of the stems do not significantly deviate from that of the original stems (e.g., adding or deleting one base pair in each of two stems correspond to 4 total base changes).

In some embodiments, the scaffold sequence has substantially the same secondary structure as the secondary structure of the sequence of SEQ ID NO: 6.

In some embodiments, the scaffold sequence comprises, consists essentially of, or consists of a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 6; or a sequence having at most 1, 2, 3, 4, 5, 6, 7, 8, 9, or 10 nucleotide differences, whether consecutive or not, compared to the sequence of SEQ ID NO: 6.

In some embodiments, the scaffold sequence comprises the sequence of SEQ ID NO: 6.

### Specific Embodiments of the Guide Nucleic Acid of the Disclosure

In some embodiments, the guide nucleic acid comprises, consists essentially of, or consists of a sequence having a sequence identity of at least about 80% (e.g., at least about 80%, 85%, 90%, 91%, 92%, 93%, 94%, 95%, 96%, 97%, 98%, 99%, or 100%) to the sequence of any of SEQ ID NOs: 7-16 and 37-40; or a sequence having at most 1, 2, 3, 4, 5, 6, 7, 8, 9, or 10 nucleotide differences, whether consecutive or not, compared to the sequence of any of SEQ ID NOs: 7-16 and 37-40.

In some embodiments, the guide nucleic acid comprises the sequence of SEQ ID NO: 37.

Regulation of the First Polynucleotide Sequence of the rAAV Vector Genome of the Disclosure

In some embodiments, the first polynucleotide sequence comprises a promoter operably linked to the sequence encoding the guide nucleic acid.

In some embodiments, the promoter is a ubiquitous, tissue-specific, cell-type specific, constitutive, or inducible promoter.

Suitable promoters are known in the art and include, for example, a Cbh promoter, a Cba promoter, a pol I promoter, a pol II promoter, a pol III promoter, a T7 promoter, a U6 promoter, a H1 promoter, a retroviral Rous sarcoma virus LTR promoter, a cytomegalovirus (CMV) promoter, a SV40 promoter, a dihydrofolate reductase promoter, a β-actin promoter, an elongation factor 1a short (EFS) promoter, a β glucuronidase (GUSB) promoter, a cytomegalovirus (CMV) immediate-early (Ie) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, and a myelin basic protein (MBP) promoter. In some embodiments, the promoter is a U6 promoter.

In some embodiments, the promoter comprises, consists essentially of, or consists of a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 49; or a sequence having at most 1, 2, 3, 4, 5, 6, 7, 8, 9, or 10 nucleotide differences, whether consecutive or not, compared to the sequence of SEQ ID NO: 49.

In some embodiments, the first polynucleotide sequence comprises, from 5′ to 3′, a promoter of SEQ ID NO: 49, and a sequence encoding the guide nucleic acid of any one of SEQ ID NOs: 7-16 and 37-40.

napRNAn of the Disclosure

For the purpose of the disclosure, the napRNAn is capable of forming a complex with the guide nucleic acid of the disclosure by complexing with the scaffold sequence of the guide nucleic acid and is thereby guided to the human MECP2 mRNA by the hybridization of the guide sequence of the guide nucleic acid to the target sequence of the human MECP2 mRNA. When the napRNAn is guided to the human MECP2 mRNA, the nuclease activity of the napRNAn functions to cleave the human MECP2 mRNA, leading to degradation of the human MECP2 mRNA. Generally, any such napRNAn can be used with the guide nucleic acid of the disclosure, and when a napRNAn is selected, the scaffold sequence compatible to the napRNAn for complexing with the napRNAn can also be selected accordingly. The scaffold sequence is generally conserved.

In some embodiments, the napRNAn is a Class 2, Type VI CRISPR-associated protein (Cas13). Cas13, a class 2 type VI RNA endonuclease, can bind and cleave single-stranded RNA guided by an engineered CRISPR RNA (crRNA). Gene therapies based on RNA-targeting nucleases are currently under exploration as a safer alternative to DNA editing Cas endonucleases, since they are accompanied by a lower risk of introducing permanent genomic alterations.

In some embodiments, the Cas13 is a Cas13a (C2c2), Cas13b (such as, Cas13b1, Cas13b2), Cas13c, Cas13d, Cas13e, or Cas13f polypeptide. To date, Cas13f.1 (Cas13Y.1, Cas13Y) is one of the smallest known crRNA-guided RNA endonucleases, with only 790 amino acids, and several high-fidelity variants of this Cas, collectively hfCas13f.1 (hfCas13Y.1), has been shown to exhibit high on-target activity with markedly lower collateral activity.

In some embodiments, the napRNAn comprises, consists essentially of, or consists of a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 1, 2, 4, or 61 or a N-terminal truncation thereof without the first N-terminal Methionine, and retaining at least 80% (e.g., at least about 80%, 85%, 90%, 91%, 92%, 93%, 94%, 95%, 96%, 97%, 98%, 99%, or 100%) of the guide sequence-specific RNA cleavage activity of the sequence of SEQ ID NO: 1, 2, 4, or 61.

In some embodiments, the napRNAn comprises the sequence of SEQ ID NO: 61.

In some embodiments, the sequence encoding the napRNAn comprises, consists essentially of, or consists of a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 3, 5, or 62 or a 5′ end truncation thereof without the first 5′ ATG codon, and wherein the napRNAn encoded by the sequence retains at least 80% (e.g., at least about 80%, 85%, 90%, 91%, 92%, 93%, 94%, 95%, 96%, 97%, 98%, 99%, or 100%) of the guide sequence-specific RNA cleavage activity of the sequence of SEQ ID NO: 3, 5, or 62.

In some embodiments, the sequence encoding the napRNAn comprises the sequence of SEQ ID NO: 62.

### Regulation of the Second Polynucleotide Sequence of the Disclosure

In some embodiments, the second polynucleotide sequence comprises a promoter operably linked to the sequence encoding the napRNAn.

In some embodiments, the promoter is a ubiquitous, tissue-specific, cell-type specific, constitutive, or inducible promoter.

Suitable promoters are known in the art and include, for example, a Cbh promoter, a Cba promoter, a pol I promoter, a pol II promoter, a pol III promoter, a T7 promoter, a U6 promoter, a H1 promoter, a retroviral Rous sarcoma virus LTR promoter, a cytomegalovirus (CMV) promoter, a SV40 promoter, a dihydrofolate reductase promoter, a β-actin promoter, an elongation factor 1a short (EFS) promoter, a β glucuronidase (GUSB) promoter, a cytomegalovirus (CMV) immediate-early (Ie) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, a myelin basic protein (MBP) promoter, a HTT promoter, a GRK1 promoter, a CRX promoter, a NRL promoter, a MECP2 promoter, a mMECP2 promoter, a hMECP2 promoter, and a RCVRN promoter.

In some embodiments, the promoter is a nerve cell (e.g., neuron) specific promoter, e.g., MECP2 promoter.

In some embodiments, the promoter is a Cbh promoter, a CMV promoter, a mMECP2 promoter, or a hMECP2 promoter.

In some embodiments, the second polynucleotide sequence comprises a Kozak sequence 5′ to the sequence encoding the napRNAn.

In some embodiments, the second polynucleotide sequence comprises a sequence encoding a nuclear localization signal (NLS) 5′ and/or 3′ to the sequence encoding the napRNAn.

In some embodiments, the second polynucleotide sequence comprises a sequence encoding a nuclear export signal (NES) 5′ and/or 3′ to the sequence encoding the napRNAn.

In some embodiments, the second polynucleotide sequence comprises a sequence encoding a first NLS 5′ to the sequence encoding the napRNAn and a second sequence encoding a second NLS 3′ to the sequence encoding the napRNAn.

In some embodiments, the NLS, the first NLS, and/or the second NLS is a SV40 NLS, a bpSV40 NLS, or a Nucleoplasmin NLS (npNLS).

In some embodiments, the second polynucleotide sequence comprises a WPRE sequence downstream of the sequence encoding the napRNAn.

In some embodiments, the WPRE sequence is selected from the group consisting of Woodchuck Hepatitis Virus (WHP) Posttranscriptional Regulatory Element (WPRE), WPRE3 (a shortened WPRE), and a functional variant (e.g., a functional truncation) thereof.

In some embodiments, the second polynucleotide sequence comprises a sequence encoding a polyadenylation (polyA) signal downstream of the sequence encoding the napRNAn.

In some embodiments, the second polynucleotide sequence comprises, downstream of the sequence encoding the napRNAn, a WPRE sequence followed by a sequence encoding a polyadenylation (polyA) signal.

In some embodiments, the polyA signal is selected from a group consisting of a bovine growth hormone polyadenylation (bGH polyA) signal, a small polyA (SPA) signal, a human growth hormone polyadenylation (hGH polyA) signal, a SV40 polyA (SV40 polyA) signal, a rabbit beta globin polyA (rBG polyA) signal, and a functional variant (e.g., a functional truncation) thereof.

In some embodiments, the polyA signal is a bGH polyA signal.

In some embodiments, the second polynucleotide sequence comprises, from 5′ to 3′, the promoter, the Kozak sequence, the first sequence encoding the first NLS, the sequence encoding the napRNAn, the second sequence encoding the second NLS, the WPRE sequence, and the sequence encoding the polyA signal.

In some embodiments, the promoter comprises, consists essentially of, or consists of a sequence encoding a polypeptide having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 47, 58, or 64; or a sequence having at most 1, 2, 3, 4, 5, 6, 7, 8, 9, or 10 nucleotide differences, whether consecutive or not, compared to the sequence of SEQ ID NO: 47, 58, or 64.

In some embodiments, the Kozak sequence comprises, consists essentially of, or consists of a sequence encoding a polypeptide having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 54; or a sequence having at most 1, 2, 3, or 4 nucleotide differences, whether consecutive or not, compared to the sequence of SEQ ID NO: 54.

In some embodiments, the NLS, the first NLS, and/or the second NLS comprises, consists essentially of, or consists of a sequence encoding a polypeptide having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 47 or 58; or a sequence having at most 1, 2, 3, 4, 5, 6, 7, 8, 9, or 10 amino acid differences, whether consecutive or not, compared to the sequence of SEQ ID NO: 47 or 58.

In some embodiments, the sequence encoding the NLS, the first sequence encoding the first NLS, and/or the second sequence encoding the second NLS comprises, consists essentially of, or consists of a sequence encoding a polypeptide having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 64 or 65; or a sequence having at most 1, 2, 3, 4, 5, 6, 7, 8, 9, or 10 amino acid differences, whether consecutive or not, compared to the sequence of SEQ ID NO: 64 or 65.

In some embodiments, the sequence encoding the polyA signal comprises, consists essentially of, or consists of a sequence encoding a polypeptide having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 48; or a sequence having at most 1, 2, 3, 4, 5, 6, 7, 8, 9, or 10 nucleotide differences, whether consecutive or not, compared to the sequence of SEQ ID NO: 48.

In some embodiments, the second polynucleotide sequence comprises, from 5′ to 3′, the promoter of SEQ ID NO: 63, the Kozak sequence of SEQ ID NO: 54, the first sequence encoding the first NLS of SEQ ID NO: 64, 5′ end truncation of the sequence encoding the napRNAn of SEQ ID NO: 62 without the first 5′ ATG codon, the second sequence encoding the second NLS of SEQ ID NO: 65, the WPRE sequence of SEQ ID NO: 59, and the sequence encoding the polyA signal of SEQ ID NO: 48.

ITR of the rAAV Vector Genome of the Disclosure

In some embodiments, the rAAV vector genome comprises a 5′ inverted terminal repeat (ITR) sequence and a 3′ ITR sequence.

In some embodiments, the 5′ ITR sequence and the 3′ ITR sequence are both wild-type ITR sequences from AAV1, AAV2, AAV3A, AAV3B, AAV4, AAV5, AAV6, AAV7, AAVrh74, AAV8, AAV9, AAV10, AAV11, AAV12, AAV13, AAV-DJ, AAV PHP.eB, or a member of the Clade to which any of the AAV1-AAV13 belong, or a functional variant (e.g., a functional truncation) thereof.

In some embodiments, the 5′ ITR sequence comprises, consists essentially of, or consists of a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 52.

In some embodiments, the 3′ ITR sequence comprises, consists essentially of, or consists of a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 53.

Configuration of the rAAV Vector Genome of the Disclosure

In some embodiments, the rAAV vector genome comprises, from 5′ to 3′, the first polynucleotide sequence, and the second polynucleotide sequence.

In some embodiments, the rAAV vector genome comprises, from 5′ to 3′, the second polynucleotide sequence, and the first polynucleotide sequence.

In some embodiments, the rAAV vector genome comprises, consists essentially of, or consists of a sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the sequence of SEQ ID NO: 56, 57, or 66.

In some embodiments, the rAAV vector genome comprises the sequence of SEQ ID NO: 66. rAAV particle of the disclosure

In yet another aspect, the disclosure provides a recombinant AAV (rAAV) particle comprising the rAAV vector genome of the disclosure. A simple introduction of AAV for delivery may refer to “Adeno-associated Virus (AAV) Guide” (www.addgene.org/guides/aav/).

Adeno-associated virus (AAV), when engineered to delivery, e.g., a protein-encoding sequence of interest, may be termed as a (r)AAV vector, a (r)AAV vector particle, or a (r)AAV particle, where “r” stands for “recombinant”. And the genome packaged in AAV vectors for delivery may be termed as a (r)AAV vector genome, vector genome, or vg for short, while viral genome may refer to the original viral genome of natural AAVs.

The serotypes of the capsids of rAAV particles can be matched to the types of target cells. For example, Table 2 of WO2018002719A1 lists exemplary cell types that can be transduced by the indicated AAV serotypes (incorporated herein by reference).

In some embodiments, the rAAV particle comprising a capsid with a serotype suitable for delivery into nerve cells (e.g., neuron). In some embodiments, the rAAV particle comprising a capsid with a serotype of AAV1, AAV2, AAV3A, AAV3B, AAV4, AAV5, AAV6, AAV7, AAVrh74, AAV8, AAV9, AAV10, AAV11, AAV12, AAV13, AAV-DJ, or AAV.PHP.eB, a member of the Clade to which any of the AAV1-AAV13 belong, or a functional variant (e.g., a functional truncation) thereof, encapsidating the rAAV vector genome. In some embodiments, the serotype of the capsid is AAV9 or AAV.PHP.eB or a mutant thereof.

General principles of rAAV particle production are known in the art. In some embodiments, rAAV particles may be produced using the triple transfection method (described in detail in U.S. Pat. No. 6,001,650).

The vector titers are usually expressed as vector genomes per ml (vg/ml). In some embodiments, the vector titer is above 1×10⁹, above 5×10¹⁰, above 1×10¹¹, above 5×10¹¹, above 1×10¹², above 5×10¹², or above 1×10¹³ vg/ml.

Instead of packaging a single strand (ss)DNA sequence as a vector genome of a rAAV particle, systems and methods of packaging an RNA sequence as a vector genome into a rAAV particle is recently developed and applicable herein. See PCT/CN2022/075366, which is incorporated herein by reference in its entirety.

When the vector genome is RNA as in, for example, PCT/CN2022/075366, for simplicity of description and claiming, sequence elements described herein for DNA vector genomes, when present in RNA vector genomes, should generally be considered to be applicable for the RNA vector genomes except that the deoxyribonucleotides in the DNA sequence are the corresponding ribonucleotides in the RNA sequence (e.g., dT is equivalent to U, and dA is equivalent to A) and/or the element in the DNA sequence is replaced with the corresponding element with a corresponding function in the RNA sequence or omitted because its function is unnecessary in the RNA sequence and/or an additional element necessary for the RNA vector genome is introduced.

As used herein, a coding sequence, e.g., as a sequence element of rAAV vector genomes herein, is construed, understood, and considered as covering and covers both a DNA coding sequence and an RNA coding sequence. When it is a DNA coding sequence, an RNA sequence can be transcribed from the DNA coding sequence, and optionally further a protein can be translated from the transcribed RNA sequence as necessary. When it is an RNA coding sequence, the RNA coding sequence per se can be a functional RNA sequence for use, or an RNA sequence can be produced from the RNA coding sequence, e.g., by RNA processing, or a protein can be translated from the RNA coding sequence.

For example, a Cas13 coding sequence encoding a Cas13 polypeptide covers either a Cas13 DNA coding sequence from which a Cas13 polypeptide is expressed (indirectly via transcription and translation) or a Cas13 RNA coding sequence from which a Cas13 polypeptide is translated (directly).

For example, a gRNA coding sequence encoding a gRNA covers either a gRNA DNA coding sequence from which a gRNA is transcribed or a gRNA RNA coding sequence (1) which per se is the functional gRNA for use, or (2) from which a gRNA is produced, e.g., by RNA processing.

In some embodiments for rAAV RNA vector genomes, 5′-ITR and/or 3′-ITR as DNA packaging signals may be unnecessary and can be omitted at least partly, while RNA packaging signals can be introduced.

In some embodiments for rAAV RNA vector genomes, a promoter to drive transcription of DNA sequences may be unnecessary and can be omitted at least partly.

In some embodiments for rAAV RNA vector genomes, a sequence encoding a polyA signal may be unnecessary and can be omitted at least partly, while a polyA tail can be introduced.

Similarly, other DNA elements of rAAV DNA vector genomes can be either omitted or replaced with corresponding RNA elements and/or additional RNA elements can be introduced, in order to adapt to the strategy of delivering an RNA vector genome by rAAV particles.

### Pharmaceutical Composition of the Disclosure

In yet another aspect, the disclosure provides a pharmaceutical composition comprising the system or composition of the disclosure or the rAAV particle of the disclosure and a pharmaceutically acceptable excipient.

In some embodiments, the pharmaceutical composition comprises the rAAV particle in a concentration selected from the group consisting of about 1×10¹⁰ vg/mL, 2×10¹⁰ vg/mL, 3×10 vg/mL, 4×10¹⁰ vg/mL, 5×10¹⁰ vg/mL, 6×10¹⁰ vg/mL, 7×10¹⁰ vg/mL, 8×10¹⁰ vg/mL, 9×10¹⁰ vg/mL, 1×10¹¹ vg/mL, 2×10¹¹ vg/mL, 3×10¹¹ vg/mL, 4×10¹¹ vg/mL, 5×10¹¹ vg/mL, 6×10¹¹ vg/mL, 7×10¹¹ vg/mL, 8×10¹¹ vg/mL, 9×10¹¹ vg/mL, 1×10¹² vg/mL, 2×10¹² vg/mL, 3×10¹² vg/mL, 4×10¹² vg/mL, 5×10¹² vg/mL, 6×10¹² vg/mL, 7×10¹² vg/mL, 8×10¹² vg/mL, 9×10¹² vg/mL, 1×10¹³ vg/mL, or in a concentration of a numerical ran e between any of two preceding values, e.g., in a concentration of from about 9×10¹⁰ vg/mL to about 8×10¹¹ vg/mL.

In some embodiments, the pharmaceutical composition is an injection.

In some embodiments, the volume of the injection is selected from the group consisting of about 1 microliter, 10 microliters, 50 microliters, 100 microliters, 150 microliters, 200 microliters, 250 microliters, 300 microliters, 350 microliters, 400 microliters, 450 microliters, 500 microliters, 550 microliters, 600 microliters, 650 microliters, 700 microliters, 750 microliters, 800 microliters, 850 microliters, 900 microliters, 950 microliters, 1000 microliters, and a volume of a numerical range between any of two preceding values, e.g., in a concentration of from about 10 microliters to about 750 microliters.

### Method of Using of the Disclosure

In yet another aspect, the disclosure provides a method for preventing or treating a disease or disorder associated with human MECP2 mRNA in a subject in need thereof, comprising administering to the subject the system or composition of the disclosure, the rAAV particle of the disclosure, or the pharmaceutical composition of the disclosure, wherein the napRNAn modifies the human MECP2 mRNA, and wherein the modification of the human MECP2 mRNA treats the disease or disorder.

In some embodiments, the disease or disorder is MECP2 Duplication Syndrome (MDS).

In some embodiments, the administrating comprises local administration or systemic administration.

In some embodiments, the administrating comprises intrathecal administration, intramuscular administration, intravenous administration, transdermal administration, intranasal administration, oral administration, mucosal administration, intraperitoneal administration, intracranial administration, intracerebroventricular administration, or stereotaxic administration.

In some embodiments, the administrating comprises intracerebroventricular administration (e.g., intracerebroventricular injection).

In some embodiments, the administration is conducted by injection.

In some embodiments, the subject is a human.

In some embodiments, the level of the human MECP2 mRNA is decreased in the subject by at least about 10%, about 15%, about 20%, about 25%, about 30%, about 35%, about 40%, about 45%, about 50%, about 55%, about 60%, about 65%, about 70%, about 75%, about 80%, about 85%, or more, compared to the level of the human MECP2 mRNA in the subject prior to the administration.

In some embodiments, the level of human MECP2 protein in the subject is at most about 10%, about 15%, about 20%, about 25%, about 30%, about 35%, about 40%, about 45%, about 50%, about 55%, about 60%, about 65%, about 70%, about 75%, about 80%, or about 85%, compared to the level of human MECP2 protein in a human or a human population not suffering from the disease or disorder.

The dose of the rAAV particle for treatment of the disease or disorder may be either via a single dose, or multiple doses. One skilled in the art understands that the actual dose may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

In some embodiments, the rAAV particle is administrated in a therapeutically effective dose. For example, the therapeutically effective dose of the rAAV particle may be about 1.0E+8, 2.0E+8, 3.0E+8, 4.0E+8, 6.0E+8, 8.0E+8, 1.0E+9, 2.0E+9, 3.0E+9, 4.0E+9, 6.0E+9, 8.0E+9, 1.0E+10, 2.0E+10, 3.0E+10, 4.0E+10, 6.0E+10, 8.0E+10, 1.0E+11, 2.0E+11, 3.0E+11, 4.0E+11, 6.0E+11, 8.0E+11, 1.0E+12, 2.0E+12, 3.0E+12, 4.0E+12, 6.0E+12, 8.0E+12, 1.0E+13, 2.0E+13, 3.0E+13, 4.0E+13, 6.0E+13, 8.0E+13, 1.0E+14, 2.0E+14, 3.0E+14, 4.0E+14, 6.0E+14, 8.0E+14, 1.0E+15, 2.0E+15, 3.0E+15, 4.0E+15, 6.0E+15, 8.0E+15, 1.0E+16, 2.0E+16, 3.0E+16, 4.0E+16, 6.0E+16, 8.0E+16, or 1.0E+17 vg, or within a range of any two of the those point values. vg stands for vector genomes of rAAV particles for administration.

### Cell

In yet another aspect, the disclosure provides a cell or a progeny thereof, comprising the guide nucleic acid of the disclosure, the system or composition or complex of the disclosure, the rAAV vector genome of the disclosure, or the rAAV particle of the disclosure. In some embodiments, the cell is a eukaryote. In some embodiments, the cell is a human cell. In some embodiments, the cell is located in the CNS of the subject. In some embodiments, the cell is a human nerve cell (e.g., neuron).

In yet another aspect, the disclosure provides a cell or a progeny thereof modified by the system or composition or complex of the disclosure, the rAAV particle of the disclosure, or the method of the disclosure. In some embodiments, the cell is a eukaryote. In some embodiments, the cell is a human cell. In some embodiments, the cell is located in the CNS of the subject. In some embodiments, the cell is a human nerve cell (e.g., neuron).

In some embodiments, the cell is not a human embryonic stem cell. In some embodiments, the cell is not a human germ cell.

### Kit

In yet another aspect, the disclosure provides a kit comprising the guide nucleic acid of the disclosure, the system or composition of the disclosure, the rAAV vector genome of the disclosure, the rAAV particle of the disclosure, or the cell or a progeny thereof of the disclosure.

In some embodiments, the kit further comprises an instruction to use the component(s) contained therein, and/or instructions for combining with additional component(s) that may be available or necessary elsewhere.

In some embodiments, the kit further comprises one or more buffers that may be used to dissolve any of the component(s) contained therein, and/or to provide suitable reaction conditions for one or more of the component(s). Such buffers may include one or more of PBS, HEPES, Tris, MOPS, Na₂CO₃, NaHCO₃, NaB, or combinations thereof. In some embodiments, the reaction condition includes a proper pH, such as a basic pH. In some embodiments, the pH is between 7-10.

In some embodiments, any one or more of the kit components may be stored in a suitable container or at a suitable temperature, e.g., 4 Celsius degree.

Further embodiments are illustrated in the following Examples which are given for illustrative purposes only and are not intended to limit the scope of the disclosure.

### The Disclosure Provides the Following Additional Embodiments

One aspect of the invention provides a recombinant adeno-associated virus (rAAV) vector genome, comprising:
(1) a Cas13 coding sequence encoding a Cas13 polypeptide,
(i) wherein said Cas13 coding sequence is at least 80%, 85%, 90%, 95%, 96%, 97%, 98%, 99%, 99.2%, 99.4%, 99.6%, 99.8%, 99.9%, or 100% identical to SEQ ID NO: 3, 5, or 62 or an RNA counterpart thereof,
(ii) wherein said Cas13 polypeptide comprises
(a) the amino acid sequence of SEQ ID NO: 2, 4, or 61, or
(b) a variant thereof that is at least 80%, 85%, 90%, 95%, 96%, 97%, 98%, or 99% identical to SEQ ID NO: 1 and has a non-conserved substitution at Y666 and/or Y677 (e.g., Y666A and/or Y677A substitution(s)) of SEQ ID NO: 1, wherein said variant has substantially the same (e.g., at least about 80%, 90%, 95%, 99% or more) guide RNA-specific nuclease activity (cleavage activity) as SEQ ID NO: 1 and substantially no (e.g., at most 20%, 15%, 10%, 5%) collateral (guide RNA-independent) nuclease activity (collateral cleavage activity) of SEQ ID NO: 1; and,
(2) a single guide RNA (sgRNA) or a sgRNA coding sequence encoding the sgRNA, said sgRNA comprises:
(A) a spacer sequence substantially complementary to a target RNA sequence on a MECP2 mRNA; and,
(B) a direct repeat (DR) sequence capable of forming a complex with said Cas13 polypeptide,
wherein the complex specifically cleaves the MECP2 mRNA at or near the target RNA sequence when said sgRNA guides said Cas13 polypeptide to the target RNA sequence;
optionally wherein the sgRNA or sgRNA coding sequence is 3′ or 5′ to the Cas13 coding sequence.

In some embodiments, the rAAV vector genome further comprises a first coding sequence for a first nuclear localization sequence (NLS, such as SEQ ID NO: 47 or 58) or nuclear export signal (NES) fused N-terminal to said Cas13 polypeptide, and/or a second coding sequence for a second NLS (such as SEQ ID NO: 47 or 58) or NES fused C-terminal to said Cas13 polypeptide;
optionally, the rAAV vector genome further comprises a coding sequence for one or more copies (e.g., 3 tandem copies) of an epitope tag, such as an HA tag, fused (e.g., C-terminally) to the Cas13 polypeptide (and the C-terminal NLS or NES, if present).

In some embodiments, the rAAV vector genome further comprises a 5′ AAV ITR sequence and a 3′ AAV ITR sequence.

In some embodiments, the 5′ and the 3′ AAV ITR sequences are both wild-type AAV ITR sequences from AAV1, AAV2, AAV3A, AAV3B, AAV4, AAV5, AAV6, AAV7, AAVrh74, AAV8, AAV9, AAV10, AAV11, AAV12, AAV13, AAV-DJ, AAV PHP.eB, or a member of the Clade to which any of the AAV1-AAV13 belong, or a functional truncated variant thereof; optionally, said 5′ AAV ITR sequence has the polynucleotide sequence of SEQ ID NO: 52, and/or said 3′ AAV ITR sequence has the polynucleotide sequence of SEQ ID NO: 53.

In some embodiments, the rAAV vector genome further comprises comprising a promoter operably linked to the Cas13 coding sequence.

In some embodiments, the promoter is a ubiquitous, tissue-specific, cell-type specific, constitutive, or inducible promoter; optionally, wherein the promoter comprises a promoter selected from the group consisting of: a Cbh promoter, a Cba promoter, a pol I promoter, a pol II promoter, a pol III promoter, a T7 promoter, a U6 promoter, a H1 promoter, a retroviral Rous sarcoma virus LTR promoter, a cytomegalovirus (CMV) promoter, a SV40 promoter, a dihydrofolate reductase promoter, a β-actin promoter, an elongation factor 1a short (EFS) promoter, a β glucuronidase (GUSB) promoter, a cytomegalovirus (CMV) immediate-early (Ie) enhancer and/or promoter, a chicken 3-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, and a myelin basic protein (MBP) promoter.

In some embodiments, the promoter comprises a Cbh promoter, such as a Cbh promoter having the polynucleotide sequence of SEQ ID NO: 44.

In some embodiments, the promoter comprises a MECP2 promoter, such as a mouse MECP2 (mMECP2) promoter having the sequence of SEQ ID NO: 63.

In some embodiments, the rAAV vector genome further comprises a polyadenylation (polyA) signal sequence, such as a bovine growth hormone polyadenylation signal (bGH polyA), a small polyA signal (SPA), a human growth hormone polyadenylation signal (hGH polyA), a SV40 polyA signal (SV40 polyA), a rabbit beta globin polyA signal (rBG polyA), and a functional truncation or variant thereof; or a corresponding polyA sequence.

In some embodiments, the polyA signal sequence comprises a bovine growth hormone polyadenylation signal (bGH polyA), or a variant thereof; optionally, said bGH polyA comprises the polynucleotide sequence of SEQ ID NO: 48.

In some embodiments, the sgRNA coding sequence is operably linked to a promoter; optionally wherein the promoter is a ubiquitous, tissue-specific, cell-type specific, constitutive, or inducible promoter; optionally selected from a group consisting of a Cbh promoter, a Cba promoter, a pol I promoter, a pol II promoter, a pot III promoter, a T7 promoter, a U6 promoter, a H1 promoter, a retroviral Rous sarcoma virus LTR promoter, a cytomegalovirus (CMV) promoter, a SV40 promoter, a dihydrofolate reductase promoter, a β-actin promoter, an elongation factor 1α short (EFS) promoter, a R glucuronidase (GUSB) promoter, a cytomegalovirus (CMV) immediate-early (Je) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, and a myelin basic protein (MBP) promoter; optionally wherein the promoter is an RNA pol III promoter or a CMV promoter (such as SEQ ID NO: 50).

In some embodiments, the RNA pot III promoter is U6 promoter, (such as SEQ ID NO: 49), H1, 7SK, or a variant thereof.

In some embodiments, (1) said sgRNA comprises one spacer sequence directly linked to one DR sequence (e.g., SEQ ID NO: 6); (2) said sgRNA comprises one spacer sequence flanked by two DR sequences (e.g., each of SEQ ID NO: 6); or (3) said sgRNA comprises two or more spacer sequences; and wherein each spacer sequence is flanked by two DR sequences each capable of forming a complex with said Cas13 polypeptide; optionally, said sgRNA comprises two spacer sequences flanked by three DR sequences to form a DR-spacer-DR-spacer-DR structure (e.g., each of SEQ ID NO: 6)
wherein each of said spacer sequence is independently substantially complementary to a distinct target RNA sequence on said MECP2 mRNA, and each capable of directing said Cas13 polypeptide to cleave respective said distinct target RNA sequence.

In some embodiments, the DR sequence comprises (1) SEQ ID NO: 6; (2) a sequence having at least 90%, 92%, 94%, 95%, 96%, 98%, or 99% identity to SEQ ID NO: 6; (3) a sequence having at most 1, 2, 3, 4, or 5 nucleotide differences from SEQ ID NO: 6; or (4) a sequence having substantially the same secondary structure as that of SEQ ID NO: 6.

In some embodiments, each said DR sequence comprises, consists essentially of, or consists of SEQ ID NO: 6.

In some embodiments, the target RNA sequence comprises a stench of contiguous nucleotides of SEQ ID NO: 60; optionally 20-50, or 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, such as 30, contiguous nucleotides of SEQ ID NO: 60, such as, any one of SEQ ID NO: 27-36.

In some embodiments, the spacer sequence is independently selected from any one of SEQ ID NOs: 17-26, or a variant thereof differing from any one of SEQ ID NOs: 17-26 by up to 1, 2, 3, 4, 5 or 6 nucleotides without substantially diminishing the ability to direct the Cas13 polypeptide to bind to the sgRNA to form a Cas13-sgRNA complex targeting the target RNA sequences to cleave the target RNA.

In some embodiments, said MECP2 mRNA is associated with a disease or disorder, such as MECP2 duplication syndrome (MDS).

In some embodiments, the rAAV vector genome comprises an ITR-to-ITR polynucleotide (such as SEQ ID NO: 56, 57, or 66) comprising, from 5′ to 3′:
(a) a 5′ ITR from AAV2 (such as SEQ ID NO: 52);
(b) a U6 or CMV promoter (such as SEQ ID NO: 49 or 50);
(c) a first direct repeat (DR) DNA coding sequence encoding a first DR (such as SEQ ID NO: 6);
(d) a first spacer coding sequence encoding a first spacer sequence specific for MECP2 mRNA (such as, any one of SEQ ID NO: 19-22 and 25);
(e) a second DR DNA coding sequence encoding a second DR (such as SEQ ID NO: 6);
(f) a second spacer coding sequence encoding a second spacer sequence specific for MECP2 mRNA (such as, any one of SEQ ID NO: 19-22 and 25);
(g) a third DR DNA coding sequence encoding a third DR (such as SEQ ID NO: 6);
(h) a Cbh promoter (e.g., one comprising an enhancer sequence (such as SEQ ID NO: 45) and an intron sequence (such as SEQ ID NO: 46); optionally, the Cbh promoter comprises SEQ ID NO: 44);
(i) a Kozak sequence (such as SEQ ID NO: 54);
(j) a first NLS coding sequence (such as one encoding SEQ ID NO: 47 or 58);
(k) a Cas13 polynucleotide (such as SEQ ID NO: 3, 5, or 62 except the start codon ATG) encoding the Cas13 polypeptide of SEQ ID NO: 2, 4, or 61 except the first amino acid M;
(l) a second NLS coding sequence (such as one encoding SEQ ID NO: 47 or 58);
(m) an optional coding sequence encoding three tandem repeats of HA tag sequences (e.g., SEQ ID NO: 55);
(n) a WPRE3 sequence (such as SEQ ID NO: 59);
(o) an bGH polyA signal sequence (such as SEQ ID NO: 48); and,
(p) a 3′ ITR from AAV2 (such as SEQ ID NO: 53);
or a polynucleotide at least about 90%, 91%, 92%, 93%, 94%, 95%, 96%, 97%, 98%, 99%, 99.5%, 99.6%, 99.7%, 99.8%, 99.9% identical to said ITR-to-ITR polynucleotide;
optionally, said ITR-to-ITR polynucleotide further comprises a linker sequence between any two adjacent sequence elements of (a)-(p);
optionally, the sequence elements of (b) to (g) that are 5′ to the sequence elements of (h) to (o) are relocated 3′ to the sequence elements of (h) to (o).

Another aspect of the invention provides a recombinant AAV (rAAV) vector genome comprising, consisting essentially of, or consisting of:
(1) SEQ ID NO: 56, 57, or 66, or a polynucleotide at least about 90%, 91%, 92%, 93%, 94%, 95%, 96%, 97%, 98%, 99%, 99.5%, 99.6%, 99.7%, 99.8%, 99.9% identical thereto, wherein said polynucleotide encodes
(a) a Cas13 polypeptide of SEQ ID NO: 2, 4, or 61, or
(b) a variant thereof at least 80%, 85%, 90%, 95%, 96%, 97%, 98%, or 99% identical to SEQ ID NO: 1 and having a non-conserved substitution at Y666 and/or Y677 (e.g., Y666A and/or Y677A substitution(s)) of SEQ ID NO: 1, wherein said variant has substantially the same (e.g., at least about 80%, 90%, 95%, 99% or more) guide RNA-specific nuclease activity as SEQ ID NO: 1 and substantially no (e.g., at most 20%, 15%, 10%, 5%) collateral (guide RNA-independent) nuclease activity of SEQ ID NO: 1; and,
(2) a sg RNA coding sequence encoding a sgRNA, said sgRNA comprises:
(A) a spacer sequence substantially complementary to a target RNA sequence on a MECP2 mRNA; and,
(B) a direct repeat (DR) sequence that forms a complex with said Cas13 polypeptide,
wherein the complex specifically cleaves the MECP2 mRNA with substantially the same (e.g., at least about 80%, 90%, 95%, 99% or more) guide RNA-specific nuclease activity as SEQ ID NO: 1 and substantially no (e.g., at most 20%, 15%, 10%, 5%) collateral (guide RNA-independent) nuclease activity of SEQ ID NO: 1,
at or near the target RNA sequence when said sgRNA guides said Cas13 polypeptide to the target RNA sequence; optionally wherein the sgRNA coding sequence is 3′ or 5′ to the Cas13 coding sequence.

In some embodiments, the rAAV vector genome is SEQ ID NO: 56, 57, or 66, or the polynucleotide at least 95% or 99% identical thereto.

Another aspect of the invention provides a recombinant AAV (rAAV) viral particle comprising the rAAV vector genome as described herein.

In some embodiments, the rAAV viral particle comprises a capsid with a serotype of AAV1, AAV2, AAV3A, AAV3B, AAV4, AAV5, AAV6, AAV7, AAVrh74, AAV8, AAV9, AAV10, AAV11, AAV12, AAV13, AAV-DJ, or AAV PHP.eB, a member of the Clade to which any of the AAV1-AAV13 belong, or a functional truncated variant or a functional mutant thereof, encapsidating the rAAV vector genome.

In some embodiments, the capsid serotype is AAV PHP.eB or AAV9.

Another aspect of the invention provides a recombinant AAV (rAAV) viral particle comprising the rAAV vector genome as described herein, encapsidated in a capsid with a serotype of AAV PHP.eB or AAV9.

Another aspect of the invention provides a pharmaceutical composition comprising the rAAV vector genome as described herein, or the rAAV viral particle as described herein, and a pharmaceutically acceptable excipient.

Another aspect of the invention provides a method of treating a disease or disorder associated with MECP2 in a subject in need thereof, the method comprising administering to the subject a therapeutically effective amount of the rAAV vector genome as described herein, the rAAV viral particle as described herein, or the pharmaceutically composition as described herein, wherein the rAAV vector genome or the rAAV viral particle specifically down-regulate the expression of said MECP2 causative of the disease or disorder.

In some embodiments, the administrating comprises contacting a cell with the therapeutically effective amount of the rAAV vector genome as described herein, the rAAV viral particle as described herein, or the pharmaceutically composition as described herein.

In some embodiments, the cell is located in the CNS of the subject.

In some embodiments, the disease or disorder is MECP2 duplication syndrome (MDS).

In some embodiments, the administrating comprises stereotactic or intracerebroventricular administration.

In some embodiments, the subject is a human.

In some embodiments, the expression of MECP2 in the cell is decreased in comparison to a cell having not been contacted with the rAAV vector genome as described herein, the rAAV viral particle as described herein, or the pharmaceutically composition as described herein.

In some embodiments, the expression of MECP2 is decreased in the subject by about 30%, about 35%, about 40%, about 45%, about 50%, about 55%, about 60%, about 65%, about 70%, about 75%, about 80%, or about 85% compared to the expression of MECP2 in the subject prior to administration.

Another aspect of the invention provides a guide RNA (gRNA) (e.g., a single guide RNA) comprising:
(A) a spacer sequence substantially complementary to a target RNA sequence on a MECP2 mRNA; and,
(B) optionally, a direct repeat (DR) sequence capable of forming a complex with a Cas13 polypeptide, wherein the complex specifically cleaves the MECP2 mRNA at or near the target RNA sequence when said sgRNA guides said Cas13 polypeptide to the target RNA sequence.

In some embodiments, the target RNA sequence comprises a stench of contiguous nucleotides of SEQ ID NO: 60; optionally 20-50, or 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, such as, 30 contiguous nucleotides of SEQ ID NO: 60, such as, any one of SEQ ID NO: 27-36.

In some embodiments, the gRNA comprises two or more identical or different spacer sequences, each flanked by two said DR sequence.

In some embodiments, the gRNA comprises two different spacer sequences (e.g., spacer 1 and spacer 2) separating three of said DR sequences (e.g., DR-spacer 1-DR-spacer 2-DR).

In some embodiments, the gRNA comprises one or more spacer sequences each independently selected from any one of SEQ ID NOs: 17-26, or a variant thereof differing from any one of SEQ ID NOs: 17-26 by up to 1, 2, 3, 4, 5 or 6 nucleotides without substantially diminishing the ability to direct the Cas13 polypeptide to bind to the sgRNA to form a Cas13-sgRNA complex targeting the respective target sequences to cleave the target sequences.

In some embodiments, the DR sequence comprises (1) SEQ ID NO: 6; (2) a sequence having at least 90%, 92%, 94%, 95%, 96%, 98%, or 99% identity to SEQ ID NO: 6; (3) a sequence having at most 1, 2, 3, 4, or 5 nucleotide differences from SEQ ID NO: 6; or (4) a sequence having substantially the same secondary structure as that of SEQ ID NO: 6.

In some embodiments, the Cas13 polypeptide comprises
(a) the amino acid sequence of SEQ ID NO: 2, 4, or 61, or
(b) a variant thereof that is at least 80%, 85%, 90%, 95%, 96%, 97%, 98%, or 99% identical to SEQ ID NO: 1 and has a non-conserved substitution at Y666 and/or Y677 (e.g., Y666A and/or Y677A substitution(s)) of SEQ ID NO: 1, wherein said variant has substantially the same (e.g., at least about 80%, 90%, 95%, 99% or more) guide RNA-specific nuclease activity as SEQ ID NO: 1 and substantially no (e.g., at most 20%, 15%, 10%, 5%) collateral (guide RNA-independent) nuclease activity of SEQ ID NO: 1.

Another aspect of the invention provides a cell or a progeny thereof, comprising the AAV vector genome as described herein, the rAAV viral particle as described herein, or the gRNA as described herein.

Another aspect of the invention provides a kit comprising the AAV vector genome as described herein, the rAAV viral particle as described herein, the gRNA as described herein, or the cell or a progeny thereof as described herein.

Another aspect of the invention provides a method of preparing the rAAV particle as described herein, the method comprising:
a) introducing into an AAV packaging system a nucleic acid encoding the rAAV vector genome as described herein or the RNA sequence transcribed therefrom, and a coding sequence for the AAV capsid as described herein, for a period of time sufficient to package the rAAV vector genome or the transcribed RNA into the AAV capsid to produce the rAAV particle, and
b) harvesting the rAAV particle; and, optionally,
c) isolating or purifying the harvested rAAV particle.

### EXAMPLES

The following examples are provided to further illustrate some embodiments of the disclosure but are not intended to limit the scope of the invention; it will be understood by their exemplary nature that other procedures, methodologies, or techniques known to those skilled in the art may alternatively be used.

### Example 1 High Efficiency In Vitro Knockdown of MECP2 Expression by CRISPR-hfCas13f v2 System

This example demonstrates the high in vitro knockdown efficiency of MECP2 expression by the subject CRISPR-hfCas13f v2 system.

### Plasmid Construction:

The subject CRISPR-hfCas13f v2 system comprised a hfCas13f v2 protein (SEQ ID NO: 2, Cas13f.1-Y666A, Y677A mutant) and a MECP2-targeting sgRNA (one of “sgRNA[MECP2]1-10” or “sg1-10” hereinafter, SEQ ID NO: SEQ ID NO: 7-16) comprising two Direct Repeats (each of SEQ ID NO: 6) and a Spacer (one of Spacer 1-10, SEQ ID NO: 17-26) targeting the target sequence (one of target RNA sequences 1-10, SEQ ID NO: 27-36) of MECP2 mRNA. As a negative control, a non-targeting-sgRNA (“sgRNA[NT]” or “NT” hereinafter, SEQ ID NO: 41) comprising two Direct Repeats (each of SEQ ID NO: 6) and a non-targeting-Spacer (SEQ ID NO: 42) was used in place of the sgRNA[MECP2].

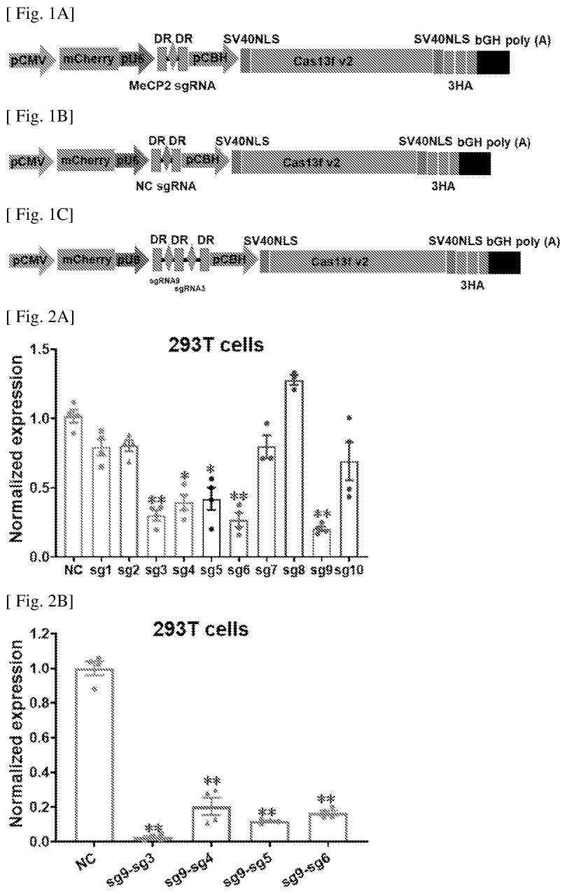

The hfCas13f v2 protein (SEQ ID NO: 2) and the sgRNA[MECP2](SEQ ID NO: 7-16) or the sgRNA[NT](SEQ ID NO: 41) were encoded together with a mCherry reporter (SEQ ID NO: 51) into the same plasmid to form a treatment or control plasmid as shown in FIGS. 1A and 1B.

The Spacers were also used in combinations. The hfCas13f v2 protein (SEQ ID NO: 2) and a sgRNA[MECP2] containing two Spacers (9 & 3, 9 & 4, 9 & 5, or 9 & 6, SEQ ID NO: 37-40) were encoded together with a mCherry reporter (SEQ ID NO: 51) into the same plasmid construct as shown in FIG. 1C.

### Plasmid Transfection:

HEK293T cells were cultured in Dulbecco's modified eagle medium (DMEM) containing 10% fetal bovine serum (FBS) and penicillin/streptomycin, and maintained at 37° C. with 5% CO₂. The cultured cells were then seeded onto 6-well plates (1.0-1.2E+6 cells/well) and transfected with 3 μg treatment or control plasmid using polyethylenimine (PEI) transfection reagent. mCherry positive transfected cells were sorted out for RNA extraction using flow cytometry 2 days after transfection.

African green monkey kidney fibroblast-like cell line Cos-7 cells as a typical target model cell for studying MECP2 gene were cultured in Dulbecco's modified eagle medium (DMEM) containing 10% fetal bovine serum (FBS) and penicillin/streptomycin, and maintained at 37° C. with 5% CO₂. The cultured cells were then seeded onto 6-well plates (1.0-1.2E+6 cells/well) and transfected with 3 μg treatment or control plasmid using polyethylenimine (PEI) transfection reagent. mCherry positive transfected cells were sorted out for RNA extraction using flow cytometry 2 days after transfection.

### RT-qPCR Detection:

Total RNA from the RNA extraction was first purified using Trizol (Ambion) and then reverse transcribed into complementary DNA (HiScript Q RT SuperMix for qPCR, Vazyme, Biotech) for RT-qPCR detection. RT-qPCR reactions were tracked by SYBR green (AceQ qPCR SYBR Green Master Mix, Vazyme, Biotech). The transcription level of MECP2 mRNA was measured and normalized to the mRNA levels of a housekeeping gene ACTIN using MECP2 primers and hACTIN primers.

### Results:

To determine whether CRISPR-hfCas13f v2 system could be used to reduce the expression of MECP2 gene, 10 single-guide (sg) RNAs (with the homology in human, mice, and monkey) targeting MECP2 mRNA were designed.

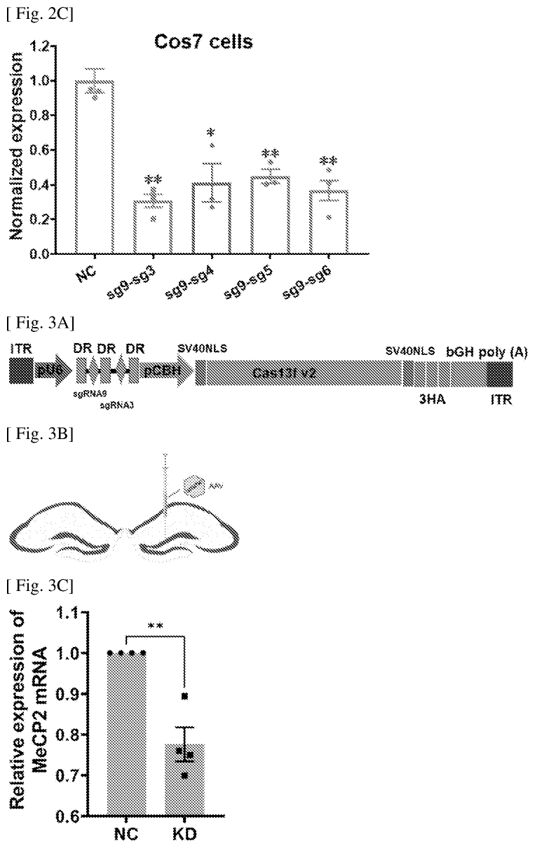

With a single Spacer used, MECP2-targeting CRISPR-hfCas13f v2 systems with sg3, 4, 5, 6, and 9 decreased MECP2 mRNA level by >about 60% relative to the negative control (FIG. 2A and Table 1).

With dual-Spacer used, the MECP2-targeting CRISPR-hfCas13f v2 system with paired Spacers 9 & 3, 9 & 4, 9 & 5, and 9 & 6 decreased MECP2 mRNA level by >about 80% relative to the negative control in 293T cells (FIG. 2B and Table 2), with optimal 97.42% for sgRNA9 & 3, and by >about 55% relative to the negative control in Cos7 cells (FIG. 2C and Table 3), with optimal 69.23% for sgRNA9 & 3.

All the results indicated great potential for the treatment of MECP2-associated diseases with the subject MECP2-targeting CRISPR-hfCas13fv2 system.

TABLE 1
Decreased MECP2 mRNA level for each sgRNA in 293T cell

| | | | | Decreased MECP2 |
|---|---|---|---|---|
| sgRNA | N | mean | ±SEM | mRNA level |
| NC | 4 | 1.017 | 0.04599 | NA |
| sg1 | 4 | 0.7907 | 0.05794 | 0.2093 |
| sg2 | 4 | 0.8029 | 0.04069 | 0.1971 |
| sg3 | 4 | 0.3002 | 0.03816 | 0.6998 |
| sg4 | 4 | 0.3945 | 0.056 | 0.6055 |
| sg5 | 4 | 0.4201 | 0.07952 | 0.5799 |
| sg6 | 4 | 0.2665 | 0.05025 | 0.7335 |
| sg7 | 3 | 0.7961 | 0.08442 | 0.2039 |
| sg8 | 3 | 1.277 | 0.03631 | −0.277 |
| sg9 | 4 | 0.2025 | 0.01747 | 0.7975 |
| sg10 | 4 | 0.6905 | 0.1363 | 0.3095 |

TABLE 2
Decreased MECP2 mRNA level for sgRNA
with dual-Spacers in 293T cell

| | | | | Decreased MECP2 |
|---|---|---|---|---|
| sgRNA | N | mean | ±SEM | mRNA level |
| NC | 4 | 1 | 0.04045 | NA |
| sgRNA9&3 | 4 | 0.02584 | 0.008001 | 0.97416 |
| sgRNA9&4 | 4 | 0.2046 | 0.04903 | 0.7954 |
| sgRNA9&5 | 4 | 0.1175 | 0.005741 | 0.8825 |
| sgRNA9&6 | 4 | 0.1658 | 0.01372 | 0.8342 |

TABLE 3
Decreased MECP2 mRNA level for sgRNA
with dual sgRNAs in Cos7 cell

| | | | | Decreased MECP2 |
|---|---|---|---|---|
| sgRNA | N | mean | ±SEM | mRNA level |
| NC | 4 | 1 | 0.06979 | NA |
| sgRNA9&3 | 4 | 0.3077 | 0.03665 | 0.6923 |
| sgRNA9&4 | 4 | 0.411 | 0.1112 | 0.589 |
| sgRNA9&5 | 4 | 0.4499 | 0.04057 | 0.5501 |
| sgRNA9&6 | 4 | 0.3675 | 0.05807 | 0.6325 |

### Example 2 High Efficiency In Vivo Knockdown of MECP2 Expression by CRISPR-hfCas13f v2 System

To demonstrate the in vivo editing efficiency of the subject CRISPR-hfCas13f v2 system in Example 1, AAV.PHP.eB delivery system was used to delivery in vivo the system to target cells in animals.

### AAVPHP.eB Transgene Plasmid Construction:

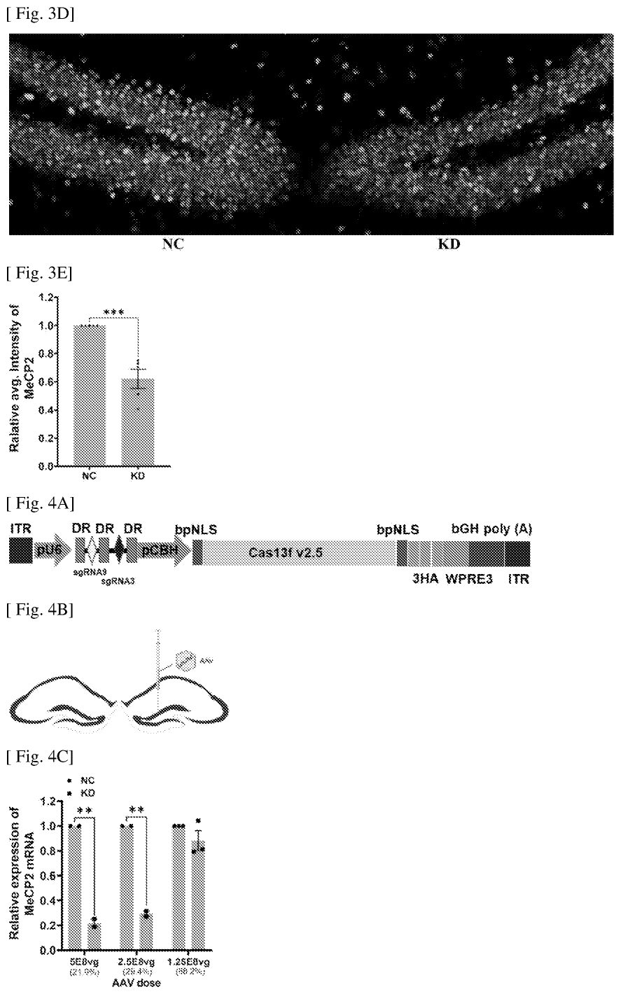

Similar to the treatment plasmids in Example 1, a treatment transgene plasmid for AAV.PHP.eB packaging encoding the hfCas13f v2 and the sgRNA [MECP2]9 & 3 were constructed as shown in FIG. 3A.

### Recombinant AAV.PHP.eB Particles Preparation:

The treatment AAV.PHP.eB particles herein were produced using conventional triple-plasmid transfection system mutatis mutandis, by co-transfecting the respective transgene plasmids, packaging plasmids, and helper plasmids in a weight ratio of 1:1:2 into HEK293T cells. The transgene plasmids were packaged by AAV.PHP.eB capsids to form the genomes inside the capsids, and together the genome and the capids constituted the AAV.PHP.eB particles.

Specifically, the HEK293T cells were cultured in competent DMEM medium, and the cells were plated 24 hrs before transfection of the plasmids. Shortly before transfection, the culture medium was replaced with fresh DMEM containing 2% FBS. PEI-MAX was used as the transfection reagent. The transfected HEK293T cells were harvested from the media at 72 hours post translation. The treatment AAV.PHP.eB particles were purified from the cells by using iodixanol density gradient ultracentrifugation.

RT-qPCR was used with a pair of hfCas13f v2 primers specific for the hfCas13f v2 sequence on the genomes to detect the genome titre of any genomes packaged in the treatment AAV.PHP.eB particles.

### Mice:

C57BL/6 mice were purchased from Beijing Vital River Laboratory Animal Technology Co., Ltd. All experimental protocols were approved by the Animal Care and Use Committee of the Institute of Neuroscience, Chinese Academy of Sciences, Shanghai, China and HUIGENE THERAPEUTICS CO., LTD.

Unilateral Stereotactic Injection into Hippocampus (Hip) Dentate Gyrus (DG) Region of C57BL/6 Mice to Verify the Knockdown Effect In Vivo:

8 weeks old C57BL/6 mice were anesthetized with a mixture of zoletil (60 μg/g) and xylazine (10 μg/g), and then unilaterally stereotactically injected with 500 nl of a AAV.PHP.eB injection (FIG. 3B) in a dose of 5E+8 vg/mice (1E+13 vg/mL) in the Hip DG region (x=−1.15, y=−1.7, and z=−2.15).

### RT-qPCR Detection:

3 weeks after the injection, the mice were anesthetized and perfused with PBS, and Hip tissues of those C57BL/6 mice were harvested. Total RNA of Hip was extracted and purified with Trizol (Ambion) and then reverse transcribed into complementary DNA (HiScript Q RT SuperMix for qPCR, Vazyme, Biotech) for RT-qPCR. The RNA levels of mMECP2 were detected with RT-qPCR by SYBR green probe (AceQ qPCR SYBR Green Master Mix, Vazyme, Biotech) and normalized to the levels of a housekeeping gene mACTIN mRNA using MECP2 primers and mACTIN primers.

### Immunofluorescence (IF) Detection:

3 weeks after the injection, the mice were anesthetized and perfused with PBS solution and subsequent 4% paraformaldehyde (PFA) solution. The whole brains of the mice were harvested and fixed in a 4% PFA solution. After 8 h, the brains were washed with PBS and dehydrated using 30% sucrose until the tissues were completely covered. The brains were embedded in OCT medium (SAKURA) and cut into 35 μm-thick sections using a cryostat.

The brain sections were washed for 5 min with PBS, incubated in 0.01 M citrate buffer at pH 6 and at 70° C. for 30 min and then cooled for 30 min at RT, blocked with blocking solution for 1 h and then incubated in primary antibody solution overnight at 4° C. Primary antibody against the following antigen was used: MECP2 (1:1,000; CST, Catalog #3456s). Further incubation with a secondary antibody (Alexa 647-conjugated; 1:1,000; Thermo Scientific) for the detection of the primary antibody was conducted at RT for 2 h, followed by nuclear counterstaining with DAPI (Invitrogen; Catalog #P36931). Fluorescent confocal slices were observed using a Nikon C2+ confocal microscope. The images were analyzed using Image J.

### Results

The in vivo knockdown efficiency of the subject CRISPR-hfCas13f v2 system with sg9 & 3 was 22.4% for MECP2 mRNA (FIG. 3C) and 37.9% for MECP2 protein (FIGS. 3D and 3E) as compared with the uninjected side of the same Hip as a negative control, indicating great efficiency in Hip for the treatment of MeCP2-associated diseases.

### Example 3 High Efficiency In Vivo Knockdown of MECP2 Expression by CRISPR-hfCas13f v2.5 System

To demonstrate the in vivo editing efficiency of the subject CRISPR-hfCas13f v2.5 system, AAV.PHP.eB delivery system was used to delivery in vivo the system to target cells in animals. CRISPR-hfCas13f v2.5 system differs from CRISPR-hfCas13f v2 system in Examples 1 and 2 in the use of hgCas13f v2.5 (SEQ ID NO: 4, Cas13f.1-L641A, Y666A, Y677A mutant) in place of hfCas13f v2 (SEQ ID NO: 2, Cas13f.1-Y666A, Y677A mutant).

### AAVPHP.eB Transgene Plasmid Construction:

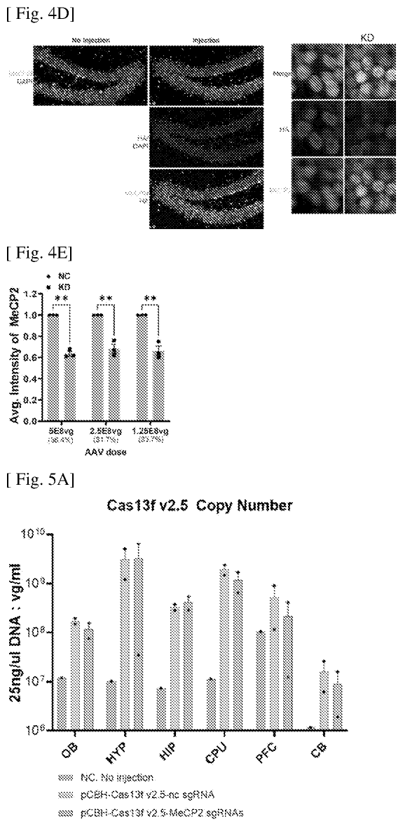

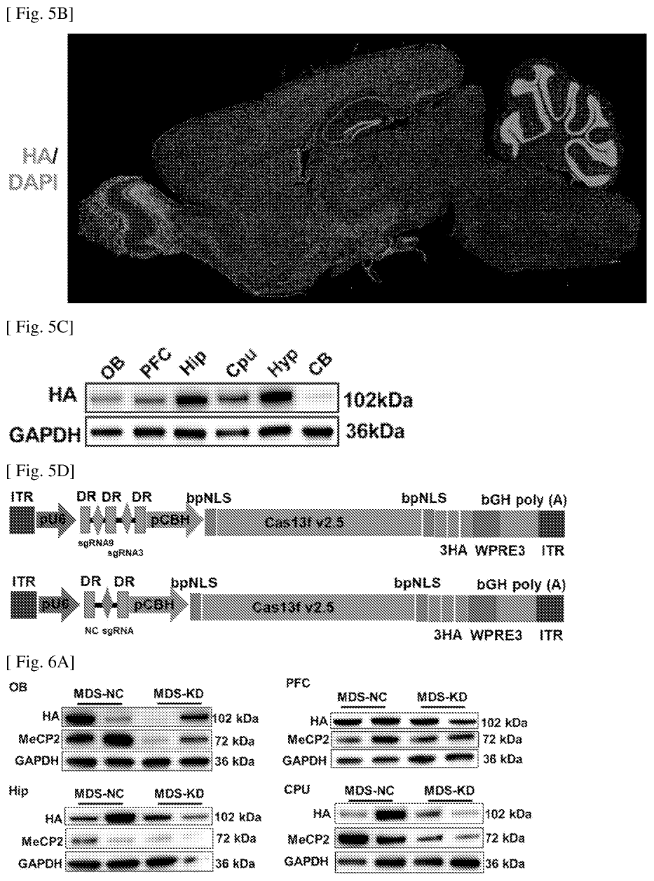

Similar to the treatment and control plasmids in Example 1, treatment and control transgene plasmids for AAV.PHP.eB packaging encoding the hfCas13f v2.5 and the sgRNA[MECP2]9 & 3 or sgRNA[NT] were constructed, respectively, as shown in FIGS. 4A and 5D.

### Recombinant AAVPHPeB Particles Preparation:

Both the treatment and control AAV.PHP.eB particles herein were produced using conventional triple-plasmid transfection system mutatis mutandis, by co-transfecting the respective transgene plasmids, packaging plasmids, and helper plasmids in a weight ratio of 1:1:2 into HEK293T cells. The transgene plasmids were packaged by AAV.PHP.eB capsids to form the genomes inside the capsids, and together the genome and the capids constituted the AAV.PHP.eB particles.

Specifically, the HEK293T cells were cultured in competent DMEM medium, and the cells were plated 24 hrs before transfection of the plasmids. Shortly before transfection, the culture medium was replaced with fresh DMEM containing 2% FBS. PEI-MAX was used as the transfection reagent. The transfected HEK293T cells were harvested from the media at 72 hours post translation. The treatment and control AAV.PHP.eB particles were purified from the cells by using iodixanol density gradient ultracentrifugation.

RT-qPCR was used with same pair of hfCas13f v2 primers in Example 2 specific for the hfCas13f v2.5 sequence on the genomes to detect the genome titre of any genomes packaged in the treatment and control AAV.PHP.eB particles.

### Mice:

C57BL/6 mice were purchased from Beijing Vital River Laboratory Animal Technology Co., Ltd., and FVB/N-Tg (MECP2/EGFP)1Hzo/J (MDS) mice were purchased from Jax lab and housed in the in-house animal facility on 12 h:12 h light/dark cycle with food and water ad libitum. P0-P2 (postnatal day 0-2) MDS mice were produced by crossing FVB/N-Tg (MECP2/EGFP)1Hzo/J mice, and the genotypes were determined by PCR using MDS genotype test primers and positive control primers. All experimental protocols were approved by the Animal Care and Use Committee of the Institute of Neuroscience, Chinese Academy of Sciences, Shanghai, China and HUIGENE THERAPEUTICS CO., LTD.

Unilateral Stereotactic Injection into Hippocampus (Hip) Dentate Gyrus (DG) Region of C57BL/6 Mice to Verify the Knockdown Effect In Vivo:

8 weeks old C57BL/6 mice were anesthetized with a mixture of zoletil (60 μg/g) and xylazine (10 μg/g), and then unilaterally stereotactically injected with 500 nl of a AAV-PHP.eB injection (FIG. 4B) with different doses of 5E+8, 2.5E+8, or 1.25E+8 vg/mice in the Hip DG region (x=−1.15, y=−1.7, and z=−2.15).

### Intracerebroventricular (ICV) Administration to Brains of MDS Mice for Treatment:

P2 MDS mice were anesthetized on the ice for 3 minutes. A small hole at slight posterior to the limbus were punctured with a sterile 31 G ½ needle following pupil dilation. 4 μL treatment or control AAV.PHP.eB injection with dose of 1E+13vg/mL (4E+10vg/mouse) was injected through the hole using a Hamilton syringe with a 33G blunt needle.

### RT-qPCR Detection:

3 weeks after the injection, the mice were anesthetized and perfused with PBS, and Hip tissues of C57BL/6 mice were harvested. Total RNA of Hip was extracted and purified with Trizol (Ambion) and then reverse transcribed into complementary DNA (HiScript Q RT SuperMix for qPCR, Vazyme, Biotech) for RT-qPCR. The RNA levels of mMECP2 were detected with RT-qPCR by SYBR green probe (AceQ qPCR SYBR Green Master Mix, Vazyme, Biotech) and normalized to the levels of a housekeeping gene mACTIN mRNA using MECP2 primers and mACTIN primers.

### Western Blot Detection:

3 weeks after the injection, the mice were anesthetized and perfused with PBS, Hip tissues of C57BL/6 mice and prefrontal cortex (PFC), hip, striatum (CPU), hypothalamus (Hyp), olfactory bulb (OB), and cerebellum (CB) tissues of MDS mice were harvested. Total protein of the above tissues was extracted with RIPA lysis buffer (Beyotime; Catalog #P0013B), and the protein concentration was determined using the bicinchoninic acid (BCA) method (Thermo Fisher; Catalog #23225). Equal amounts (20 μg) of total protein from each sample were separated by 4-20% SDS-polyacrylamide gel electrophoresis, and each aliquot was then transferred to Immobilon-PSQ Transfer Membranes (Millipore; Catalog #ISEQ00010). The membranes were incubated in blocking solution (5% nonfat dry milk in TBS) at room temperature (RT) for 1 h. Incubation with the primary antibodies [including antibodies against MECP2 (1:500; CST, Catalog #3456s), HA (1:500; CST; Catalog #3724), and GAPDH (1:1,000; Proteintech; Catalog #60004-1-Ig), as loading control] was performed at 4° C. overnight. Further incubation with the secondary antibody (Anti-Rabbit IgG HRP linked Antibody and Anti-Mouse IgG HRP linked Antibody, CST, Catalog #7074S and 70765, dilution of 1:3,000 in TBST buffer) for the detection of the primary antibodies was conducted for 1-2 h at RT. Images were captured using ECL (Beyotime; Catalog #P0018AS). The gray value of MECP2 was quantified by using ImageJ, and the relative expression level of MECP2 was estimated as the gray value of MECP2/the gray value of the loading control GAPDH.

### Immunofluorescence (IF) Detection:

3 weeks after the injection, the mice were anesthetized and perfused with 4% paraformaldehyde (PFA) solution. The whole brains of the mice were harvested and fixed in a 4% PFA solution. After 8 h, the brains were washed with PBS and dehydrated using 30% sucrose until the tissues were completely covered. The brains were embedded in OCT medium (SAKURA) and cut into 35 μm-thick sections using a cryostat.

The brain sections were washed for 5 min with PBS, incubated in 0.01 M citrate buffer at pH 6 and at 70′C for 30 min and then cooled for 30 min at RT, blocked with blocking solution for 1 h and then incubated in primary antibody solution overnight at 4° C. Primary antibodies against the following antigens were used: MECP2 (1:1,000; CST, Catalog #3456s), HA (1:500; Roche, Catalog #11867423001), and NeuN (1:1,000; CST, Catalog #24307s). Further incubation with the secondary antibodies (Alexa 546-, and 647-conjugated; 1:1,000; Thermo Scientific) for the detection of the primary antibodies was conducted at RT for 2 h, followed by nuclear counterstaining with DAPI (Invitrogen; Catalog #P36931). Fluorescent confocal slices were observed using a Nikon C2+ confocal microscope. The images were analyzed using Image J.

### Behavior Test:

All data acquisition and analyses were carried out by an individual blinded to the genotype and treatment. All behavioral studies were performed during the light period. Mice were habituated to the test room for 1 h before each test. At least one day was given between assays for the mice to recover.

Accelerating rotarod test. After habituation in the test room, motor coordination was measured using an accelerating rotarod apparatus (Ugo Basile). Mice were tested for two consecutive days, three trials each, with an interval of 60 min between trials to rest. Each trial lasted for a maximum of 10 min, and the rod accelerated from 4 to 40 r.p.m. in the first 5 min. The time that it took for each mouse to fall from the rod (latency to fall) was recorded.

Open field test. After habituation in the test room, mice were placed in the center of an open arena (40×40×30 cm), and their behavior was tracked by laser photobeam breaks for 10 min. General locomotor activity was automatically analyzed using EthoVision XT software (Noldus). In addition, rearing activity, the time spent in the center of the arena and entries to the center, were analyzed.

Elevated plus maze test. After habituation in the test room, mice were placed in the center part of the maze facing one of the two open arms. Mouse behavior was video-tracked for 10 min, and the time mice spent in the open arms and the entries to the open arms, as well as the distance travelled in the open arms, were recorded and analyzed using EthoVision XT software (Noldus).

Three-chamber test. Age- and gender-matched C57Bl/6 mice were used as novel partners. Two days before the test, the novel partner mice were habituated to the wire cups (3 inches diameter by 4 inches in height) for 1 h per day. After habituation in the test room, mice were placed in the central chamber and allowed to explore the three chambers for 10 min. Next, a novel partner mouse was placed into a wire cup in either the left or the right chamber. An inanimate object was placed as control in the wire cup of the opposite chamber. The location of the novel mouse was randomized between left and right chambers across subjects to control for side preference. The mouse tested was allowed to explore again for an additional 10 min. The time spent investigating the novel partner (defined by rearing, sniffing or pawing at the wire cup) and the time spent investigating the inanimate object were analyzed using EthoVision XT software (Noldus) were recorded.

### Results

The in vivo knockdown efficiency of the subject CRISPR-hfCas13f v2.5 system with sg9 & 3 was 78.1% (5E+8 vg/mice), 70.6% (2.5E+8 vg/mice), and 11.8% (1.25E+8 vg/mice) for MECP2 mRNA (FIG. 4C) and 36.4% (5E+8 vg/mice), 31.7% (2.5E+8 vg/mice), and 33.7% (1.25E+8 vg/mice) for MECP2 protein (FIGS. 4D and 4E) with unilateral stereotactic injection of AAV.PHP.eB particles into the Hip DG region of C57BL/6 mice, as compared with the uninjected side of the same Hip as a negative control, indicating great efficiency in Hip for the treatment of MeCP2-associated diseases.

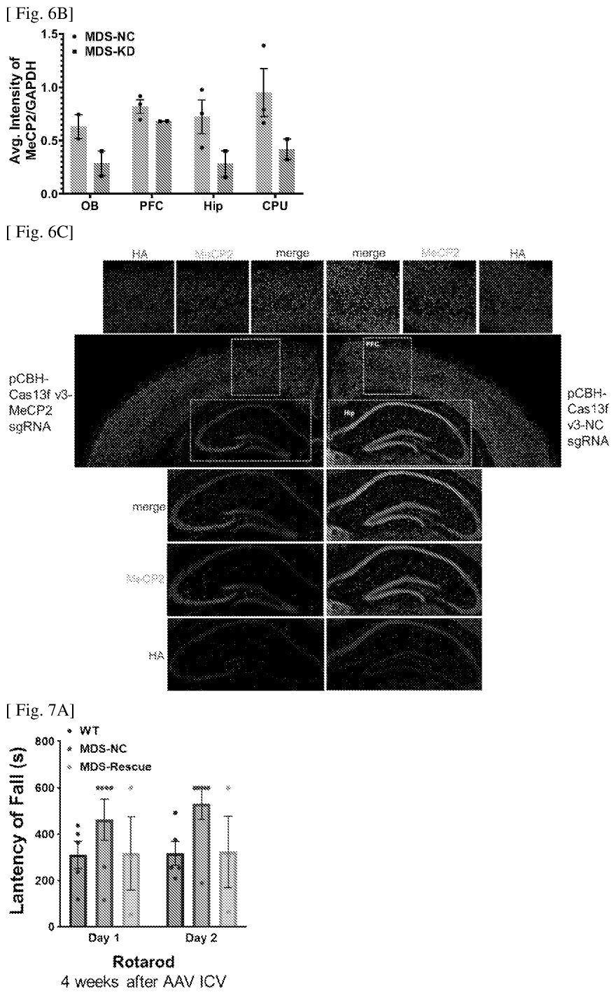

The RT-qPCR results for hfCas13f v2.5 (FIG. 5A) and the IF (FIG. 5B) and WB (FIG. 5C) results for HA-tag in the AAV vector genome showed that the ICV administration of the AAV.PHP.eB particles successfully delivered the AAV vector genomes to the whole brain of MDS mice and hfCas13f v2.5 was successfully expressed. The in vivo knockdown efficiency of MECP2 protein by the subject CRISPR-hfCas13f v2.5 system in a dose of 4E+10 vg/mouse was about 67% in Hip, 62% in CPU, 55% in OB, and 15% in PFC as compared with the negative control with ICV injection in whole brain of control AAV.PHP.eB particles (FIGS. 6A, 6B, and 6C), indicating great efficiency in the whole brain for the treatment of MECP2-associated diseases.

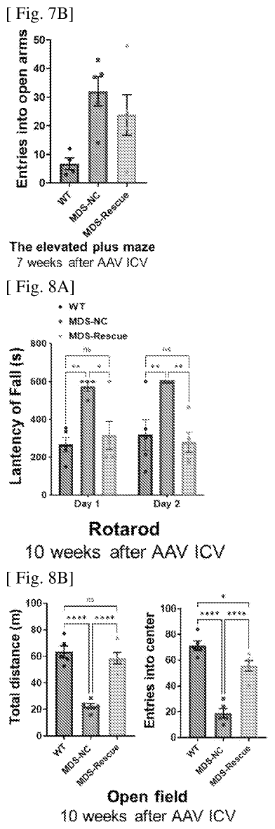

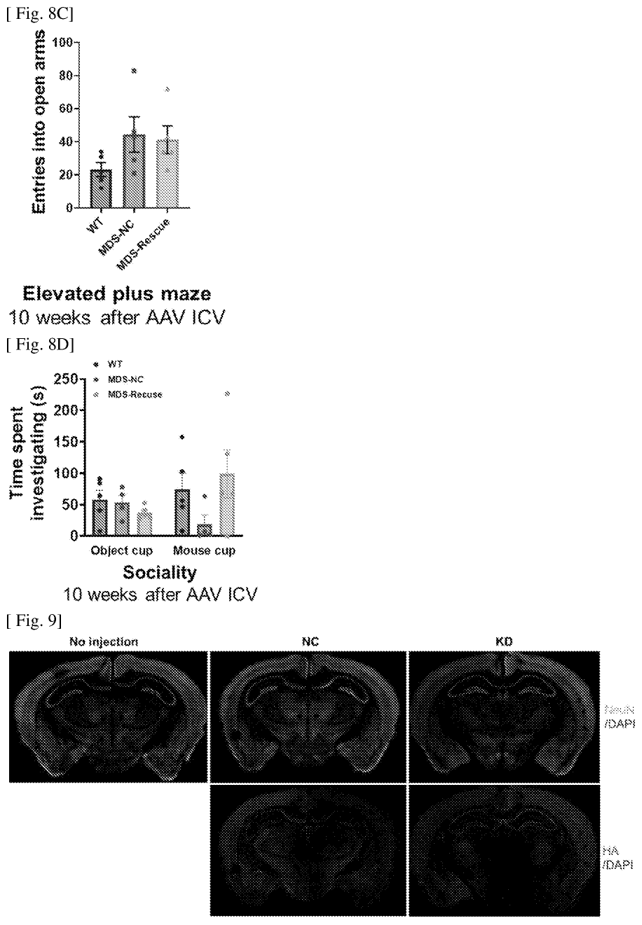

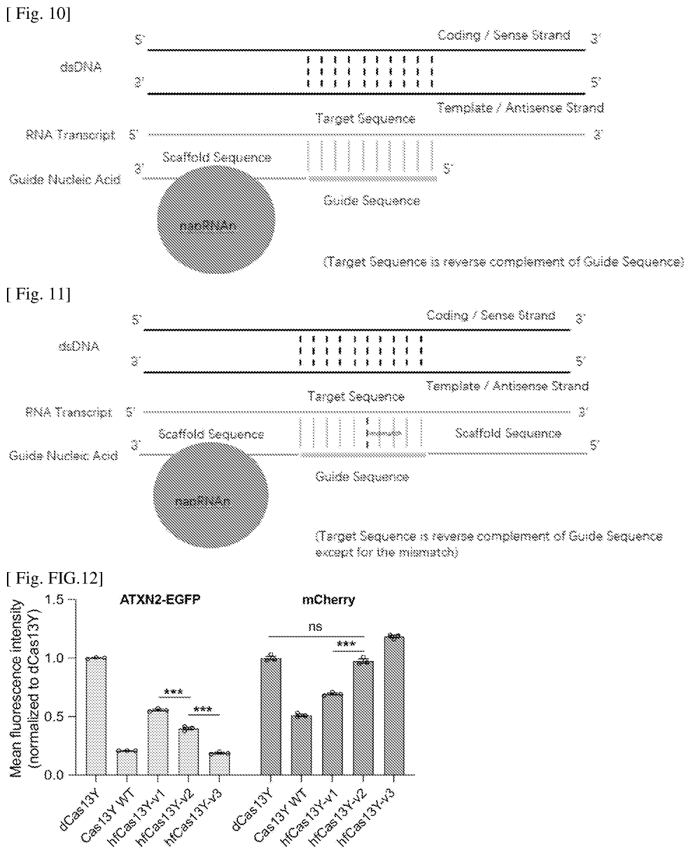

The behaviors of MDS mice injected with the AAV.PHP.eB particles were separately assessed at Week 4 for the accelerating rotarod test, Week 7 for the elevated plus maze test, and Week 10 for the accelerating rotarod test, the open field test, the elevated plus maze test, and the three-chamber test after ICV administration. The results showed the abnormal motor behavior in the accelerating rotarod test of MDS mice was partly reversed at Week 4 after AAV ICV administration (FIG. 7A), and the anxiety-like behavior in the elevated plus maze test of MDS mice was partly reversed at Week 7 after AAV ICV administration (FIG. 7B). At Week 10 after AAV ICV administration, the abnormal motor in the accelerating rotarod test, the anxiety-like behavior in the open field test and the elevated plus maze test, and the social behavior in the three-chamber test of MDS mice were almost entirely reversed (FIG. 8A-8D), indicating the abnormal behaviors of the MDS mice were rescued by the subject CRISPR-hfCas13f v2.5 system.

The toxicity of the AAV.PHP.eB particles was assessed at Week 7 after ICV administration using NeuN IF. The results showed the NeuN positive signal was similar in the whole brain of the administrated MDS mice to the negative control MDS mice without the ICV administration (FIG. 9), indicating that there was not observed toxicity at Week 7 after ICV administration.

In summary, the data presented herein demonstrated that the subject CRISPR-hfCas13f v2 and v2.5 systems efficiently reduced expression of MECP2 in cultured 293T cells and Cos7 cells in vitro and in mouse hip and whole brain in vivo, and the ICV administration of AAV.PHP.eB particles delivering the hfCas13f v2.5-dual-sgRNA[MECP2] system could recuse the abnormal behavior of MDS mice, showing the promising prospect of treating MDS and also the other MECP2 related diseases in human.

### Example 4. In Vivo Knockdown of Human MECP2 mRNA by CRISPR-Cas13 System Comprising hfCas13f.v v3

This Example demonstrates the in vivo knockdown efficiency of rAAV9 particles delivering the CRISPR-Cas13 system comprising hfCas13f.1 v3 (SEQ ID NO: 61) and sg9 & 3.

It was found in in vitro fluorescence reporter experiments that hfCas13f.1 v3 achieved significantly higher cleavage activity than hfCas13f.1 v2 and significantly lower collateral activity than hfCas13f.1 v2, and thus hfCas13f.1 v3 was used in replace of hfCas13f.1 v2 (FIG. 12).

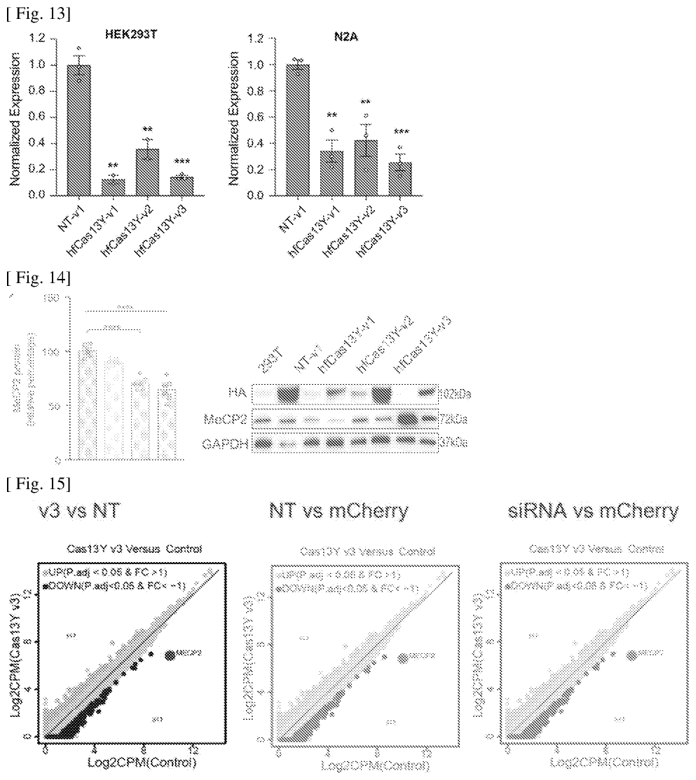

Furthermore, it was next examined the knockdown efficiency of MECP2 and the specificity of Cas13f-gMECP2, the plasmids containing different version of Cas13f.1 protein, together with sg9 and sg3 of MECP2, were transfected into the 293T cells. The qPCR and western blot results showed that among different versions of Cas13f.1, hfCas13f.1 v3 exhibited the highest knockdown efficiency of MECP2 (FIGS. 13 and 14). Whole transcriptome sequencing (RNA-seq) results showed that hfCas13f.1 v3 induced the lowest number of differentiated genes and most of these genes were MECP2-related genes, indicating the high specificity of hfCas13f.1 v3 targeting MECP2 with sg9 and sg3 (FIG. 15).

### Transgene Plasmid Construction:

A rAAV transgene plasmid for packaging into AAV9 capsid to prepare all-in-one rAAV9 particles was designed to comprise, from 5′ to 3′, the elements in Table 4 below. The transgene plasmid may encode one guide nucleic acid under the regulation of a promoter or two, three, or more guide nucleic acids under the regulation of a same promoter or separate promoters.

TABLE 4
Elements of the rAAV transgene plasmid

| Transgene plasmid element | SEQ ID NO: |
|---|---|
| AAV2 5′ ITR | 52 |
| U6 promoter | 49 |
| DR sequence | 6 |
| Spacer sequence 9 | 25 |
| DR sequence | 6 |
| Spacer sequence 3 | 19 |
| DR sequence | 6 |
| mMECP2 promoter | 63 |
| Kozak sequence | 54 |
| bpSV40 NLS coding sequence 1 | 64 |
| napRNAn coding sequence | 62 but without |
| | the first ATG |
| | codon |
| bpSV40 NLS coding sequence 2 | 65 |
| WPRE3 | 59 |
| sequence encoding bGH polyA signal | 48 |
| AAV2 3′ ITR | 53 |

The ITR-to-ITR sequence of the vector genome of the rAAV9 particle is set forth in SEQ ID NO: 66. The rAAV9 vector genome may also encode a HA tag for observation of distribution.

The rAAV9 particles were similarly prepared and administrated as described above except for the replacement of serotype AAV-PHP.eB with serotype AAV9. The mRNA and protein levels were similarly measured as described above.

### Results

The rAAV9 particles were administrated into the brain of P1 MDS mice by ICV injection. After 12 weeks, the expression of hfCas13f.1 v3 in the whole brain was detected by IF staining of HA. The hfCas13f.1 v3 was widely distributed across the brain. Then, the knockdown efficiency of MeCP2 in the cortex was tested by co-staining of HA and MeCP2, MECP2 TG mice, Mecp2^{+/y} mice and Mecp2^{-/y} mice as controls. The IF results showed that HA-tag was widely distributed in the cortex and the expression of MeCP2 was significantly decreased in HA-positive cells than it is in control MECP2 TG mice, and slightly higher than Mecp2^{+/y} mice. These results illustrate that AAV9-mediated hfCas13f.1 v3-gMECP2 editing effectively knocked down the MeCP2 in the whole brain of MECP2 TG mice.

To ensure the safety of MDS treatment and clinical application of AAV9-hfCas13f.1 v3-gMECP2, the HA tag in the construction was removed. In order to explore the optimal dose of rAAV9-hfCas13f.1 v3-gMECP2 in the humanized MECP2 TG mice, P1 MECP2 TG mice was treated with different dose (from 4.5E8vg/mice to 4.5E10vg/mice) of rAAV via ICV injection, followed by a series of tests to assess its efficacy in MECP2 TG mice. Four weeks after injection, the transduction efficiency of rAAV9-hfCas13f.1 v3-gMECP2 was detected in the whole brain by absolute quantitative PCR.

The results show that hfCas13f.1 v3 was widely expressed in the whole brain of the rAAV9-hfCas13f.1 v3-gMECP2 treated MECP2 TG mice (Figure S4B). Four weeks later, the expression of hfCas13f.1 v3, gRNA and MECP2 in the brain regions that are closely related to MDS such as cortex, striatum and hippocampus was analyzed by qPCR. Results show that the amounts of rAAV9-hfCas13f.1 v3-gMECP2 increased dose-dependently. Besides, the expression level of hfCas13f.1 v3 and gRNAs in hippocampus were less than they were in cortex and striatum. For the expression of MECP2, MeCP2 (representing mouse Mecp2 and MECP2 gene) and GFP (representing human MECP2 gene, GFP was fused at the C terminus of human MECP2 in MECP2 TG mice) were analyzed. Results showed that the expression of MeCP2 and GFP were decreased dose dependently in cortex, striatum and hippocampus after the treatment of rAAV9-hfCas13f.1 v3-gMECP2. For the cortex, the lowest effective dose which could effectively knock down the nRNA and protein of MECP2 in MECP2 TG mice was dose 2. In the striatum and hippocampus, the effective dose was dose 4. The highest dose (dose 5) normalized the expression of MECP2 in cortex and striatum. Besides, it was examined the expression of human MeCP2 protein with GFP antibody in the cortex of MECP2 TG mice that treated with the highest dose of rAAV9-hfCas13f.1 v3-gMECP2 by Western blot. The result show that the protein level of human MeCP2 was decreased by about 60% in rAAV9-hfCas13f.1 v3-gMECP2 treated group compared with MDS mice. Thus, the results indicate that human MECP2 can be effectively knocked down by rAAV9-hfCas13f.1 v3-gMECP2. Moreover, IF results showed that MeCP2 was normalized in cortex, striatum, CA1 and DG regions of the MECP2 TG mice after 12 weeks of the injection of rAAV9-hfCas13f.1 v3-gMECP2 via ICV.

To test whether normalizing MeCP2 expression can reverse the impaired behavioral phenotype of the MECP2 TG mice, behavioral tests was performed in the MECP2 TG mice after the administration of different doses of rAAV9-hfCas13f.1 v3-gMECP2 (dose 4 and dose 5) for 8 weeks. The accelerating rotarod test show that the prolonged latency to fall from the rotating rod decreased rAAV9-hfCas13f.1 v3-gMECP2 treated MECP2 TG mice at both doses. Besides, the open field test showed that the MECP2 TG mice displayed reduced exploratory behavior, which could be improved by the treatment of rAAV9-hfCas13f.1 v3-gMECP2 at the dose of dose 5. Moreover, the elevated plus maze test showed that the anxiety-like behavior of MECP2 TG mice was increased compared with Mecp2^{+/y} mice, and it could be greatly alleviated by the administration of rAAV9-hfCas13f.1 v3-gMECP2 at both doses. In the three-chamber social test, the MECP2 TG mice showed less communication behaviors, which could be greatly improved by the treatment of rAAV9-hfCas13f.1 v3-gMECP2 at both doses. Furthermore, it was also examined the expression of dysregulated genes in MDS such as Aw551984, Npbwr1, Crh, Efemp1, Oprk1 and Tsc22d3 (Carvalho et al., 2009; Samaco et al., 2012). The qPCR results revealed that the above genes are partially or totally normalized by the administration of rAAV9-hfCas13f.1 v3-gMECP2. In addition, it was performed RNA-seq on the cortex tissue four weeks after ICV injection of AAV9-hfCas13f.1 v3-gMECP2. The results showed abnormal gene expression in male MECP2 TG mice was largely rescued by hfCas13f.1 v3-gMECP2 treatment.

To test the treatment effect of rAAV9-hfCas13f.1 v3-gMECP2 in long term, it was performed behavioral tests in the MECP2 TG mice after the administration of rAAV9-hfCas13f.1 v3-gMECP2 for 26 weeks. The open field test showed that the MECP2 TG mice displayed reduced exploratory behavior, which could be improved by the treatment of rAAV9-hfCas13f.1 v3-gMECP2 at long term. Moreover, the elevated plus maze test showed that the anxiety-like behavior of MECP2 TG mice was increased compared with Mecp2^{+/y} mice, and it could be greatly alleviated by the administration of rAAV9-hfCas13f.1 v3-gMECP2 at long term. Interestingly, MECP2 TG mice injected with the AAV9-hfCas13f.1 v3-gMECP2 had a notable increase in median lifespan (156+−10.56 day) compared to littermates injected with the PBS. Five of six MECP2 TG mice treated with AAV9-hfCas13f.1 v3-gMECP2 survived over 220 days, whereas only one of ten MECP2 TG mice treated with PBS lived past 220 days. Therefore, a single administration of AAV9-hfCas13f.1 v3-gMECP2 into the central nervous system was sufficient to greatly prolong survival of MECP2 TG mice.

Studies showed that AAV might cause toxic effects and immunogenicity in long-term. Therefore, it was detected the concentrations of neurofilament light (NFL, a neuronal/axonal-injury biomarker), ALT and AST (liver-injury biomarker), BUN (kidney-injury biomarker), and interferon-γ (IFN-γ, an immunogenicity factor) in the serum of rAAV9-hfCas13f.1 v3-gMECP2 treated MECP2 TG mice after 12 weeks by ELISA analysis, and found no significant change. The above results revealed that there were barely neuronal/axonal injury or immunogenicity effects in rAAV9-hfCas13f.1 v3-gMECP2 treatment of MECP2 TG mice.

In addition, the P1 MECP2 TG mice was treated with 3.36E10 vg dose of rAAV9-hfCas13f.1 v3-gMECP2 via IT injection. It was found hfCas13f.1 v3 was mainly expressed in the spine. The distribution of hfCas13f.1 v3 in brain stem, cerebellum and brain was very limited and the expression of MECP2 did not change significantly in these regions. These results indicate that in comparison with IT injection, ICV injection of AAV9-hfCas13f.1 v3-gMECP2 might be better strategy for MDS treatment. 8-week-old male MECP2 TG mice was also treated with rAAV9-hfCas13f.1 v3-gMECP2 via ICV injection. It was found that hfCas13f.1 v3 and MECP2 sgRNAs were widely expressed in the cortex, striatum and hippocampus 4 weeks after injection, and the mRNA level of MECP2 in the rAAV9-hfCas13f.1 v3-gMECP2 treated MECP2 TG mice was significantly decreased compared with the control MECP2 TG mice, but was still higher than it was in Mecp2^{+/y} mice. The Western blot results showed that total protein level of MeCP2 in rAAV9-hfCas13f.1 v3-gMECP2 treated MECP2 TG mice was slight decreased compared to the MECP2 TG mice, and the human MeCP2 (GFP) protein in the rAAV9-hfCas13f.1 v3-gMECP2 treated MECP2 TG mice was significantly decreased compared with the MECP2 TG mice. Eight weeks after the administration of rAAV9-hfCas13f.1 v3-gMECP2, some abnormal behavioral phenotypes of the MECP2 TG mice, including prolonged latency to fall and exploratory behavior, were rescued by hfCas13f.1 v3-gMECP2 treatment. However, the impaired sociality behavior of 16-week-old MDS mice could not be rescued.

In summary, systemic administration of rAAV9-hfCas13f.1 v3-gMECP2 via ICV injection can efficiently knock down human MeCP2 in the cortex, striatum and hippocampus in P1 or adult male MECP2 TG mice. Moreover, it could partially normalize the expression of downstream genes in cortex. Importantly, the behavioral impairments of the MECP2 TG mice including motor, anxiety and sociality deficits could be significantly repaired by the administration of rAAV9-hfCas13f.1 v3-gMECP2 via ICV injection for 8-10 weeks.

Various modifications and variations of the described products, methods, and uses of the disclosure will be apparent to those skilled in the art without departing from the scope and spirit of the disclosure.

Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

EXEMPLARY SEQUENCES

Cas13f.1 amino acid sequence, SEQ ID NO: 1
MNGIELKKEEAAFYFNQAELNLKAIEDNIFDKERRKTLLNNPQILAKMENFIFNFRDVTKNAKGEIDCLLL
KLRELRNFYSHYVHKRDVRELSKGEKPILEKYYQFAIESTGSENVKLEIIENDAWLADAGVLFFLCIFLKK
SQANKLISGISGFKRNDDTGQPRRNLFTYFSIREGYKVVPEMQKHFLLFSLVNHLSNQDDYIEKAHQPYDI
GEGLFFHRIASTFLNISGILRNMKFYTYQSKRIVEQRGELKREKDIFAWEEPFQGNSYFEINGHKGVIGEDE
LKELCYAFLIGNQDANKVEGRITQFLEKFRNANSVQQVKDDEMLKPEYFPANYFAESGVGRIKDRVLNRL
NKAIKSNKAKKGEIIAYDKMREVMAFINNSLPVDEKLKPKDYKRYLGMVRFWDREKDNIKREFETKEWS
KYLPSNFWTAKNLERVYGLAREKNAELFNKLKADVEKMDERELEKYQKINDAKDLANLRRLASDFGVK
WEEKDWDEYSGQIKKQITDSQKLTIMKQRITAGLKKKHGIENLNLRITIDINKSRKAVLNRIAIPRGFVKRH
ILGWQESEKVSKKIREAECEILLSKEYEELSKQFFQSKDYDKMTRINGLYEKNKLIALMAVYLMGQLRILF
KEHTKLDDITKTTVDFKISDKVTVKIPFSNYPSLVYTMSSKYVDNIGNYGFSNKDKDKPILGKIDVIEKQR
MEFIKEVLGFEKYLFDDKIIDKSKFADTATHISFAEIVEELVEKGWDKDRLTKLKDARNKALHGEILTGTSF
DETKSLINELKK
hfCas13f.v2(Cas13f.1-Y666A + Y677A) amino acid sequence, SEQ ID NO: 2
MNGIELKKEEAAFYENQAELNLKAIEDNIFDKERRKTLLNNPQILAKMENFIFNFRDVTKNAKGEIDCLLL
KLRELRNFYSHYVHKRDVRELSKGEKPILEKYYQFAIESTGSENVKLEIIENDAWLADAGVLFFLCIFLKK
SQANKLISGISGFKRNDDTGQPRRNLFTYFSIREGYKVVPEMQKHFLLFSLVNHLSNQDDYIEKAHQPYDI
GEGLFFHRIASTFLNISGILRNMKFYTYQSKRIVEQRGELKREKDIFAWEEPFQGNSYFEINGHKGVIGEDE
LKELCYAFLIGNQDANKVEGRITQFLEKFRNANSVQQVKDDEMLKPEYFPANYFAESGVGRIKDRVLNRL
NKAIKSNKAKKGEIIAYDKMREVMAFINNSLPVDEKLKPKDYKRYLGMVRFWDREKDNIKREFETKEWS
KYLPSNFWTAKNLERVYGLAREKNAELFNKLKADVEKMDERELEKYQKINDAKDLANLRRLASDFGVK
WEEKDWDEYSGQIKKQITDSQKLTIMKQRITAGLKKKHGIENLNLRITIDINKSRKAVLNRIAIPRGFVKRH
ILGWQESEKVSKKIREAECEILLSKEYEELSKQFFQSKDYDKMTRINGLYEKNKLIALMAVYLMGQLRILF
KEHTKLDDITKTTVDFKISDKVTVKIPFSNAPSLVYTMSSKAVDNIGNYGFSNKDKDKPILGKIDVIEKQR
MEFIKEVLGFEKYLFDDKIIDKSKFADTATHISFAEIVEELVEKGWDKDRLTKLKDARNKALHGEILTGTSF
DETKSLINELKK
hfCas13f.v2(Cas13f.1-Y666A + Y677A) coding sequence, SEQ ID NO: 3
atgaatggcatcgagctgaagaaggaagaagccgccttctacttcaatcaggccgagctgaacctgaaggccattgaggacaacatct
tcgacaaggagagacggaagacactgctgaacaacccccagatcctggccaagatggagaactttatcttcaatttccgggacgtgaccaaga
acgccaagggcgaaatcgactgcctgctgctgaagctgagagagctgcggaacttttacagccactacgtgcacaagcgggacgtca
gagaactgagcaagggcgagaagccgatcctggagaagtactaccagttcgccatcgaatccaccggctctgagaacgtgaagctcgaaatc
atcgaaaacgacgcctggctggccgacgccggcgtgctgttcttcctgtgcatcttcctgaagaagagccaggcaaacaagctgatcagcggca
tcagcggcttcaagagaaacgacgacaccggccagcctcggagaaacctgttcacctacttctccatccgggagggctacaaggtggt
gcccgaaatgcagaagcacttcctgctgttctccctggtgaaccacctgagcaaccaggacgattatatcgaaaaggcccaccagccctacgac
atcggcgagggcctcttcttccaccggattgccagcaccttcctgaacatctccggaatcctgagaaacatgaagttctacacctatcaga
gcaagagactggtggagcagagaggcgagctgaagcgggaaaaggacatcttcgcctgggaagaaccgtttcagggcaattcctactttgag
atcaacggccacaagggcgtgattggcgaagacgagctgaaggagctgtgctacgccttcctgatcggcaaccaggacgccaacaaggtggaggg
ccggatcacccagttcctggagaagttcagaaacgccaacagcgtgcagcaggtgaaggacgacgagatgctgaagcctgaatatttccccgcca
actactttgccgagagcggcgtgggccggatcaaggaccgggtgctgaacagactgaacaaggccatcaagagcaacaaggccaagaagggcgaga
tcatcgcctatgacaagatgagagaagtgatggctttcatcaataactctctgcccgtggacgagaagctgaagcccaaggattacaagagata
cctgggcatggtgagattctgggatagagaaaaggacaatatcaagcgcgagttcgaaacgaaggagtggagcaagtatctgccctccaacttct
ggaccgccaagaacctggagagagtgtacggactggcccgggaaaagaacgcagagctgtttaacaagctgaaggccgacgtggagaagatggacga
aagagagctggaaaagtatcagaagatcaacgacgccaaggatctggccaacctgcggcggctggccagcgacttcggagtgaagtgggaggag
aaggattgggacgagtactccggccagatcaagaagcagatcacagattcccagaagctgaccatcatgaagcagagaatcacagccggcctgaag
aagaagcacggcatcgaaaacctgaacctgaggatcaccatcgacatcaacaagtccagaaaggccgtgctgaatcggatcgccatccccagagg
atttgtgaagcggcacatcctgggctggcaggaatccgagaaggtgagcaagaagatcagagaagccgaatgcgagattctgctgagcaaggagta
cgaggagctgagcaagcagttctttcagagcaaggactacgacaagatgacccgcatcaacggcctgtacgagaagaataagctgatcgccctg
atggccgtgtatctgatggggcagctgagaatcctgttcaaggagcacaccaagctggacgacatcaccaagaccaccgtggatttcaagatcagcg
acaaggtgaccgtgaagatccccttctccaacgctccctccctggtgtacaccatgagcagcaaggccgtggacaatatcggcaactacggctt
cagcaacaaggacaaggataagcccattctgggcaagatcgacgtgatcgagaagcagcggatggagtttatcaaggaggtgctgggattcgaga
agtacctgtttgacgataagatcatcgacaagagcaagttcgccgacaccgccacccacatcagctttgccgaaatcgtggaagaactggtggagaa
gggctgggacaaggaccggctgacgaagctgaaggatgcccggaacaaggccctgcacggcgagatcctgaccggcaccagcttcgacgagacaaagt
ccctgatcaacgagctgaagaag
hfCas13f.v2.5(Cas13f.1-L641A + Y666A + Y677A) amino acid sequence, SEQ ID NO: 4
MNGIELKKEEAAFYFNQAELNLKAIEDNIFDKERRKTLLNNPQILAKMENFIFNFRDVTKNAKGEIDCLLL
KLRELRNFYSHYVHKRDVRELSKGEKPILEKYYQFAIESTGSENVKLEIIENDAWLADAGVLFFLCIFLKK
SQANKLISGISGFKRNDDTGQPRRNLFTYFSIREGYKVVPEMQKHFLLFSLVNHLSNQDDYIEKAHQPYDI
GEGLFFHRIASTFLNISGILRNMKFYTYQSKRIVEQRGELKREKDIFAWEEPFQGNSYFEINGHKGVIGEDE
LKELCYAFLIGNQDANKVEGRITQFLEKFRNANSVQQVKDDEMLKPEYFPANYFAESGVGRIKDRVLNRL
NKAIKSNKAKKGEIIAYDKMREVMAFINNSLPVDEKLKPKDYKRYLGMVRFWDREKDNIKREFETKEWS
KYLPSNFWTAKNLERVYGLAREKNAELFNKLKADVEKMDERELEKYQKINDAKDLANLRRLASDFGVK
WEEKDWDEYSGQIKKQITDSQKLTIMKQRITAGLKKKHGIENLNLRITIDINKSRKAVLNRIAIPRGFVKRH
ILGWQESEKVSKKIREAECEILLSKEYEELSKQFFQSKDYDKMTRINGLYEKNKLIALMAVYLMGQLRILF
KEHTKADDITKTTVDFKISDKVTVKIPFSNAPSLVYTMSSKAVDNIGNYGFSNKDKDKPILGKIDVIEKQR
MEFIKEVLGFEKYLFDDKIIDKSKFADTATHISFAEIVEELVEKGWDKDRLTKLKDARNKALHGEILTGTSF
DETKSLINELKK
hfCas13f.v2.5(Cas13f.1-L641A + Y666A + Y677A) coding sequence, SEQ ID NO: 5
atgaatggcatcgagctgaagaaggaagaagccgccttctacttcaatcaggccgagctgaacctgaaggccattgaggacaacatcttcgacaa
ggagagacggaagacactgctgaacaacccccagatcctggccaagatggagaactttatcttcaatttccgggacgtgaccaagaacgccaaggg
cgaaatcgactgcctgctgctgaagctgagagagctgcggaacttttacagccactacgtgcacaagcgggacgtcagagaactgagcaagggcg
agaagccgatcctggagaagtactaccagttcgccatogaatccaccggctctgagaacgtgaagctcgaaatcatcgaaaacgacgcctggctgg
ccgacgccggcgtgctgttcttcctgtgcatcttcctgaagaagagccaggcaaacaagctgatcagcggcatcagcggcttcaagagaaacgacga
caccggccagcctcggagaaacctgttcacctacttctccatccgggagggctacaaggtggtgcccgaaatgcagaagcacttcctgctgttctcc
ctggtgaaccacctgagcaaccaggacgattatatcgaaaaggcccaccagccctacgacatcggcgagggcctcttcttccaccggattgccagcac
cttcctgaacatctccggaatcctgagaaacatgaagttctacacctatcagagcaagagactggtggagcagagaggcgagctgaagcgggaa
aaggacatcttcgcctgggaagaaccgtttcagggcaattcctactttgagatcaacggccacaagggcgtgattggcgaagacgagctgaagga
gctgtgctacgccttcctgatcggcaaccaggacgccaacaaggtggagggccggatcacccagttcctggagaagttcagaaacgccaac
agcgtgcagcaggtgaaggacgacgagatgctgaagcctgaatatttccccgccaactactttgccgagagcggcgtgggccggatcaaggaccgggt
gctgaacagactgaacaaggccatcaagagcaacaaggccaagaagggcgagatcatcgcctatgacaagatgagagaagtgatggctttcatca
ataactctctgcccgtggacgagaagctgaagcccaaggattacaagagatacctgggcatggtgagattctgggatagagaaaaggacaatatc
aagcgcgagttcgaaacgaaggagtggagcaagtatctgccctccaacttctggaccgccaagaacctggagagagtgtacggactggcc
cgggaaaagaacgcagagctgtttaacaagctgaaggccgacgtggagaagatggacgaaagagagctggaaaagtatcagaagatcaacg
acgccaaggatctggccaacctgcggcggctggccagcgacttcggagtgaagtgggaggagaaggattgggacgagtactccggccagatcaa
gaagcagatcacagattcccagaagctgaccatcatgaagcagagaatcacagccggcctgaagaagaagcacggcatcgaaaacctgaacctga
ggatcaccatcgacatcaacaagtccagaaaggccgtgctgaatoggatcgccatccccagaggatttgtgaagcggcacatcctgggctggcag
gaatccgagaaggtgagcaagaagatcagagaagccgaatgcgagattctgctgagcaaggagtacgaggagctgagcaagcagttctttcagagc
aaggactacgacaagatgacccgcatcaacggcctgtacgagaagaataagctgatcgccctgatggccgtgtatctgatggggcagctgagaatc
ctgttcaaggagcacaccaaggcggacgacatcaccaagaccaccgtggatttcaagatcagcgacaaggtgaccgtgaagatccccttctccaac
gccccctccctggtgtacaccatgagcagcaaggccgtggacaatatcggcaactacggcttcagcaacaaggacaaggataagcccatt
ctgggcaagatcgacgtgatcgagaagcagcggatggagtttatcaaggaggtgctgggattcgagaagtacctgtttgacgataagatcatcg
acaagagcaagttcgccgacaccgccacccacatcagctttgccgaaatcgtggaagaactggtggagaagggctgggacaaggaccggctgacga
agctgaaggatgcccggaacaaggccctgcacggcgagatcctgaccggcaccagcttcgacgagacaaagtccctgatcaacgagctgaagaag
DR Sequence, SEQ ID NO: 6
GCTGTGATAGACCTCGATTTGTGGGGTAGTAACAGC
gRNA1, SEQ ID NO: 7
GCTGTGATAGACCTCGATTTGTGGGGTAGTAACAGCCTGCGCCCCTTGGGGCTGCTCTCCTTGCTT
GCTGTGATAGACCTCGATTTGTGGGGTAGTAACAGC
gRNA2, SEQ ID NO: 8
GCTGTGATAGACCTCGATTTGTGGGGTAGTAACAGCGAAGACTCCTTCACGGCTTTCTTTTTGGCC
GCTGTGATAGACCTCGATTTGTGGGGTAGTAACAGC
gRNA3, SEQ ID NO: 9
GCTGTGATAGACCTCGATTTGTGGGGTAGTAACAGCGGGCTTCACCACTTCCTTGACCTCGATGCT
GCTGTGATAGACCTCGATTTGTGGGGTAGTAACAGC
gRNA4, SEQ ID NO: 10
GCTGTGATAGACCTCGATTTGTGGGGTAGTAACAGCAAAGTCAGCTAACTCTCTCGGTCACGGGC
GGCTGTGATAGACCTCGATTTGTGGGGTAGTAACAGC
gRNA5, SEQ ID NO: 11
GCTGTGATAGACCTCGATTTGTGGGGTAGTAACAGCTTGCGCTCTCCCTCCCCTCGGTGTTTGTAC
GCTGTGATAGACCTCGATTTGTGGGGTAGTAACAGC
gRNA6, SEQ ID NO: 12
GCTGTGATAGACCTCGATTTGTGGGGTAGTAACAGCGTCCACAGGCTCCTCTCTGTTTGGCCTTGG
GCTGTGATAGACCTCGATTTGTGGGGTAGTAACAGC
gRNA7, SEQ ID NO: 13
GCTGTGATAGACCTCGATTTGTGGGGTAGTAACAGCGATTTGGGCTTCTTAGGTGGTTTCTGCTCT
GCTGTGATAGACCTCGATTTGTGGGGTAGTAACAGC
gRNA8, SEQ ID NO: 14
GCTGTGATAGACCTCGATTTGTGGGGTAGTAACAGCGGCTGAGTCTTAGCTGGCTCCTTGGGGCA
GGCTGTGATAGACCTCGATTTGTGGGGTAGTAACAGC
gRNA9, SEQ ID NO: 15
GCTGTGATAGACCTCGATTTGTGGGGTAGTAACAGCGTTACCGTGAAGTCAAAATCATTAGGGTCC
GCTGTGATAGACCTCGATTTGTGGGGTAGTAACAGC
gRNA10, SEQ ID NO: 16
GCTGTGATAGACCTCGATTTGTGGGGTAGTAACAGCGTTACCGTGAAGTCAAAATCATTAGGGTCC
GCTGTGATAGACCTCGATTTGTGGGGTAGTAACAGC
Spacer1 Sequence, SEQ ID NO: 17
CTGCGCCCCTTGGGGCTGCTCTCCTTGCTT
Spacer2 Sequence, SEQ ID NO: 18
GAAGACTCCTTCACGGCTTTCTTTTTGGCC
Spacer3 Sequence, SEQ ID NO: 19
GGGCTTCACCACTTCCTTGACCTCGATGCT
Spacer4 Sequence, SEQ ID NO: 20
AAAGTCAGCTAACTCTCTCGGTCACGGGCG
Spacer5 Sequence, SEQ ID NO: 21
TTGCGCTCTCCCTCCCCTCGGTGTTTGTAC
Spacer6 Sequence, SEQ ID NO: 22
GTCCACAGGCTCCTCTCTGTTTGGCCTTGG
Spacer7 Sequence, SEQ ID NO: 23
GATTTGGGCTTCTTAGGTGGTTTCTGCTCT
Spacer8 Sequence, SEQ ID NO: 24
GGCTGAGTCTTAGCTGGCTCCTTGGGGCAG
Spacer9 Sequence, SEQ ID NO: 25
GTTACCGTGAAGTCAAAATCATTAGGGTCC
Spacer10 Sequence, SEQ ID NO: 26
GTTACCGTGAAGTCAAAATCATTAGGGTCC
Target Sequence 1, SEQ ID NO: 27
AAGCAAGGAGAGCAGCCCCAAGGGGCGCAG
Target Sequence 2, SEQ ID NO: 28
GGCCAAAAAGAAAGCCGTGAAGGAGTCTTC
Target Sequence 3, SEQ ID NO: 29
AGCATCGAGGTCAAGGAAGTGGTGAAGCCC
Target Sequence 4, SEQ ID NO: 30
GCCCGTGACCGAGAGAGTTAGCTGACTTT
Target Sequence 5, SEQ ID NO: 31
GTACAAACACCGAGGGGAGGGAGAGCGCAA
Target Sequence 6, SEQ ID NO: 32
CCAAGGCCAAACAGAGAGGAGCCTGTGGAC
Target Sequence 7, SEQ ID NO: 33
AGAGCAGAAACCACCTAAGAAGCCCAAATC
Target Sequence 8, SEQ ID NO: 34
CTGCCCCAAGGAGCCAGCTAAGACTCAGCC
Target Sequence 9, SEQ ID NO: 35
GGACCCTAATGATTTTGACTTCACGGTAAC
Target Sequence 10, SEQ ID NO: 36
ATCAAGAAGCGCAAGACCCGGGAGACGGTC
gRNA9&3, SEQ ID NO: 37
GCTGTGATAGACCTCGATTTGTGGGGTAGTAACAGCGTTACCGTGAAGTCAAAATCATTAGGGTCC
GCTGTGATAGACCTCGATTTGTGGGGTAGTAACAGCGGGCTTCACCACTTCCTTGACCTCGATGCT
GCTGTGATAGACCTCGATTTGTGGGGTAGTAACAGC
gRNA9&4, SEQ ID NO: 38
GCTGTGATAGACCTCGATTTGTGGGGTAGTAACAGCGTTACCGTGAAGTCAAAATCATTAGGGTCC
GCTGTGATAGACCTCGATTTGTGGGGTAGTAACAGCAAAGTCAGCTAACTCTCTCGGTCACGGGC
GGCTGTGATAGACCTCGATTTGTGGGGTAGTAACAGC
gRNA9&5, SEQ ID NO: 39
GCTGTGATAGACCTCGATTTGTGGGGTAGTAACAGCGTTACCGTGAAGTCAAAATCATTAGGGTCC
GCTGTGATAGACCTCGATTTGTGGGGTAGTAACAGCTTGCGCTCTCCCTCCCCTCGGTGTTTGTAC
GCTGTGATAGACCTCGATTTGTGGGGTAGTAACAGC
gRNA9&6, SEQ ID NO: 40
GCTGTGATAGACCTCGATTTGTGGGGTAGTAACAGCGTTACCGTGAAGTCAAAATCATTAGGGTCC
GCTGTGATAGACCTCGATTTGTGGGGTAGTAACAGC
GTCCACAGGCTCCTCTCTGTTTGGCCTTGGGCTGTGATAGACCTCGATTTGTGGGGTAGTAACAGC
Non-targeting gRNA, SEQ ID NO: 41
GCTGTGATAGACCTCGATTTGTGGGGTAGTAACAGCGAGACCCAGATCTGCCGGTCTCTGCTGTG
ATAGACCTCGATTTGTGGGGTAGTAACAGC
Non-targeting spacer sequence, SEQ ID NO: 42
GAGACCCAGATCTGCCGGTCTCT
Non-targeting target sequence, SEQ ID NO: 43
AGAGACCGGCAGATCTGGGTCTC
Cbh promoter, SEQ ID NO: 44
CGTTACATAACTTACGGTAAATGGCCCGCCTGGCTGACCGCCCAACGACCCCCGCCCATTGACGTCAA
TAGTAACGCCAATAGGGACTTTCCATTGACGTCAATGGGTGGAGTATTTACGGTAAACTGCCCACTTG
GCAGTACATCAAGTGTATCATATGCCAAGTACGCCCCCTATTGACGTCAATGACGGTAAATGGCCCGCC
TGGCATTGTGCCCAGTACATGACCTTATGGGACTTTCCTACTTGGCAGTACATCTACGTATTAGTCATCG
CTATTACCATGGTCGAGGTGAGCCCCACGTTCTGCTTCACTCTCCCCATCTCCCCCCCCTCCCCACCCC
CAATTTTGTATTTATTTATTTTTTAATTATTTTGTGCAGCGATGGGGGCGGGGGGGGGGGGGGGGCGCG
CGCCAGGCGGGGCGGGGCGGGGCGAGGGGCGGGGCGGGGCGAGGCGGAGAGGTGCGGCGGCAGC
CAATCAGAGCGGCGCGCTCCGAAAGTTTCCTTTTATGGCGAGGCGGCGGCGGCGGCGGCCCTATAAA
AAGCGAAGCGCGCGGCGGGGGGAGTCGCTGCGCGCTGCCTTCGCCCCGTGCCCCGCTCCGCCGCC
GCCTCGCGCCGCCCGCCCCGGCTCTGACTGACCGCGTTACTCCCACAGGTGAGCGGGCGGGACGGCC
CTTCTCCTCCGGGCTGTAATTAGCTGAGCAAGAGGTAAGGGTTTAAGGGATGGTTGGTTGGTGGGGTA
TTAATGTTTAATTACCTGGAGCACCTGCCTGAAATCACTTTTTTTCAGGTTGG
Enhancer, SEQ ID NO: 45
CGTTACATAACTTACGGTAAATGGCCCGCCTGGCTGACCGCCCAACGACCCCCGCCCATTGACGTCAA
TAGTAACGCCAATAGGGACTTTCCATTGACGTCAATGGGTGGAGTATTTACGGTAAACTGCCCACTTG
GCAGTACATCAAGTGTATCATATGCCAAGTACGCCCCCTATTGACGTCAATGACGGTAAATGGCCCGCC
TGGCATTGTGCCCAGTACATGACCTTATGGGACTTTCCTACTTGGCAGTACATCTACGTATTAGTCATCG
CTATTACCATG
Intron, SEQ ID NO: 46
GGAGTCGCTGCGCGCTGCCTTCGCCCCGTGCCCCGCTCCGCCGCCGCCTCGCGCCGCCCGCCCCGGC
TCTGACTGACCGCGTTACTCCCACAGGTGAGCGGGCGGGACGGCCCTTCTCCTCCGGGCTGTAATTAG
CTGAGCAAGAGGTAAGGGTTTAAGGGATGGTTGGTTGGTGGGGTATTAATGTTTAATTACCTGGAGCA
CCTGCCTGAAATCACTTTTTTTCAG
SV40 NLS amino acid sequence, SEQ ID NO: 47
PKKKRKV
Sequence encoding bGH polyA signal, SEQ ID NO: 48
CTGTGCCTTCTAGTTGCCAGCCATCTGTTGTTTGCCCCTCCCCCGTGCCTTCCTTGACCCTGGAAGGTG
CCACTCCCACTGTCCTTTCCTAATAAAATGAGGAAATTGCATCGCATTGTCTGAGTAGGTGTCATTCTA
TTCTGGGGGGTGGGGTGGGGCAGGACAGCAAGGGGGAGGATTGGGAAGACAATAGCAGGCATGCTG
GGGATGCGGTGGGCTCTATGG
U6 promoter, SEQ ID NO: 49
GAGGGCCTATTTCCCATGATTCCTTCATATTTGCATATACGATACAAGGCTGTTAGAGAGATAATTGGAA
TTAATTTGACTGTAAACACAAAGATATTAGTACAAAATACGTGACGTAGAAAGTAATAATTTCTTGGGT
AGTTTGCAGTTTTAAAATTATGTTTTAAAATGGACTATCATATGCTTACCGTAACTTGAAAGTATTTCGA
TTTCTTGGCTTTATATATCTTGTGGAAAGGACGAAACACCG
CMV promoter, SEQ ID NO: 50
GTGATGCGGTTTTGGCAGTACATCAATGGGCGTGGATAGCGGTTTGACTCACGGGGATTTCCAAGTCT
CCACCCCATTGACGTCAATGGGAGTTTGTTTTGGCACCAAAATCAACGGGACTTTCCAAAATGTCGTA
ACAACTCCGCCCCATTGACGCAAATGGGCGGTAGGCGTGTACGGTGGGAGGTCTATATAAGCAGAGCT
mCherry amino acid sequence, SEQ ID NO: 51
MVSKGEEDNMAIIKEFMRFKVHMEGSVNGHEFEIEGEGEGRPYEGTQTAKLKVTKGGPLPFAWDILSPQF
MYGSKAYVKHPADIPDYLKLSFPEGFKWERVMNFEDGGVVTVTQDSSLQDGEFIYKVKLRGTNFPSDGP
VMQKKTMGWEASSERMYPEDGALKGEIKQRLKLKDGGHYDAEVKTTYKAKKPVQLPGAYNVNIKLDI
TSHNEDYTIVEQYERAEGRHSTGGMDELYK
AAV2 5' ITR, SEQ ID NO: 52
CTGCGCGCTCGCTCGCTCACTGAGGCCGCCCGGGCAAAGCCCGGGCGTCGGGCGACCTTTGGTCGCC
CGGCCTCAGTGAGCGAGCGAGCGCGCAGAGAGGGAGTGGCCAACTCCATCACTAGGGGTTCCT
AAV2 3' ITR, SEQ ID NO: 53
AGGAACCCCTAGTGATGGAGTTGGCCACTCCCTCTCTGCGCGCTCGCTCGCTCACTGAGGCCGGGCG
ACCAAAGGTCGCCCGACGCCCGGGCTTTGCCCGGGCGGCCTCAGTGAGCGAGCGAGCGCGCAG
Kozak sequence, SEQ ID NO: 54
gccacc
HA tag, SEQ ID NO: 55
YPYDVPDYA
AAV-PHP.eB transgene plasmid- 8 hfCas13f.v2 9 -gRNA 9&3 ITR-to-ITR, SEQ ID NO: 56
CTGCGCGCTCGCTCGCTCACTGAGGCCGCCCGGGCAAAGCCCGGGCGTCGGGCGACCTTTGGTCGCC
CGGCCTCAGTGAGCGAGCGAGCGCGCAGAGAGGGAGTGGCCAACTCCATCACTAGGGGTTCCTGCG
GCCATCGATGGCGCGCCCGTTACATAACTTACGGTAAATGGCCCGCCTGGCTGACCGCCCAACGACCC
CCGCCCATTGACGTCAATAGTAACGCCAATAGGGACTTTCCATTGACGTCAATGGGTGGAGTATTTACG
GTAAACTGCCCACTTGGCAGTACATCAAGTGTATCATATGCCAAGTACGCCCCCTATTGACGTCAATGA
CGGTAAATGGCCCGCCTGGCATTGTGCCCAGTACATGACCTTATGGGACTTTCCTACTTGGCAGTACAT
CTACGTATTAGTCATCGCTATTACCATGGTCGAGGTGAGCCCCACGTTCTGCTTCACTCTCCCCATCTCC
CCCCCCTCCCCACCCCCAATTTTGTATTTATTTATTTTTTAATTATTTTGTGCAGCGATGGGGGCGGGGG
GGGGGGGGGGGGCGCGCCAGGCGGGGCGGGGCGGGGCGAGGGGCGGGGCGGGGCGAGGCGGAG
AGGTGCGGCGGCAGCCAATCAGAGCGGCGCGCTCCGAAAGTTTCCTTTTATGGCGAGGCGGCGGCGG
CGGCGGCCCTATAAAAAGCGAAGCGCGCGGCGGGCGGGAGTCGCTGCGCGCTGCCTTCGCCCCGTG
CCCCGCTCCGCCGCCGCCTCGCGCCGCCCGCCCCGGCTCTGACTGACCGCGTTACTCCCACAGGTGA
GCGGGCGGGACGGCCCTTCTCCTCCGGGCTGTAATTAGCTGAGCAAGAGGTAAGGGTTTAAGGGATG
GTTGGTTGGTGGGGTATTAATGTTTAATTACCTGGAGCACCTGCCTGAAATCACTTTTTTTCAGGTTGG
ACCGGTGCCACCATGCCCAAGAAGAAGCGGAAGGTGAATGGCATCGAGCTGAAGAAGGAAGAAGCC
GCCTTCTACTTCAATCAGGCCGAGCTGAACCTGAAGGCCATTGAGGACAACATCTTCGACAAGGAGA
GACGGAAGACACTGCTGAACAACCCCCAGATCCTGGCCAAGATGGAGAACTTTATCTTCAATTTCCG
GGACGTGACCAAGAACGCCAAGGGCGAAATCGACTGCCTGCTGCTGAAGCTGAGAGAGCTGCGGAA
CTTTTACAGCCACTACGTGCACAAGCGGGACGTCAGAGAACTGAGCAAGGGCGAGAAGCCGATCCT
GGAGAAGTACTACCAGTTCGCCATCGAATCCACCGGCTCTGAGAACGTGAAGCTCGAAATCATCGAA
AACGACGCCTGGCTGGCCGACGCCGGCGTGCTGTTCTTCCTGTGCATCTTCCTGAAGAAGAGCCAGG
CAAACAAGCTGATCAGCGGCATCAGCGGCTTCAAGAGAAACGACGACACCGGCCAGCCTCGGAGAA
ACCTGTTCACCTACTTCTCCATCCGGGAGGGCTACAAGGTGGTGCCCGAAATGCAGAAGCACTTCCT
GCTGTTCTCCCTGGTGAACCACCTGAGCAACCAGGACGATTATATCGAAAAGGCCCACCAGCCCTAC
GACATCGGCGAGGGCCTCTTCTTCCACCGGATTGCCAGCACCTTCCTGAACATCTCCGGAATCCTGAG
AAACATGAAGTTCTACACCTATCAGAGCAAGAGACTGGTGGAGCAGAGAGGCGAGCTGAAGCGGGA
AAAGGACATCTTCGCCTGGGAAGAACCGTTTCAGGGCAATTCCTACTTTGAGATCAACGGCCACAAG
GGCGTGATTGGCGAAGACGAGCTGAAGGAGCTGTGCTACGCCTTCCTGATCGGCAACCAGGACGCCA
ACAAGGTGGAGGGCCGGATCACCCAGTTCCTGGAGAAGTTCAGAAACGCCAACAGCGTGCAGCAGG
TGAAGGACGACGAGATGCTGAAGCCTGAATATTTCCCCGCCAACTACTTTGCCGAGAGCGGCGTGGG
CCGGATCAAGGACCGGGTGCTGAACAGACTGAACAAGGCCATCAAGAGCAACAAGGCCAAGAAGG
GCGAGATCATCGCCTATGACAAGATGAGAGAAGTGATGGCTTTCATCAATAACTCTCTGCCCGTGGAC
GAGAAGCTGAAGCCCAAGGATTACAAGAGATACCTGGGCATGGTGAGATTCTGGGATAGAGAAAAGG
ACAATATCAAGCGCGAGTTCGAAACGAAGGAGTGGAGCAAGTATCTGCCCTCCAACTTCTGGACCGC
CAAGAACCTGGAGAGAGTGTACGGACTGGCCCGGGAAAAGAACGCAGAGCTGTTTAACAAGCTGAA
GGCCGACGTGGAGAAGATGGACGAAAGAGAGCTGGAAAAGTATCAGAAGATCAACGACGCCAAGGA
TCTGGCCAACCTGCGGCGGCTGGCCAGCGACTTCGGAGTGAAGTGGGAGGAGAAGGATTGGGACGA
GTACTCCGGCCAGATCAAGAAGCAGATCACAGATTCCCAGAAGCTGACCATCATGAAGCAGAGAATC
ACAGCCGGCCTGAAGAAGAAGCACGGCATCGAAAACCTGAACCTGAGGATCACCATCGACATCAAC
AAGTCCAGAAAGGCCGTGCTGAATCGGATCGCCATCCCCAGAGGATTTGTGAAGCGGCACATCCTGG
GCTGGCAGGAATCCGAGAAGGTGAGCAAGAAGATCAGAGAAGCCGAATGCGAGATTCTGCTGAGCA
AGGAGTACGAGGAGCTGAGCAAGCAGTTCTTTCAGAGCAAGGACTACGACAAGATGACCCGCATCA
ACGGCCTGTACGAGAAGAATAAGCTGATCGCCCTGATGGCCGTGTATCTGATGGGGCAGCTGAGAATC
CTGTTCAAGGAGCACACCAAGCTGGACGACATCACCAAGACCACCGTGGATTTCAAGATCAGCGACA
AGGTGACCGTGAAGATCCCCTTCTCCAACGCTCCCTCCCTGGTGTACACCATGAGCAGCAAGGCCGT
GGACAATATCGGCAACTACGGCTTCAGCAACAAGGACAAGGATAAGCCCATTCTGGGCAAGATCGAC
GTGATCGAGAAGCAGCGGATGGAGTTTATCAAGGAGGTGCTGGGATTCGAGAAGTACCTGTTTGACG
ATAAGATCATCGACAAGAGCAAGTTCGCCGACACCGCCACCCACATCAGCTTTGCCGAAATCGTGGA
AGAACTGGTGGAGAAGGGCTGGGACAAGGACCGGCTGACGAAGCTGAAGGATGCCCGGAACAAGG
CCCTGCACGGCGAGATCCTGACCGGCACCAGCTTCGACGAGACAAAGTCCCTGATCAACGAGCTGA
AGAAGCCCAAGAAGAAGCGGAAGGTGTACCCATACGATGTTCCAGATTACGCTTATCCCTACGACGTG
CCTGATTATGCATACCCATATGATGTCCCCGACTATGCCTAGAAGCTTCTGTGCCTTCTAGTTGCCAGCC
ATCTGTTGTTTGCCCCTCCCCCGTGCCTTCCTTGACCCTGGAAGGTGCCACTCCCACTGTCCTTTCCTA
ATAAAATGAGGAAATTGCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGATGCGGTGGGCTCTATGG
CTTACTAGTGAGGGCCTATTTCCCATGATTCCTTCATATTTGCATATACGATACAAGGCTGTTAGAGAGA
TAATTGGAATTAATTTGACTGTAAACACAAAGATATTAGTACAAAATACGTGACGTAGAAAGTAATAAT
TTCTTGGGTAGTTTGCAGTTTTAAAATTATGTTTTAAAATGGACTATCATATGCTTACCGTAACTTGAAA
GTATTTCGATTTCTTGGCTTTATATATCTTGTGGAAAGGACGAAACACCGGCTGTGATAGACCTCGAT
TTGTGGGGTAGTAACAGCGTTACCGTGAAGTCAAAATCATTAGGGTCCGCTGTGATAGACCTCGAT
TTGTGGGGTAGTAACAGCGGGCTTCACCACTTCCTTGACCTCGATGCTGCTGTGATAGACCTCGAT
TTGTGGGGTAGTAACAGCTTTTTTTGAATCCATCGGCGGCCGCGTCGGCCGCAGGAACCCCTAGTGA
TGGAGTTGGCCACTCCCTCTCTGCGCGCTCGCTCGCTCACTGAGGCCGGGCGACCAAAGGTCGCCCG
ACGCCCGGGCTTTGCCCGGGCGGCCTCAGTGAGCGAGCGAGCGCGCAG
AAV-PHP.eB transgene plasmid- 8 hfCas13f.v2.5 9 -gRNA 9&3 ITR-to-ITR, SEQ ID NO: 57
CTGCGCGCTCGCTCGCTCACTGAGGCCGCCCGGGCAAAGCCCGGGCGTCGGGCGACCTTTGGTCGCC
CGGCCTCAGTGAGCGAGCGAGCGCGCAGAGAGGGAGTGGCCAACTCCATCACTAGGGGTTCCTGCG
GCCATCGATGGACGCGTGAGGGCCTATTTCCCATGATTCCTTCATATTTGCATATACGATACAAGGCTGT
TAGAGAGATAATTGGAATTAATTTGACTGTAAACACAAAGATATTAGTACAAAATACGTGACGTAGAA
AGTAATAATTTCTTGGGTAGTTTGCAGTTTTAAAATTATGTTTTAAAATGGACTATCATATGCTTACCGTA
ACTTGAAAGTATTTCGATTTCTTGGCTTTATATATCTTGTGGAAAGGACGAAACACCGGCTGTGATAG
ACCTCGATTTGTGGGGTAGTAACAGCGTTACCGTGAAGTCAAAATCATTAGGGTCCGCTGTGATAG
ACCTCGATTTGTGGGGTAGTAACAGCGGGCTTCACCACTTCCTTGACCTCGATGCTGCTGTGATAG
ACCTCGATTTGTGGGGTAGTAACAGCTTTTTTTCCCGTTACATAACTTACGGTAAATGGCCCGCCTG
GCTGACCGCCCAACGACCCCCGCCCATTGACGTCAATAGTAACGCCAATAGGGACTTTCCATTGACGT
CAATGGGTGGAGTATTTACGGTAAACTGCCCACTTGGCAGTACATCAAGTGTATCATATGCCAAGTACG
CCCCCTATTGACGTCAATGACGGTAAATGGCCCGCCTGGCATTGTGCCCAGTACATGACCTTATGGGA
CTTTCCTACTTGGCAGTACATCTACGTATTAGTCATCGCTATTACCATGGTCGAGGTGAGCCCCACGTTC
TGCTTCACTCTCCCCATCTCCCCCCCCTCCCCACCCCCAATTTTGTATTTATTTATTTTTTAATTATTTTGT
GCAGCGATGGGGGCGGGGGGGGGGGGGGGGCGCGCGCCAGGCGGGGCGGGGCGGGGCGAGGGGC
GGGGCGGGGCGAGGCGGAGAGGTGCGGCGGCAGCCAATCAGAGCGGCGCGCTCCGAAAGTTTCCTT
TTATGGCGAGGCGGCGGCGGCGGCGGCCCTATAAAAAGCGAAGCGCGCGGCGGGCGGGAGTCGCTG
CGCGCTGCCTTCGCCCCGTGCCCCGCTCCGCCGCCGCCTCGCGCCGCCCGCCCCGGCTCTGACTGAC
CGCGTTACTCCCACAGGTGAGCGGGCGGGACGGCCCTTCTCCTCCGGGCTGTAATTAGCTGAGCAAG
AGGTAAGGGTTTAAGGGATGGTTGGTTGGTGGGGTATTAATGTTTAATTACCTGGAGCACCTGCCTGA
AATCACTTTTTTTCAGGTTGGACCGGTGCCACCATGAAACGGACAGCCGACGGAAGCGAGTTCGAGT
CACCCAAGAAGAAGCGGAAGGTGAATGGCATCGAGCTGAAGAAGGAAGAAGCCGCCTTCTACTTCA
ATCAGGCCGAGCTGAACCTGAAGGCCATTGAGGACAACATCTTCGACAAGGAGAGACGGAAGACAC
TGCTGAACAACCCCCAGATCCTGGCCAAGATGGAGAACTTTATCTTCAATTTCCGGGACGTGACCAAG
AACGCCAAGGGCGAAATCGACTGCCTGCTGCTGAAGCTGAGAGAGCTGCGGAACTTTTACAGCCACT
ACGTGCACAAGCGGGACGTCAGAGAACTGAGCAAGGGCGAGAAGCCGATCCTGGAGAAGTACTACC
AGTTCGCCATCGAATCCACCGGCTCTGAGAACGTGAAGCTCGAAATCATCGAAAACGACGCCTGGCT
GGCCGACGCCGGCGTGCTGTTCTTCCTGTGCATCTTCCTGAAGAAGAGCCAGGCAAACAAGCTGATC
AGCGGCATCAGCGGCTTCAAGAGAAACGACGACACCGGCCAGCCTCGGAGAAACCTGTTCACCTAC
TTCTCCATCCGGGAGGGCTACAAGGTGGTGCCCGAAATGCAGAAGCACTTCCTGCTGTTCTCCCTGGT
GAACCACCTGAGCAACCAGGACGATTATATCGAAAAGGCCCACCAGCCCTACGACATCGGCGAGGGC
CTCTTCTTCCACCGGATTGCCAGCACCTTCCTGAACATCTCCGGAATCCTGAGAAACATGAAGTTCTA
CACCTATCAGAGCAAGAGACTGGTGGAGCAGAGAGGCGAGCTGAAGCGGGAAAAGGACATCTTCGC
CTGGGAAGAACCGTTTCAGGGCAATTCCTACTTTGAGATCAACGGCCACAAGGGCGTGATTGGCGAA
GACGAGCTGAAGGAGCTGTGCTACGCCTTCCTGATCGGCAACCAGGACGCCAACAAGGTGGAGGGC
CGGATCACCCAGTTCCTGGAGAAGTTCAGAAACGCCAACAGCGTGCAGCAGGTGAAGGACGACGAG
ATGCTGAAGCCTGAATATTTCCCCGCCAACTACTTTGCCGAGAGCGGCGTGGGCCGGATCAAGGACC
GGGTGCTGAACAGACTGAACAAGGCCATCAAGAGCAACAAGGCCAAGAAGGGCGAGATCATCGCCT
ATGACAAGATGAGAGAAGTGATGGCTTTCATCAATAACTCTCTGCCCGTGGACGAGAAGCTGAAGCC
CAAGGATTACAAGAGATACCTGGGCATGGTGAGATTCTGGGATAGAGAAAAGGACAATATCAAGCGC
GAGTTCGAAACGAAGGAGTGGAGCAAGTATCTGCCCTCCAACTTCTGGACCGCCAAGAACCTGGAG
AGAGTGTACGGACTGGCCCGGGAAAAGAACGCAGAGCTGTTTAACAAGCTGAAGGCCGACGTGGAG
AAGATGGACGAAAGAGAGCTGGAAAAGTATCAGAAGATCAACGACGCCAAGGATCTGGCCAACCTG
CGGCGGCTGGCCAGCGACTTCGGAGTGAAGTGGGAGGAGAAGGATTGGGACGAGTACTCCGGCCAG
ATCAAGAAGCAGATCACAGATTCCCAGAAGCTGACCATCATGAAGCAGAGAATCACAGCCGGCCTGA
AGAAGAAGCACGGCATCGAAAACCTGAACCTGAGGATCACCATCGACATCAACAAGTCCAGAAAGG
CCGTGCTGAATCGGATCGCCATCCCCAGAGGATTTGTGAAGCGGCACATCCTGGGCTGGCAGGAATC
CGAGAAGGTGAGCAAGAAGATCAGAGAAGCCGAATGCGAGATTCTGCTGAGCAAGGAGTACGAGGA
GCTGAGCAAGCAGTTCTTTCAGAGCAAGGACTACGACAAGATGACCCGCATCAACGGCCTGTACGAG
AAGAATAAGCTGATCGCCCTGATGGCCGTGTATCTGATGGGGCAGCTGAGAATCCTGTTCAAGGAGCA
CACCAAGGCGGACGACATCACCAAGACCACCGTGGATTTCAAGATCAGCGACAAGGTGACCGTGAA
GATCCCCTTCTCCAACGCCCCCTCCCTGGTGTACACCATGAGCAGCAAGGCCGTGGACAATATCGGCA
ACTACGGCTTCAGCAACAAGGACAAGGATAAGCCCATTCTGGGCAAGATCGACGTGATCGAGAAGCA
GCGGATGGAGTTTATCAAGGAGGTGCTGGGATTCGAGAAGTACCTGTTTGACGATAAGATCATCGACA
AGAGCAAGTTCGCCGACACCGCCACCCACATCAGCTTTGCCGAAATCGTGGAAGAACTGGTGGAGA
AGGGCTGGGACAAGGACCGGCTGACGAAGCTGAAGGATGCCCGGAACAAGGCCCTGCACGGCGAG
ATCCTGACCGGCACCAGCTTCGACGAGACAAAGTCCCTGATCAACGAGCTGAAGAAGTCTGGCGGCT
CAAAAAGAACCGCCGACGGCAGCGAATTCGAGTCACCCAAGAAGAAGCGGAAGGTGGGATCCTACC
CATACGATGTTCCAGATTACGCTTATCCCTACGACGTGCCTGATTATGCATACCCATATGATGTCCCCGA
CTATGCCTAGAAGCTTAATCAACCTCTGGATTACAAAATTTGTGAAAGATTGACTGGTATTCTTAACTAT
GTTGCTCCTTTTACGCTATGTGGATACGCTGCTTTAATGCCTTTGTATCATGCTATTGCTTCCCGTATGGC
TTTCATTTTCTCCTCCTTGTATAAATCCTGGTTAGTTCTTGCCACGGCGGAACTCATCGCCGCCTGCCTT
GCCCGCTGCTGGACAGGGGCTCGGCTGTTGGGCACTGACAATTCCGTGGTGTTCTGTGCCTTCTAGTT
GCCAGCCATCTGTTGTTTGCCCCTCCCCCGTGCCTTCCTTGACCCTGGAAGGTGCCACTCCCACTGTC
CTTTCCTAATAAAATGAGGAAATTGCATCGCATTGTCTGAGTAGGTGTCATTCTATTCTGGGGGGTGGG
GTGGGGCAGGACAGCAAGGGGGAGGATTGGGAAGACAATAGCAGGCATGCTGGGGATGCGGTGGGC
TCTATGGGCGGCCGCGTCGGCCGCAGGAACCCCTAGTGATGGAGTTGGCCACTCCCTCTCTGCGCGCT
CGCTCGCTCACTGAGGCCGGGCGACCAAAGGTCGCCCGACGCCCGGGCTTTGCCCGGGCGGCCTCA
GTGAGCGAGCGAGCGCGCAG
bpSV40 NLS amino acid sequence, SEQ ID NO: 58
KRTADGSEFESPKKKRKV
WPRE3, SEQ ID NO: 59
AATCAACCTCTGGATTACAAAATTTGTGAAAGATTGACTGGTATTCTTAACTATGTTGCTCCTTTTACGC
TATGTGGATACGCTGCTTTAATGCCTTTGTATCATGCTATTGCTTCCCGTATGGCTTTCATTTTCTCCTCC
TTGTATAAATCCTGGTTAGTTCTTGCCACGGCGGAACTCATCGCCGCCTGCCTTGCCCGCTGCTGGAC
AGGGGCTCGGCTGTTGGGCACTGACAATTCCGTGGTGTT
MCEP2 mRNA (NM_004992.4) full length, SEQ ID NO: 60
gcgcgcgctccctcctctcggagagagggctgtggtaaaagccgtccggaaaatggccgccgccgccgccgccgcgccgagcggaggaggaggagga
ggcgaggaggagagactgctccataaaaatacagactcaccagttcctgctttgatgtgacatgtgactccccagaatacaccttgcttctg
tagaccagctccaacaggattccatggtagctgggatgttagggctcagggaagaaaagtcagaagaccaggacctccagggcctcaaggacaaac
ccctcaagtttaaaaaggtgaagaaagataagaaagaagagaaagagggcaagcatgagcccgtgcagccatcagcccaccactctgctgag
cccgcagaggcaggcaaagcagagacatcagaagggtcaggctccgccccggctgtgccggaagcttctgcctcccccaaacagcggcgctccat
catccgtgaccggggacccatgtatgatgaccccaccctgcctgaaggctggacacggaagcttaagcaaaggaaatctggccgctctgctgg
gaagtatgatgtgtatttgatcaatccccagggaaaagcctttcgctctaaagtggagttgattgcgtacttcgaaaaggtaggcgacacatc
cctggaccctaatgattttgacttcacggtaactgggagagggagcccctcccggcgagagcagaaaccacctaagaagcccaaatctcccaaag
ctccaggaactggcagaggccggggacgccccaaagggagcggcaccacgagacccaaggcggccacgtcagagggtgtgcaggtgaaaagggtc
ctggagaaaagtcctgggaagctccttgtcaagatgccttttcaaacttcgccagggggcaaggctgaggggggtggggccaccacatccacccaggtc
atggtgatcaaacgccccggcaggaagcgaaaagctgaggccgaccctcaggccattcccaagaaacggggccgaaagccggggagtgtggtggcag
ccgctgccgccgaggccaaaaagaaagccgtgaaggagtcttctatccgatctgtgcaggagaccgtactccccatcaagaagcgcaagacccggg
agacggtcagcatcgaggtcaaggaagtggtgaagcccctgctggtgtccaccctcggtgagaagagcgggaaaggactgaagacctgtaagagc
cctgggcggaaaagcaaggagagcagccccaaggggcgcagcagcagcgcctcctcaccccccaagaaggagcaccaccaccatcaccaccactcag
agtccccaaaggcccccgtgccactgctcccacccctgcccccacctccacctgagcccgagagctccgaggaccccaccagcccccctgagcccc
aggacttgagcagcagcgtctgcaaagaggagaagatgcccagaggaggctcactggagagcgacggctgccccaaggagccagctaagactcagcc
cgcggttgccaccgccgccacggccgcagaaaagtacaaacaccgaggggagggagagcgcaaagacattgtttcatcctccatgccaaggccaaac
agagaggagcctgtggacagccggacgcccgtgaccgagagagttagctgactttacacggagcggattgcaaagcaaaccaacaagaataaaggcag
ctgttgtctcttctccttatgggtagggctctgacaaagcttcccgattaactgaaataaaaaatatttttttttctttcagtaaacttagagtttcgt
ggcttcaggggggagtagttggagcattggggatgtttttcttaccgacaagcacagtcaggttgaagacctaaccagggccagaagtagcttt
gcacttttctaaactaggctccttcaacaaggcttgctgcagatactactgaccagacaagctgttgaccaggcacctcccctcccgcccaaacc
tttcccccatgtggtcgttagagacagagcgacagagcagttgagaggacactcccgttttcggtgccatcagtgccccgtctacagctcccccagctc
cccccacctcccccactcccaaccacgttgggacagggaggtgtgaggcaggagagacagttggattctttagagaagatggatatgaccagtggc
tatggcctgtgcgatcccacccgtggtggctcaagtctggccccacaccagccccaatccaaaactggcaaggacgcttcacaggacaggaaagtg
gcacctgtctgctccagctctggcatggctaggaggggggagtcccttgaactactgggtgtagactggcctgaaccacaggagaggatggcc
cagggtgaggtggcatggtccattctcaagggacgtcctccaacgggtggcgctagaggccatggaggcagtaggacaaggtgcaggcag
gctggcctggggtcaggccgggcagagcacagcggggtgagagggattcctaatcactcagagcagtctgtgacttagtggacaggggagggg
gcaaagggggaggagaagaaaatgttcttccagttactttccaattctcctttagggacagcttagaattatttgcactattgagtcttcatg
ttcccacttcaaaacaaacagatgctctgagagcaaactggcttgaattggtgacatttagtccctcaagccaccagatgtgacagtgttgagaa
ctacctggatttgtatatatacctgcgcttgttttaaagtgggctcagcacatagggttcccacgaagctccgaaactctaagtgtttgctgca
attttataaggacttcctgattggtttctcttctccccttccatttctgccttttgttcatttcatcctttcacttctttcccttcctccatcctcc
tccttcctagttcatcccttctcttccaggcagccgcggtgcccaaccacacttgtcggctccagtccccagaactctgcctgccctttgtcctcc
tgctgccagtaccagccccaccctgttttgagccctgaggaggccttgggctctgctgagtccgacctggcctgtctgtgaagagcaagagagcagc
aaggtcttgctctcctaggtagccccctcttccctggtaagaaaaagcaaaaggcatttcccaccctgaacaacgagccttttcacccttctactcta
gagaagtggactggaggagctgggcccgatttggtagttgaggaaagcacagaggcctcctgtggcctgccagtcatcgagtggcccaacagg
ggctccatgccagccgaccttgacctcactcagaagtccagagtctagcgtagtgcagcagggcagtagcggtaccaatgcagaactcccaagacccga
gctgggaccagtacctgggtccccagcccttcctctgctcccccttttccctcggagttcttcttgaatggcaatgttttgcttttgctcg
atgcagacagggggccagaacaccacacatttcactgtctgtctggtccatagctgtggtgtaggggcttagaggcatgggcttgctgtgggttt
ttaattgatcagttttcatgtgggatcccatctttttaacctctgttcaggaagtccttatctagctgcatatcttcatcatattggtatatcct
tttctgtgtttacagagatgtctcttatatctaaatctgtccaactgagaagtaccttatcaaagtagcaaatgagacagcagtcttatgct
tccagaaacacccacaggcatgtcccatgtgagctgctgccatgaactgtcaagtgtgtgttgtcttgtgtatttcagttattgtccctggct
tccttactatggtgtaatcatgaaggagtgaaacatcatagaaactgtctagcacttccttgccagtctttagtgatcaggaaccatagttgacagt
tccaatcagtagcttaagaaaaaaccgtgtttgtctcttctggaatggttagaagtgagggagtttgccccgttctgtttgtagagtctcatagttg
gactttctagcatatatgtgtccatttccttatgctgtaaaagcaagtcctgcaaccaaactcccatcagcccaatccctgatccctgatcccttccac
ctgctctgctgatgacccccccagcttcacttctgactcttccccaggaagggaaggggggtcagaagagagggtgagtcctccagaactcttcctcc
aaggacagaaggctcctgcccccatagtggcctcgaactcctggcactaccaaaggacacttatccacgagagcgcagcatccgaccaggttgtcactg
agaagatgtttattttggtcagttgggtttttatgtattatacttagtcaaatgtaatgtggcttctggaatcattgtccagagctgcttccccgtcacc
tgggcgtcatctggtcctggtaagaggagtgcgtggcccaccaggcccccctgtcacccatgacagttcattcagggccgatggggcagtcgtggttgg
gaacacagcatttcaagcgtcactttatttcattcgggccccacctgcagctccctcaaagaggcagttgcccagcctctttcccttccagtttattcc
agagctgccagtggggcctgaggctccttagggttttctctctatttccccctttcttcctcattccctcgtctttcccaaaggcatcacgagtcagtc
gcctttcagcaggcagccttggcggtttatcgccctggcaggcaggggccctgcagctctcatgctgcccctgccttggggtcaggttgacaggaggtt
ggagggaaagccttaagctgcaggattctcaccagctgtgtccggcccagttttggggtgtgacctcaatttcaattttgtctgtacttgaacattatg
aagatgggggcctctttcagtgaatttgtgaacagcagaattgaccgacagctttccagtacccatggggctaggtcattaaggccacatccacag
tctcccccacccttgttccagttgttagttactacctcctctcctgacaatactgtatgtcgtcgagctccccccaggtctacccctcccggccctgcct
gctggtgggcttgtcatagccagtgggattgccggtcttgacagctcagtgagctggagatacttggtcacagccaggcgctagcacagctcc
cttctgttgatgctgtattcccatatcaaaagacacaggggacacccagaaacgccacatcccccaatccatcagtgccaaactagccaacggcccc
agcttctcagctcgctggatggcggaagctgctactcgtgagcgccagtgcgggtgcagacaatcttctgttgggtggcatcattccaggcccgaag
catgaacagtgcacctgggacagggagcagccccaaattgtcacctgcttctctgcccagcttttcattgctgtgacagtgatggcgaaagagggtaa
taaccagacacaaactgccaagttgggtggagaaaggagtttctttagctgacagaatctctgaattttaaatcacttagtaagcggctcaagccc
aggagggagcagagggatacgagcggagtcccctgcgcgggaccatctggaattggtttagcccaagtggagcctgacagccagaactctgtgtccccc
gtctaaccacagctccttttccagagcattccagtcaggctctctgggctgactgggccaggggaggttacaggtaccagttctttaagaagatc
tttgggcatatacatttttagcctgtgtcattgccccaaatggattcctgtttcaagttcacacctgcagattctaggacctgtgtcctagacttcag
ggagtcagctgtttctagagttcctaccatggagtgggtctggaggacctgcccggtgggggggcagagccctgctccctccgggtcttcctactctt
ctctctgctctgacgggatttgttgattctctccattttggtgtctttctcttttagatattgtatcaatctttagaaaaggcatagtctactt
gttataaatcgttaggatactgcctcccccagggtctaaaattacatattagaggggaaaagctgaacactgaagtcagttctcaacaatt
tagaaggaaaacctagaaaacatttggcagaaaattacatttcgatgtttttgaatgaatacgagcaagcttttacaacagtgctgatctaaaaata
cttagcacttggcctgagatgcctggtgagcattacaggcaaggggaatctggaggtagccgacctgaggacatggcttctgaacctgtctt
ttgggagtggtatggaaggtggagcgttcaccagtgacctggaaggcccagcaccaccctccttcccactcttctcatcttgacagagcctgcc
ccagcgctgacgtgtcaggaaaacacccagggaactaggaaggcacttctgcctgaggggcagcctgccttgcccactcctgctctgctcgcctcgga
tcagctgagccttctgagctggcctctcactgcctccccaaggccccctgcctgccctgtcaggaggcagaaggaagcaggtgtgagggcagtgca
aggagggagcacaacccccagctcccgctccgggctccgacttgtgcacaggcagagcccagaccctggaggaaatcctacctttgaattcaa
gaacatttggggaatttggaaatctctttgcccccaaacccccattctgtcctacctttaatcaggtcctgctcagcagtgagagcagatgag
gtgaaaaggccaagaggtttggctcctgcccactgatagcccctctccccgcagtgtttgtgtgtcaagtggcaaagctgttcttcctggtgaccct
gattatatccagtaacacatagactgtgcgcataggcctgctttgtctcctctatcctgggcttttttttgctttttagttttgctttta
gtttttctgtcccttttatttaacgcaccgactagacacacaaagcagttgaatttttatatatatatctgtatattgcacaattataaactcatttt
gcttgtggctccacacacacaaaaaaagacctgttaaaattatacctgttgcttaattacaatatttctgataaccatagcataggacaaggg
aaaataaaaaaagaaaaaaaagaaaaaaaaacgacaaatctgtctgctggtcacttcttctgtccaagcagattcgtggtcttttcctcgcttct
ttcaagggctttcctgtgccaggtgaaggaggctccaggcagcacccaggttttgcactcttgtttctcccgtgcttgtgaaagaggtcccaagg
ttctgggtgcaggagcgctcccttgacctgctgaagtccggaacgtagtcggcacagcctggtcgccttccacctctgggagctggagtccactgggg
tggcctgactcccccagtccccttcccgtgacctggtcagggtgagcccatgtggagtcagcctcgcaggcctccctgccagtagggtccgagtgtg
tttcatccttcccactctgtcgagcctgggggctggagcggagacgggaggcctggcctgtctcggaacctgtgagctgcaccaggtagaacgc
cagggaccccagaatcatgtgcgtcagtccaaggggtcccctccaggagtagtgaagactccagaaatgtccctttcttctcccccatcctacgagtaa
ttgcatttgcttttgtaattcttaatgagcaatatctgctagagagtttagctgtaacagttctttttgatcatctttttttaataattagaaac
accaaaaaaatccagaaacttgttcttccaaagcagagagcattataatcaccagggccaaaagcttccctccctgctgtcattgcttcttc
tgaggcctgaatccaaaagaaaaacagccataggccctttcagtggccgggctacccgtgagcccttcggaggaccagggctggggcagcctc
tgggcccacatccggggccagctccggcgtgtgttcagtgttagcagtgggtcatgatgctctttcccacccagcctgggataggggcagagga
ggcgaggaggccgttgccgctgatgtttggccgtgaacaggtgggtgtctgcgtgcgtccacgtgcgtgttttctgactgacatgaaatcgacgc
ccgagttagcctcacccggtgacctctagccctgcccggatggagcggggcccacccggttcagtgtttctggggagctggacagtggagtgcaaaag
gcttgcagaacttgaagcctgctccttcccttgctaccacggcctcctttccgtttgatttgtcactgcttcaatcaataacagccgctccagag
tcagtagtcaatgaatatatgaccaaatatcaccaggactgttactcaatgtgtgccgagcccttgcccatgctgggctcccgtgtatctg
gacactgtaacgtgtgctgtgtttgctccccttccccttccttctttgccctttacttgtctttctggggtttttctgtttgggtttggtttggtt
tttatttctccttttgtgttccaaacatgaggttctctctactggtcctcttaactgtggtgttgaggcttatatttgtgtaatttttggtggg
tgaaaggaattttgctaagtaaatctcttctgtgtttgaactgaagtctgtattgtaactatgtttaaagtaattgttccagagacaaatattt
ctagacactttttctttacaaacaaaagcattcggagggagggggatggtgactgagatgagaggggagagctgaacagatgacccctgcccagatca
gccagaagccacccaaagcagtggagcccaggagtcccactccaagccagcaagccgaatagctgatgtgttgccactttccaagtcactgcaaaacca
ggttttgttccgcccagtggattcttgttttgcttcccctccccccgagattattaccaccatcccgtgcttttaaggaaaggcaagattgatgtt
tccttgaggggagccaggaggggatgtgtgtgtgcagagctgaagagctggggagaatggggctgggcccacccaagcaggaggctgggacgctctg
ctgtgggcacaggtcaggctaatgttggcagatgcagctcttcctggacaggccaggtggtgggcattctctctccaaggtgtgccccgtgggcat
tactgtttaagacacttccgtcacatcccaccccatcctccagggctcaacactgtgacatctctattccccaccctccccttcccagggcaataaaat
gaccatggagggggcttgcactctcttggctgtcacccgatcgccagcaaaacttagatgtgagaaaaccccttcccattccatggcgaaa
acatctccttagaaaagccattaccctcattaggcatggttttgggctcccaaaacacctgacagcccctccctcctctgagaggcggagagt
gctgactgtagtgaccattgcatgccgggtgcagcatctggaagagctaggcagggtgtctgccccctcctgagttgaagtcatgctccc
ctgtgccagcccagaggccgagagctatggacagcattgccagtaacacaggccaccctgtgcagaagggagctggctccagcctggaaacctgtc
tgaggttgggagaggtgcacttggggcacagggagaggccgggacacacttagctggagatgtctctaaaagccctgtatcgtattcaccttca
gtttttgtgttttgggacaattactttagaaaataagtaggtcgttttaaaaacaaaaattattgattgcttttttgtagtgttcagaaaaaag
gttctttgtgtatagccaaatgactgaaagcactgatatatttaaaaacaaaaggcaatttattaaggaaatttgtaccatttcagtaaacctgtctg
aatgtacctgtatacgtttcaaaaacaccccccccccactgaatccctgtaacctatttattatataaagagtttgccttataaatttacataaaaatg
tccgtttgtgtcttttgttgtaaaaatcaagtgattttttcataaggttcttttactattggaaaagatgggcagcacgcagttttatttta
tttttgtaagttttttaatacatgtgaaagcaaagaatactcagcatgcctttctaagtgacgcgtttgcaccttttgttgggaagtactgtatcctgt
gctgttagcattctcgataaatctctctgtgaaagtga
hfCas13f.v3 (Cas13f.1-D160A + D642A + Y666A + Y677A) (hfCas13f) amino acid sequence, SEQ ID NO: 61
MNGIELKKEEAAFYENQAELNLKAIEDNIFDKERRKTLLNNPQILAKMENFIFNFRDVTKNAKGEIDCLLL
KLRELRNFYSHYVHKRDVRELSKGEKPILEKYYQFAIESTGSENVKLEIIENDAWLADAGVLFFLCIFLKK
SQANKLISGISGFKRNDATGQPRRNLFTYFSIREGYKVVPEMQKHFLLFSLVNHLSNQDDYIEKAHQPYDI
GEGLFFHRIASTFLNISGILRNMKFYTYQSKRIVEQRGELKREKDIFAWEEPFQGNSYFEINGHKGVIGEDE
LKELCYAFLIGNQDANKVEGRITQFLEKFRNANSVQQVKDDEMLKPEYFPANYFAESGVGRIKDRVLNRL
NKAIKSNKAKKGEIIAYDKMREVMAFINNSLPVDEKLKPKDYKRYLGMVRFWDREKDNIKREFETKEWS
KYLPSNFWTAKNLERVYGLAREKNAELFNKLKADVEKMDERELEKYQKINDAKDLANLRRLASDFGVK
WEEKDWDEYSGQIKKQITDSQKLTIMKQRITAGLKKKHGIENLNLRITIDINKSRKAVLNRIAIPRGFVKRH
ILGWQESEKVSKKIREAECEILLSKEYEELSKQFFQSKDYDKMTRINGLYEKNKLIALMAVYLMGQLRILF
KEHTKLADITKTTVDFKISDKVTVKIPFSNAPSLVYTMSSKAVDNIGNYGFSNKDKDKPILGKIDVIEKQR
MEFIKEVLGFEKYLFDDKIIDKSKFADTATHISFAEIVEELVEKGWDKDRLTKLKDARNKALHGEILTGTSF
DETKSLINELKK
hfCas13f.v3 (Cas13f.1-D160A + D642A + Y666A + Y677A) (hfCas13f) coding sequence, SEQ ID NO: 62
ATGAATGGCATCGAGCTGAAGAAGGAAGAAGCCGCCTTCTACTTCAATCAGGCCGAGCTGAACCTGA
AGGCCATTGAGGACAACATCTTCGACAAGGAGAGAAGAAAGACACTGCTGAACAACCCCCAGATCC
TGGCCAAGATGGAGAACTTTATCTTCAATTTCCGGGACGTGACCAAGAACGCCAAGGGCGAAATCGA
CTGCCTGCTGCTGAAGCTGAGAGAGCTGCGGAACTTTTACAGCCACTACGTGCACAAGCGGGACGTC
AGAGAACTGAGCAAGGGCGAGAAGCCGATCCTGGAGAAGTACTACCAGTTCGCCATCGAATCCACC
GGCTCTGAGAACGTGAAGCTCGAAATCATCGAAAACGACGCCTGGCTGGCCGACGCCGGCGTGCTG
TTCTTCCTGTGCATCTTCCTGAAGAAGAGCCAGGCAAACAAGCTGATCAGCGGCATCAGCGGCTTCA
AGAGAAACGACGCCACCGGCCAGCCTCGGAGAAACCTGTTCACCTACTTCTCCATCCGGGAGGGCTA
CAAGGTGGTGCCCGAAATGCAGAAGCACTTCCTGCTGTTCTCCCTGGTGAACCACCTGAGCAACCAG
GACGATTATATCGAAAAGGCCCACCAGCCCTACGACATCGGCGAGGGCCTCTTCTTCCACCGGATTGC
CAGCACCTTCCTGAACATCTCCGGAATCCTGAGAAACATGAAGTTCTACACCTATCAGAGCAAGAGA
CTGGTGGAGCAGAGAGGCGAGCTGAAGCGGGAAAAGGACATCTTCGCCTGGGAAGAACCGTTTCAG
GGCAATTCCTACTTTGAGATCAACGGCCACAAGGGCGTGATTGGCGAGGACGAGCTGAAGGAGCTGT
GCTACGCCTTCCTGATCGGCAACCAGGACGCCAACAAGGTGGAGGGCCGGATCACCCAGTTCCTGGA
GAAGTTCAGAAACGCCAACAGCGTGCAGCAGGTGAAGGACGACGAGATGCTGAAGCCTGAATATTT
CCCCGCCAACTACTTTGCCGAGAGCGGCGTGGGCCGGATCAAGGACCGGGTGCTGAACAGACTGAA
CAAGGCCATCAAGAGCAACAAGGCCAAGAAGGGCGAGATCATCGCCTATGACAAGATGAGAGAAGT
GATGGCTTTCATCAATAACTCTCTGCCCGTGGACGAGAAGCTGAAGCCCAAGGATTACAAGAGATACC
TGGGCATGGTGAGATTCTGGGATAGAGAAAAGGACAATATCAAGCGCGAGTTCGAAACGAAGGAGT
GGAGCAAGTATCTGCCCTCCAACTTCTGGACCGCCAAGAACCTGGAGAGAGTGTACGGACTGGCCCG
GGAAAAGAACGCAGAGCTGTTTAACAAGCTGAAGGCCGACGTGGAGAAGATGGACGAAAGAGAGC
TGGAAAAGTATCAGAAGATCAACGACGCCAAGGATCTGGCCAACCTGCGGCGGCTGGCCAGCGACTT
CGGAGTGAAGTGGGAGGAGAAGGATTGGGACGAGTACTCCGGCCAGATCAAGAAGCAGATCACAGA
TTCCCAGAAGCTGACCATCATGAAGCAGAGAATCACAGCCGGCCTGAAGAAGAAGCACGGCATCGA
AAACCTGAACCTGAGGATCACCATCGACATCAACAAGTCCAGAAAGGCCGTGCTGAATCGGATCGCC
ATCCCCAGAGGATTTGTGAAGCGGCACATCCTGGGCTGGCAGGAATCCGAGAAGGTGAGCAAGAAG
ATCAGAGAAGCCGAATGCGAGATTCTGCTGAGCAAGGAGTACGAGGAGCTGAGCAAGCAGTTCTTTC
AGAGCAAGGACTACGACAAGATGACCCGCATCAACGGCCTGTACGAGAAGAATAAGCTGATCGCCCT
GATGGCCGTGTATCTGATGGGGCAGCTGAGAATCCTGTTCAAGGAGCACACCAAGCTGGCCGACATC
ACCAAGACCACCGTGGATTTCAAGATCAGCGACAAGGTGACCGTGAAGATCCCCTTCTCCAACGCTC
CCTCCCTGGTGTACACCATGAGCAGCAAGGCCGTGGACAATATCGGCAACTACGGCTTCAGCAACAA
GGACAAGGATAAGCCCATTCTGGGCAAGATCGACGTGATCGAGAAGCAGCGGATGGAGTTTATCAAG
GAGGTGCTGGGATTCGAGAAGTACCTGTTTGACGATAAGATCATCGACAAGAGCAAGTTCGCCGACA
CCGCCACCCACATCAGCTTTGCCGAAATCGTGGAAGAACTGGTGGAGAAGGGCTGGGACAAGGACC
GGCTGACGAAGCTGAAGGATGCCCGGAACAAGGCCCTGCACGGCGAGATCCTGACCGGCACCAGCT
TCGACGAGACAAAGTCCCTGATCAACGAGCTGAAGAAG
mMECP2 promoter, SEQ ID NO: 63
AGCTGAATGGGGTCCGCCTCTTTTCCCTGCCTAAACAGACAGGAACTCCTGCCAATTGAGGGC
GTCACCGCTAAGGCTCCGCCCCAGCCTGGGCTCCACAACCAATGAAGGGTAATCTCGACAAAG
AGCAAGGGGTGGGGCGCGGGCGCGCAGGTGCAGCAGCACACAGGCTGGTCGGGAGGGCGGG
GCGCGACGTCTGCCGTGCGGGGTCCCGGCATCGGTTGCGCGC
bpSV40 NLS coding sequence 1, SEQ ID NO: 64
AAACGGACAGCCGACGGAAGCGAGTTCGAGTCACCCAAGAAGAAGCGGAAGGTG
bpSV40 NLS coding sequence 2, SEQ ID NO: 65
AAAAGAACCGCCGACGGCAGCGAATTCGAGTCACCCAAGAAGAAGCGGAAGGTG
AAV9 transgene plasmid- 8 hfCas13f.v2.5 9 -gRNA 9&3 ITR-to-ITR, SEQ ID NO: 66
CTGCGCGCTCGCTCGCTCACTGAGGCCGCCCGGGCAAAGCCCGGGCGTCGGGCGACCTTTGGTCGCC
CGGCCTCAGTGAGCGAGCGAGCGCGCAGAGAGGGAGTGGCCAACTCCATCACTAGGGGTTCCTGCG
GCCATCGATACTAGTGAGGGCCTATTTCCCATGATTCCTTCATATTTGCATATACGATACAAGGCTGTTA
GAGAGATAATTGGAATTAATTTGACTGTAAACACAAAGATATTAGTACAAAATACGTGACGTAGAAAG
TAATAATTTCTTGGGTAGTTTGCAGTTTTAAAATTATGTTTTAAAATGGACTATCATATGCTTACCGTAAC
TTGAAAGTATTTCGATTTCTTGGCTTTATATATCTTGTGGAAAGGACGAAACACCGGCTGTGATAGAC
CTCGATTTGTGGGGTAGTAACAGCGTTACCGTGAAGTCAAAATCATTAGGGTCCGCTGTGATAGAC
CTCGATTTGTGGGGTAGTAACAGCGGGCTTCACCACTTCCTTGACCTCGATGCTGCTGTGATAGAC
CTCGATTTGTGGGGTAGTAACAGCTTTTTTTCCAGCTGAATGGGGTCCGCCTCTTTTCCCTGCCTAA
ACAGACAGGAACTCCTGCCAATTGAGGGCGTCACCGCTAAGGCTCCGCCCCAGCCTGGGCTCCACAA
CCAATGAAGGGTAATCTCGACAAAGAGCAAGGGGTGGGGCGCGGGCGCGCAGGTGCAGCAGCACAC
AGGCTGGTCGGGAGGGCGGGGCGCGACGTCTGCCGTGCGGGGTCCCGGCATCGGTTGCGCGCACCG
GTGCCACCATGAAACGGACAGCCGACGGAAGCGAGTTCGAGTCACCCAAGAAGAAGCGGAAGGTG
AATGGCATCGAGCTGAAGAAGGAAGAAGCCGCCTTCTACTTCAATCAGGCCGAGCTGAACCTGAAGG
CCATTGAGGACAACATCTTCGACAAGGAGAGAAGAAAGACACTGCTGAACAACCCCCAGATCCTGG
CCAAGATGGAGAACTTTATCTTCAATTTCCGGGACGTGACCAAGAACGCCAAGGGCGAAATCGACTG
CCTGCTGCTGAAGCTGAGAGAGCTGCGGAACTTTTACAGCCACTACGTGCACAAGCGGGACGTCAG
AGAACTGAGCAAGGGCGAGAAGCCGATCCTGGAGAAGTACTACCAGTTCGCCATCGAATCCACCGGC
TCTGAGAACGTGAAGCTCGAAATCATCGAAAACGACGCCTGGCTGGCCGACGCCGGCGTGCTGTTCT
TCCTGTGCATCTTCCTGAAGAAGAGCCAGGCAAACAAGCTGATCAGCGGCATCAGCGGCTTCAAGAG
AAACGACGCCACCGGCCAGCCTCGGAGAAACCTGTTCACCTACTTCTCCATCCGGGAGGGCTACAAG
GTGGTGCCCGAAATGCAGAAGCACTTCCTGCTGTTCTCCCTGGTGAACCACCTGAGCAACCAGGACG
ATTATATCGAAAAGGCCCACCAGCCCTACGACATCGGCGAGGGCCTCTTCTTCCACCGGATTGCCAGC
ACCTTCCTGAACATCTCCGGAATCCTGAGAAACATGAAGTTCTACACCTATCAGAGCAAGAGACTGGT
GGAGCAGAGAGGCGAGCTGAAGCGGGAAAAGGACATCTTCGCCTGGGAAGAACCGTTTCAGGGCAA
TTCCTACTTTGAGATCAACGGCCACAAGGGCGTGATTGGCGAGGACGAGCTGAAGGAGCTGTGCTAC
GCCTTCCTGATCGGCAACCAGGACGCCAACAAGGTGGAGGGCCGGATCACCCAGTTCCTGGAGAAG
TTCAGAAACGCCAACAGCGTGCAGCAGGTGAAGGACGACGAGATGCTGAAGCCTGAATATTTCCCCG
CCAACTACTTTGCCGAGAGCGGCGTGGGCCGGATCAAGGACCGGGTGCTGAACAGACTGAACAAGG
CCATCAAGAGCAACAAGGCCAAGAAGGGCGAGATCATCGCCTATGACAAGATGAGAGAAGTGATGG
CTTTCATCAATAACTCTCTGCCCGTGGACGAGAAGCTGAAGCCCAAGGATTACAAGAGATACCTGGGC
ATGGTGAGATTCTGGGATAGAGAAAAGGACAATATCAAGCGCGAGTTCGAAACGAAGGAGTGGAGC
AAGTATCTGCCCTCCAACTTCTGGACCGCCAAGAACCTGGAGAGAGTGTACGGACTGGCCCGGGAAA
AGAACGCAGAGCTGTTTAACAAGCTGAAGGCCGACGTGGAGAAGATGGACGAAAGAGAGCTGGAA
AAGTATCAGAAGATCAACGACGCCAAGGATCTGGCCAACCTGCGGCGGCTGGCCAGCGACTTCGGA
GTGAAGTGGGAGGAGAAGGATTGGGACGAGTACTCCGGCCAGATCAAGAAGCAGATCACAGATTCC
CAGAAGCTGACCATCATGAAGCAGAGAATCACAGCCGGCCTGAAGAAGAAGCACGGCATCGAAAAC
CTGAACCTGAGGATCACCATCGACATCAACAAGTCCAGAAAGGCCGTGCTGAATCGGATCGCCATCC
CCAGAGGATTTGTGAAGCGGCACATCCTGGGCTGGCAGGAATCCGAGAAGGTGAGCAAGAAGATCA
GAGAAGCCGAATGCGAGATTCTGCTGAGCAAGGAGTACGAGGAGCTGAGCAAGCAGTTCTTTCAGA
GCAAGGACTACGACAAGATGACCCGCATCAACGGCCTGTACGAGAAGAATAAGCTGATCGCCCTGAT
GGCCGTGTATCTGATGGGGCAGCTGAGAATCCTGTTCAAGGAGCACACCAAGCTGGCCGACATCACC
AAGACCACCGTGGATTTCAAGATCAGCGACAAGGTGACCGTGAAGATCCCCTTCTCCAACGCTCCCT
CCCTGGTGTACACCATGAGCAGCAAGGCCGTGGACAATATCGGCAACTACGGCTTCAGCAACAAGGA
CAAGGATAAGCCCATTCTGGGCAAGATCGACGTGATCGAGAAGCAGCGGATGGAGTTTATCAAGGAG
GTGCTGGGATTCGAGAAGTACCTGTTTGACGATAAGATCATCGACAAGAGCAAGTTCGCCGACACCG
CCACCCACATCAGCTTTGCCGAAATCGTGGAAGAACTGGTGGAGAAGGGCTGGGACAAGGACCGGC
TGACGAAGCTGAAGGATGCCCGGAACAAGGCCCTGCACGGCGAGATCCTGACCGGCACCAGCTTCG
ACGAGACAAAGTCCCTGATCAACGAGCTGAAGAAGCCCAAGAAGAAGCGGAAGGTGTTCTGGCGGC
TCAAAAAGAACCGCCGACGGCAGCGAATTCGAGTCACCCAAGAAGAAGCGGAAGGTGGTTAGAAGC
TTAATCAACCTCTGGATTACAAAATTTGTGAAAGATTGACTGGTATTCTTAACTATGTTGCTCCTTTTAC
GCTATGTGGATACGCTGCTTTAATGCCTTTGTATCATGCTATTGCTTCCCGTATGGCTTTCATTTTCTCCT
CCTTGTATAAATCCTGGTTAGTTCTTGCCACGGCGGAACTCATCGCCGCCTGCCTTGCCCGCTGCTGGA
CAGGGGCTCGGCTGTTGGGCACTGACAATTCCGTGGTGTTCTGTGCCTTCTAGTTGCCAGCCATCTGT
TGTTTGCCCCTCCCCCGTGCCTTCCTTGACCCTGGAAGGTGCCACTCCCACTGTCCTTTCCTAATAAAA
TGAGGAAATTGCATCGCATTGTCTGAGTAGGTGTCATTCTATTCTGGGGGGTGGGGTGGGGCAGGACA
GCAAGGGGGAGGATTGGGAAGACAATAGCAGGCATGCTGGGGATGCGGTGGGCTCTATGGGCGGCC
GCAGGAACCCCTAGTGATGGAGTTGGCCACTCCCTCTCTGCGCGCTCGCTCGCTCACTGAGGCCGGG
CGACCAAAGGTCGCCCGACGCCCGGGCTTTGCCCGGGCGGCCTCAGTGAGCGAGCGAGCGCGCAG

1. A guide nucleic acid comprising:
(1) a scaffold sequence capable of forming a complex with a nucleic acid programmable RNA nuclease (napRNAn), and
(2) a guide sequence capable of hybridizing to a target sequence of human MECP2 mRNA, thereby guiding the complex to the human MECP2 mRNA.

2. A system or composition comprising:
(1) a guide nucleic acid, or a polynucleotide encoding the guide nucleic acid, comprising:
(a) a scaffold sequence capable of forming a complex with a nucleic acid programmable RNA nuclease (napRNAn), and
(b) a guide sequence capable of hybridizing to a target sequence of human MECP2 mRNA, thereby guiding the complex to the human MECP2 mRNA; and
(2) the napRNAn or a polynucleotide encoding the napRNAn.

3. A recombinant adeno-associated virus (rAAV) vector genome comprising:
(a) a first polynucleotide sequence comprising a first sequence encoding a first guide nucleic acid comprising:
(1) a scaffold sequence capable of forming a complex with a nucleic acid programmable RNA nuclease (napRNAn), and
(2) a first guide sequence capable of hybridizing to a first target sequence of human MECP2 mRNA, thereby guiding the complex to the human MECP2 mRNA; and
(b) a second polynucleotide sequence comprising a sequence encoding the napRNAn,
wherein the rAAV vector genome is adapted to be encapsulated into a recombinant AAV particle.

4. The rAAV vector genome of claim 3, wherein the first polynucleotide sequence further comprises a second sequence encoding a second guide nucleic acid comprising:
(1) a or the scaffold sequence capable of forming a complex with a napRNAn, and
(2) a second guide sequence capable of hybridizing to a second target sequence of human MECP2 mRNA, thereby guiding the complex to the human MECP2 mRNA.

5. The rAAV vector genome of claim 4, wherein the first polynucleotide sequence further comprises a third sequence encoding a third guide nucleic acid comprising:
(1) a or the scaffold sequence capable of forming a complex with a napRNAn, and
(2) a third guide sequence capable of hybridizing to a third target sequence of human MECP2 mRNA, thereby guiding the complex to the human MECP2 mRNA.

6. The rAAV vector genome of claim 5, wherein the first polynucleotide sequence further comprises a fourth sequence encoding a fourth guide nucleic acid comprising:
(1) a or the scaffold sequence capable of forming a complex with a napRNAn, and
(2) a fourth guide sequence capable of hybridizing to a fourth target sequence of human MECP2 mRNA, thereby guiding the complex to the human MECP2 mRNA.

7. The guide nucleic acid, the system or composition, or the rAAV vector genome of any of the preceding claims, wherein the guide nucleic acid (e.g., the guide nucleic acid, the first guide nucleic acid, the second guide nucleic acid, the third guide nucleic acid, the fourth guide nucleic acid, and so on) is an RNA.

8. The guide nucleic acid, the system or composition, or the rAAV vector genome of any of the preceding claims, wherein said guiding the complex to the human MECP2 mRNA enables the napRNAn to specifically cleave the human MECP2 mRNA.

9. The guide nucleic acid, the system or composition, or the rAAV vector genome of any of the preceding claims, wherein the specific cleavage of the human MECP2 mRNA leads to degradation of the human MECP2 mRNA.

10. The guide nucleic acid, the system or composition, or the rAAV vector genome of any of the preceding claims, wherein the target sequence (e.g., the target sequence, the first target sequence, the second target sequence, the third target sequence, the fourth target sequence, and so on) is located such that the human MECP2 mRNA is specifically cleaved by the napRNAn.

11. The guide nucleic acid, the system or composition, or the rAAV vector genome of claim 17, wherein the human MECP2 mRNA is set in forth in SEQ ID NO: 60.

12. The guide nucleic acid, the system or composition, or the rAAV vector genome of any of the preceding claims, wherein the guide nucleic acid comprises the scaffold sequence 5′ or 3′, and optionally 3′, to the guide sequence (e.g., the guide sequence, the first guide sequence, the second guide sequence, the third guide sequence, the fourth guide sequence, and so on).

13. The guide nucleic acid, the system or composition, or the rAAV vector genome of any of the preceding claims, wherein the guide nucleic acid comprises, from 5′ to 3′, one guide sequence and one scaffold sequence.

14. The guide nucleic acid, the system or composition, or the rAAV vector genome of any of the preceding claims, wherein the guide nucleic acid comprises, from 5′ to 3′, one scaffold sequence, one guide sequence, and one scaffold sequence, wherein the scaffold sequences are the same or different.

15. The guide nucleic acid, the system or composition, or the rAAV vector genome of any of the preceding claims, wherein the guide nucleic acid comprises, from 5′ to 3′, one scaffold sequence, one guide sequence, one scaffold sequence, and one guide sequence, wherein the scaffold sequences are the same or different, and wherein the guide sequences are the same or different.

16. The guide nucleic acid, the system or composition, or the rAAV vector genome of any of the preceding claims, wherein the guide nucleic acid comprises, from 5′ to 3′, one scaffold sequence, one guide sequence, one scaffold sequence, one guide sequence, and one scaffold sequence, wherein the scaffold sequences are the same or different, and wherein the guide sequences are the same or different.

17. The guide nucleic acid, the system or composition, or the rAAV vector genome of any of the preceding claims, wherein the guide nucleic acid comprises, from 5′ to 3′, one scaffold sequence, one guide sequence, one scaffold sequence, one guide sequence, one scaffold sequence, and one guide sequence, wherein the scaffold sequences are the same or different, and wherein the guide sequences are the same or different.

18. The guide nucleic acid, the system or composition, or the rAAV vector genome of any of the preceding claims, wherein the target sequence is at least about 14 nucleotides in length, e.g., about 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more nucleotides in length, or in a length of a numerical range between any of two preceding values, e.g., in a length of from about 16 to about 50 nucleotides; optionally wherein the target sequence is about 30 nucleotides in length.

19. The guide nucleic acid, the system or composition, or the rAAV vector genome of any of the preceding claims, wherein the target sequence comprises, consists essentially of, or consists of at least about 14 contiguous nucleotides of the human MECP2 mRNA (e.g., about 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides of the human MECP2 mRNA, or in a numerical range between any of two preceding values, e.g., from about 14 to about 50 contiguous nucleotides of the human MECP2 mRNA); optionally wherein the target sequence comprises, consists essentially of, or consists of about 30 contiguous nucleotides of the human MECP2 mRNA.

20. The guide nucleic acid, the system or composition, or the rAAV vector genome of any of the preceding claims, wherein the guide sequence is at least about 14 nucleotides in length, e.g., about 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more nucleotides in length, or in a length of a numerical range between any of two preceding values, e.g., in a length of from about 16 to about 50 nucleotides; optionally wherein the guide sequence is about 30 nucleotides in length.

21. The guide nucleic acid, the system or composition, or the rAAV vector genome of any of the preceding claims, wherein the guide sequence is at least about 50%, 55%, 60%, 65%, 70%, 75%, 80%, 85%, 90%, 95%, or 100% (fully), optionally about 100% (fully), complementary to the target sequence; or wherein the guide sequence comprises no mismatch with the target sequence in the first 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides at the 5′ end of the guide sequence.

22. The guide nucleic acid, the system or composition, or the rAAV vector genome of any of the preceding claims, wherein the target sequence comprises, consists essentially of, or consists of (1) a sequence of any one of SEQ ID NOs: 1-10 or a 5′ or 3′ end truncation thereof with 1, 2, 3, 4, 5, or 6, nucleotides truncated at the 5′ or 3′ end; or (2) a sequence having a sequence identity of at least about 70%, 75%, 80%, 85%, 90%, 95%, or 100% to any one of SEQ ID NOs: 1-10 or a 5′ or 3′ end truncation thereof with 1, 2, 3, 4, 5, or 6 nucleotides truncated at the 5′ or 3′ end; or (3) a sequence having at most 1, 2, 3, 4, 5, 6, 7, 8, 9, or 10 nucleotide differences, whether consecutive or not, compared to any one of SEQ ID NOs: 1-10.

23. The guide nucleic acid, the system or composition, or the rAAV vector genome of any of the preceding claims, wherein the guide sequence comprises, consists essentially of, or consists of (1) a sequence of any one of SEQ ID NOs: 17-26 or a 5′ or 3′ end truncation thereof with 1, 2, 3, 4, 5, or 6, nucleotides truncated at the 5′ or 3′ end; or (2) a sequence having a sequence identity of at least about 70%, 75%, 80%, 85%, 90%, 95%, or 100% to any one of SEQ ID NOs: 17-26 or a 5′ or 3′ end truncation thereof with 1, 2, 3, 4, 5, or 6 nucleotides truncated at the 5′ or 3′ end; or (3) a sequence having at most 1, 2, 3, 4, 5, 6, 7, 8, 9, or 10 nucleotide differences, whether consecutive or not, compared to any one of SEQ ID NOs: 17-26.

24. The guide nucleic acid, the system or composition, or the rAAV vector genome of any of the preceding claims, wherein the guide sequence comprises the sequence of or is set forth in SEQ ID NO: 19 or 25.

25. The guide nucleic acid, the system or composition, or the rAAV vector genome of any of the preceding claims, wherein the scaffold sequence has substantially the same secondary structure as the secondary structure of the sequence of SEQ ID NO: 6.

26. The guide nucleic acid, the system or composition, or the rAAV vector genome of any of the preceding claims, wherein the scaffold sequence comprises, consists essentially of, or consists of a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 6; or a sequence having at most 1, 2, 3, 4, 5, 6, 7, 8, 9, or 10 nucleotide differences, whether consecutive or not, compared to the sequence of SEQ ID NO: 6.

27. The guide nucleic acid, the system or composition, or the rAAV vector genome of any of the preceding claims, wherein the guide nucleic acid comprises, consists essentially of, or consists of a sequence having a sequence identity of at least about 80% (e.g., at least about 80%, 85%, 90%, 91%, 92%, 93%, 94%, 95%, 96%, 97%, 98%, 99%, or 100%) to the sequence of any of SEQ ID NOs: 7-16 and 37-40; or a sequence having at most 1, 2, 3, 4, 5, 6, 7, 8, 9, or 10 nucleotide differences, whether consecutive or not, compared to the sequence of any of SEQ ID NOs: 7-16 and 37-40.

28. The guide nucleic acid, the system or composition, or the rAAV vector genome of any of the preceding claims, wherein the guide nucleic acid comprises SEQ ID NO: 37.

29. The rAAV vector genome of any of the preceding claims, wherein the first polynucleotide sequence comprises a promoter operably linked to the sequence encoding the guide nucleic acid.

30. The rAAV vector genome of any of the preceding claims, wherein the promoter is a ubiquitous, tissue-specific, cell-type specific, constitutive, or inducible promoter; optionally selected from a group consisting of a Cbh promoter, a Cba promoter, a pol I promoter, a pol II promoter, a pol III promoter, a T7 promoter, a U6 promoter, a H1 promoter, a retroviral Rous sarcoma virus LTR promoter, a cytomegalovirus (CMV) promoter, a SV40 promoter, a dihydrofolate reductase promoter, a β-actin promoter, an elongation factor 1a short (EFS) promoter, a β glucuronidase (GUSB) promoter, a cytomegalovirus (CMV) immediate-early (Ie) enhancer and/or promoter, a chicken f-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, and a myelin basic protein (MBP) promoter.

31. The rAAV vector genome of any of the preceding claims, wherein the promoter is a U6 promoter.

32. The rAAV vector genome of any of the preceding claims, wherein the promoter comprises, consists essentially of, or consists of a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 49; or a sequence having at most 1, 2, 3, 4, 5, 6, 7, 8, 9, or 10 nucleotide differences, whether consecutive or not, compared to the sequence of SEQ ID NO: 49.

33. The rAAV vector genome of any of the preceding claims, wherein the first polynucleotide sequence comprises, from 5′ to 3′, a promoter of SEQ ID NO: 49, and a sequence encoding the guide nucleic acid of any one of SEQ ID NOs: 7-16 and 37-40.

34. The guide nucleic acid, the system or composition, or the rAAV vector genome of any of the preceding claims, wherein the napRNAn is a Class 2, Type VI CRISPR-associated protein (Cas13).

35. The guide nucleic acid, the system or composition, or the rAAV vector genome of any of the preceding claims, wherein the Cas13 is a Cas13a (C2c2), Cas13b (such as, Cas13b1, Cas13b2), Cas13c, Cas13d, Cas13e, or Cas13f polypeptide.

36. The guide nucleic acid, the system or composition, or the rAAV vector genome of any of the preceding claims, wherein the napRNAn comprises, consists essentially of, or consists of a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 1, 2, 4, or 61 or a N-terminal truncation thereof without the first N-terminal Methionine, and retaining at least 80% (e.g., at least about 80%, 85%, 90%, 91%, 92%, 93%, 94%, 95%, 96%, 97%, 98%, 99%, or 100%) of the guide sequence-specific RNA cleavage activity of the sequence of SEQ ID NO: 1, 2, 4, or 61.

37. The guide nucleic acid, the system or composition, or the rAAV vector genome of any of the preceding claims, wherein the sequence encoding the napRNAn comprises, consists essentially of, or consists of a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 3, 5, or 62 or a 5′ end truncation thereof without the first 5′ ATG codon, and wherein the napRNAn encoded by the sequence retains at least 80% (e.g., at least about 80%, 85%, 90%, 91%, 92%, 93%, 94%, 95%, 96%, 97%, 98%, 99%, or 100%) of the guide sequence-specific RNA cleavage activity of the sequence of SEQ ID NO: 3, 5, or 62.

38. The rAAV vector genome of any of the preceding claims, wherein the second polynucleotide sequence comprises a promoter operably linked to the sequence encoding the napRNAn.

39. The AAV vector genome of any of the preceding claims, wherein the promoter is a ubiquitous, tissue-specific, cell-type specific, constitutive, or inducible promoter; optionally, wherein the promoter is selected from the group consisting of: a Cbh promoter, a Cba promoter, a pol I promoter, a pol II promoter, a pol III promoter, a T7 promoter, a U6 promoter, a H1 promoter, a retroviral Rous sarcoma virus LTR promoter, a cytomegalovirus (CMV) promoter, a SV40 promoter, a dihydrofolate reductase promoter, a β-actin promoter, an elongation factor 1a short (EFS) promoter, a β glucuronidase (GUSB) promoter, a cytomegalovirus (CMV) immediate-early (Ie) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, a myelin basic protein (MBP) promoter, a HTT promoter, a GRK1 promoter, a CRX promoter, a NRL promoter, a MECP2 promoter, a mMECP2 promoter, a hMECP2 promoter, and a RCVRN promoter.

40. The rAAV vector genome of any of the preceding claims, wherein the promoter is a nerve cell (e.g., neuron) specific promoter, e.g., MECP2 promoter.

41. The rAAV vector genome of any of the preceding claims, wherein the promoter is a Cbh promoter, a CMV promoter, a mMECP2 promoter, or a hMECP2 promoter.

42. The rAAV vector genome of any of the preceding claims, wherein the second polynucleotide sequence comprises a Kozak sequence 5′ to the sequence encoding the napRNAn.

43. The rAAV vector genome of any of the preceding claims, wherein the second polynucleotide sequence comprises a sequence encoding a NLS 5′ and/or 3′ to the sequence encoding the napRNAn.

44. The rAAV vector genome of any of the preceding claims, wherein the second polynucleotide sequence comprises a sequence encoding a first NLS 5′ to the sequence encoding the napRNAn and a second sequence encoding a second NLS 3′ to the sequence encoding the napRNAn.

45. The rAAV vector genome of any of the preceding claims, wherein the NLS, the first NLS, and/or the second NLS is a SV40 NLS, a bpSV40 NLS, or a Nucleoplasmin NLS (npNLS).

46. The rAAV vector genome of any of the preceding claims, wherein the second polynucleotide sequence comprises a WPRE sequence downstream of the sequence encoding the napRNAn.

47. The rAAV vector genome of any of the preceding claims, wherein the WPRE sequence is selected from the group consisting of Woodchuck Hepatitis Virus (WHP) Posttranscriptional Regulatory Element (WPRE), WPRE3 (a shortened WPRE), and a functional variant (e.g., a functional truncation) thereof.

48. The rAAV vector genome of any of the preceding claims, wherein the second polynucleotide sequence comprises a sequence encoding a polyadenylation (polyA) signal downstream of the sequence encoding the napRNAn.

49. The rAAV vector genome of any of the preceding claims, wherein the second polynucleotide sequence comprises, downstream of the sequence encoding the napRNAn, a WPRE sequence followed by a sequence encoding a polyadenylation (polyA) signal.

50. The rAAV vector genome of any of the preceding claims, wherein the polyA signal is selected from a group consisting of a bovine growth hormone polyadenylation (bGH polyA) signal, a small polyA (SPA) signal, a human growth hormone polyadenylation (hGH polyA) signal, a SV40 polyA (SV40 polyA) signal, a rabbit beta globin polyA (rBG polyA) signal, and a functional variant (e.g., a functional truncation) thereof.

51. The rAAV vector genome of any of the preceding claims, wherein the polyA signal is a bGH polyA signal.

52. The rAAV vector genome of any of the preceding claims, wherein the second polynucleotide sequence comprises, from 5′ to 3′, the promoter, the Kozak sequence, the first sequence encoding the first NLS, the sequence encoding the napRNAn, the second sequence encoding the second NLS, the WPRE sequence, and the sequence encoding the polyA signal.

53. The rAAV vector genome of any of the preceding claims, wherein the promoter comprises, consists essentially of, or consists of a sequence encoding a polypeptide having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 47, 58, or 64; or a sequence having at most 1, 2, 3, 4, 5, 6, 7, 8, 9, or 10 nucleotide differences, whether consecutive or not, compared to the sequence of SEQ ID NO: 47, 58, or 64.

54. The rAAV vector genome of any of the preceding claims, wherein the Kozak sequence comprises, consists essentially of, or consists of a sequence encoding a polypeptide having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 54; or a sequence having at most 1, 2, 3, or 4 nucleotide differences, whether consecutive or not, compared to the sequence of SEQ ID NO: 54.

55. The rAAV vector genome of any of the preceding claims, wherein the NLS, the first NLS, and/or the second NLS comprises, consists essentially of, or consists of a sequence encoding a polypeptide having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 47 or 58; or a sequence having at most 1, 2, 3, 4, 5, 6, 7, 8, 9, or 10 amino acid differences, whether consecutive or not, compared to the sequence of SEQ ID NO: 47 or 58.

56. The rAAV vector genome of any of the preceding claims, wherein the sequence encoding the NLS, the first sequence encoding the first NLS, and/or the second sequence encoding the second NLS comprises, consists essentially of, or consists of a sequence encoding a polypeptide having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 64 or 65; or a sequence having at most 1, 2, 3, 4, 5, 6, 7, 8, 9, or 10 amino acid differences, whether consecutive or not, compared to the sequence of SEQ ID NO: 64 or 65.

57. The rAAV vector genome of any of the preceding claims, wherein the sequence encoding the polyA signal comprises, consists essentially of, or consists of a sequence encoding a polypeptide having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 48; or a sequence having at most 1, 2, 3, 4, 5, 6, 7, 8, 9, or 10 nucleotide differences, whether consecutive or not, compared to the sequence of SEQ ID NO: 48.

58. The rAAV vector genome of any of the preceding claims, wherein the second polynucleotide sequence comprises, from 5′ to 3′, the promoter of SEQ ID NO: 63, the Kozak sequence of SEQ ID NO: 54, the first sequence encoding the first NLS of SEQ ID NO: 64, 5′ end truncation of the sequence encoding the napRNAn of SEQ ID NO: 62 without the first 5′ ATG codon, the second sequence encoding the second NLS of SEQ ID NO: 65, the WPRE sequence of SEQ ID NO: 59, and the sequence encoding the polyA signal of SEQ ID NO: 48.

59. The rAAV vector genome of any of the preceding claims, wherein the rAAV vector genome comprises a 5′ inverted terminal repeat (ITR) sequence and a 3′ ITR sequence.

60. The rAAV vector genome of any of the preceding claims, wherein the 5′ ITR sequence and the 3′ ITR sequence are both wild-type ITR sequences from AAV1, AAV2, AAV3A, AAV3B, AAV4, AAV5, AAV6, AAV7, AAVrh74, AAV8, AAV9, AAV10, AAV11, AAV12, AAV13, AAV-DJ, AAV PHP.eB, or a member of the Clade to which any of the AAV1-AAV13 belong, or a functional variant (e.g., a functional truncation) thereof.

61. The rAAV vector genome of any of the preceding claims, wherein the 5′ ITR sequence comprises, consists essentially of, or consists of a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 52.

62. The rAAV vector genome of any of the preceding claims, wherein the 3′ ITR sequence comprises, consists essentially of, or consists of a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 53.

63. The rAAV vector genome of any of the preceding claims, wherein the rAAV vector genome comprises, from 5′ to 3′, the first polynucleotide sequence, and the second polynucleotide sequence.

64. The rAAV vector genome of any of the preceding claims, wherein the rAAV vector genome comprises, from 5′ to 3′, the second polynucleotide sequence, and the first polynucleotide sequence.

65. The rAAV vector genome of any of the preceding claims, wherein the rAAV vector genome comprises, consists essentially of, or consists of a sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the sequence of SEQ ID NO: 56, 57, or 66; optionally the rAAV vector genome comprises the sequence of SEQ ID NO: 66.

66. A recombinant AAV (rAAV) particle comprising the rAAV vector genome of any of the preceding claims.

67. The rAAV particle of any of the preceding claims, wherein the rAAV particle comprising a capsid with a serotype of AAV1, AAV2, AAV3A, AAV3B, AAV4, AAV5, AAV6, AAV7, AAVrh74, AAV8, AAV9, AAV10, AAV11, AAV12, AAV13, AAV-DJ, or AAV.PHP.eB, a member of the Clade to which any of the AAV1-AAV13 belong, or a functional variant (e.g., a functional truncation) thereof, encapsidating the rAAV vector genome.

68. The rAAV particle of any of the preceding claims, wherein the serotype of the capsid is AAV9 or AAV.PHP.eB or a variant or mutant thereof.

69. A pharmaceutical composition comprising the system or composition of any of the preceding claims or the rAAV particle of any of the preceding claims and a pharmaceutically acceptable excipient.

70. The pharmaceutical composition of any of the preceding claims, wherein the pharmaceutical composition comprises the rAAV particle in a concentration selected from the group consisting of about 1×10¹⁰ vg/mL, 2×10¹⁰ vg/mL, 3×10¹⁰ vg/mL, 4×10¹⁰ vg/mL, 5×10¹⁰ vg/mL, 6×10¹⁰ vg/mL, 7×10¹⁰ vg/mL, 8×10¹⁰ vg/mL, 9×10¹⁰ vg/mL, 1×10¹¹ vg/mL, 2×10¹¹ vg/mL, 3×10¹¹ vg/mL, 4×10¹¹ vg/mL, 5×10¹¹ vg/mL, 6×10¹¹ vg/mL, 7×10¹¹ vg/mL, 8×10¹¹ vg/mL, 9×10¹¹ vg/mL, 1×10¹² vg/mL, 2×10¹² vg/mL, 3×10¹² vg/mL, 4×10¹² vg/mL, 5×10¹² vg/mL, 6×10¹² vg/mL, 7×10¹² vg/mL, 8×10¹² vg/mL, 9×10¹² vg/mL, 1×10¹³ vg/mL, or in a concentration of a numerical range between any of two preceding values, e.g., in a concentration of from about 9×10¹⁰ vg/mL to about 8×10¹¹ vg/mL.

71. The pharmaceutical composition of any of the preceding claims, wherein the pharmaceutical composition is an injection.

72. The pharmaceutical composition of any of the preceding claims, wherein the volume of the injection is selected from the group consisting of about 1 microliter, 10 microliters, 50 microliters, 100 microliters, 150 microliters, 200 microliters, 250 microliters, 300 microliters, 350 microliters, 400 microliters, 450 microliters, 500 microliters, 550 microliters, 600 microliters, 650 microliters, 700 microliters, 750 microliters, 800 microliters, 850 microliters, 900 microliters, 950 microliters, 1000 microliters, and a volume of a numerical range between any of two preceding values, e.g., in a concentration of from about 10 microliters to about 750 microliters.

73. A method for preventing or treating a disease associated with human MECP2 mRNA in a subject in need thereof, comprising administering to the subject the system or composition of any of the preceding claims, the rAAV particle of any of the preceding claims, or the pharmaceutical composition of any of the preceding claims, wherein the napRNAn modifies the human MECP2 mRNA, and wherein the modification of the human MECP2 mRNA treats the disease.

74. The method of any of the preceding claims, wherein the disease is MECP2 Duplication Syndrome (MDS).

75. The method of any of the preceding claims, wherein the administrating comprises local administration.

76. The method of any of the preceding claims, wherein the administrating comprises intracerebroventricular administration (e.g., intracerebroventricular injection).

77. The method of any of the preceding claims, wherein the administrating comprises systemic administration.

78. The method of any of the preceding claims, wherein the subject is a human.

79. The method of any of the preceding claims, wherein the level of the human MECP2 mRNA is decreased in the subject by at least about 10%, about 15%, about 20%, about 25%, about 30%, about 35%, about 40%, about 45%, about 50%, about 55%, about 60%, about 65%, about 70%, about 75%, about 80%, about 85%, or more, compared to the level of the human MECP2 mRNA in the subject prior to the administration.

80. The method of any of the preceding claims, wherein the level of human MECP2 protein in the subject is at most about 10%, about 15%, about 20%, about 25%, about 30%, about 35%, about 40%, about 45%, about 50%, about 55%, about 60%, about 65%, about 70%, about 75%, about 80%, or about 85%, compared to the level of human UBE3A protein in a human or a human population not suffering from the disease.

81. A cell or a progeny thereof, comprising the guide nucleic acid of any of the preceding claims, the system or composition of any of the preceding claims, the rAAV vector genome of any of the preceding claims, or the rAAV particle of any of the preceding claims.

82. The cell or progeny thereof of any of the preceding claims, wherein the cell is a eukaryote.

83. The cell or progeny thereof of any of the preceding claims, wherein the cell is a human cell.

84. The cell or progeny thereof of any of the preceding claims, wherein the cell is a human nerve cell (e.g., neuron).

85. A kit comprising the guide nucleic acid of any of the preceding claims, the system or composition of any of the preceding claims, the rAAV vector genome of any of the preceding claims, the rAAV particle of any of the preceding claims, or the cell or a progeny thereof of any of the preceding claims.


SEQUENCE LISTING (66 sequences)

SEQ ID NO: 1 - protein, 790 aa, synthetic construct
MNGIELKKEEAAFYFNQAELNLKAIEDNIFDKERRKTLLNNPQILAKMENFIFNFRDVTKNAKGEIDCLLLKLRELRNFYSHYVHKRDVRELSKGEKPILEKYYQFAIESTGSENVKLEIIENDAWLADAGVLFFLCIFLKKSQANKLISGISGFKRNDDTGQPRRNLFTYFSIREGYKVVPEMQKHFLLFSLVNHLSNQDDYIEKAHQPYDIGEGLFFHRIASTFLNISGILRNMKFYTYQSKRLVEQRGELKREKDIFAWEEPFQGNSYFEINGHKGVIGEDELKELCYAFLIGNQDANKVEGRITQFLEKFRNANSVQQVKDDEMLKPEYFPANYFAESGVGRIKDRVLNRLNKAIKSNKAKKGEIIAYDKMREVMAFINNSLPVDEKLKPKDYKRYLGMVRFWDREKDNIKREFETKEWSKYLPSNFWTAKNLERVYGLAREKNAELFNKLKADVEKMDERELEKYQKINDAKDLANLRRLASDFGVKWEEKDWDEYSGQIKKQITDSQKLTIMKQRITAGLKKKHGIENLNLRITIDINKSRKAVLNRIAIPRGFVKRHILGWQESEKVSKKIREAECEILLSKEYEELSKQFFQSKDYDKMTRINGLYEKNKLIALMAVYLMGQLRILFKEHTKLDDITKTTVDFKISDKVTVKIPFSNYPSLVYTMSSKYVDNIGNYGFSNKDKDKPILGKIDVIEKQRMEFIKEVLGFEKYLFDDKIIDKSKFADTATHISFAEIVEELVEKGWDKDRLTKLKDARNKALHGEILTGTSFDETKSLINELKK

SEQ ID NO: 2 - protein, 790 aa, synthetic construct
MNGIELKKEEAAFYFNQAELNLKAIEDNIFDKERRKTLLNNPQILAKMENFIFNFRDVTKNAKGEIDCLLLKLRELRNFYSHYVHKRDVRELSKGEKPILEKYYQFAIESTGSENVKLEIIENDAWLADAGVLFFLCIFLKKSQANKLISGISGFKRNDDTGQPRRNLFTYFSIREGYKVVPEMQKHFLLFSLVNHLSNQDDYIEKAHQPYDIGEGLFFHRIASTFLNISGILRNMKFYTYQSKRLVEQRGELKREKDIFAWEEPFQGNSYFEINGHKGVIGEDELKELCYAFLIGNQDANKVEGRITQFLEKFRNANSVQQVKDDEMLKPEYFPANYFAESGVGRIKDRVLNRLNKAIKSNKAKKGEIIAYDKMREVMAFINNSLPVDEKLKPKDYKRYLGMVRFWDREKDNIKREFETKEWSKYLPSNFWTAKNLERVYGLAREKNAELFNKLKADVEKMDERELEKYQKINDAKDLANLRRLASDFGVKWEEKDWDEYSGQIKKQITDSQKLTIMKQRITAGLKKKHGIENLNLRITIDINKSRKAVLNRIAIPRGFVKRHILGWQESEKVSKKIREAECEILLSKEYEELSKQFFQSKDYDKMTRINGLYEKNKLIALMAVYLMGQLRILFKEHTKLDDITKTTVDFKISDKVTVKIPFSNAPSLVYTMSSKAVDNIGNYGFSNKDKDKPILGKIDVIEKQRMEFIKEVLGFEKYLFDDKIIDKSKFADTATHISFAEIVEELVEKGWDKDRLTKLKDARNKALHGEILTGTSFDETKSLINELKK

SEQ ID NO: 3 - DNA, 2370 bp, synthetic construct
  source 1..2370: mol_type = other DNA
atgaatggcatcgagctgaagaaggaagaagccgccttctacttcaatcaggccgagctgaacctgaaggccattgaggacaacatcttcgacaaggagagacggaagacactgctgaacaacccccagatcctggccaagatggagaactttatcttcaatttccgggacgtgaccaagaacgccaagggcgaaatcgactgcctgctgctgaagctgagagagctgcggaacttttacagccactacgtgcacaagcgggacgtcagagaactgagcaagggcgagaagccgatcctggagaagtactaccagttcgccatcgaatccaccggctctgagaacgtgaagctcgaaatcatcgaaaacgacgcctggctggccgacgccggcgtgctgttcttcctgtgcatcttcctgaagaagagccaggcaaacaagctgatcagcggcatcagcggcttcaagagaaacgacgacaccggccagcctcggagaaacctgttcacctacttctccatccgggagggctacaaggtggtgcccgaaatgcagaagcacttcctgctgttctccctggtgaaccacctgagcaaccaggacgattatatcgaaaaggcccaccagccctacgacatcggcgagggcctcttcttccaccggattgccagcaccttcctgaacatctccggaatcctgagaaacatgaagttctacacctatcagagcaagagactggtggagcagagaggcgagctgaagcgggaaaaggacatcttcgcctgggaagaaccgtttcagggcaattcctactttgagatcaacggccacaagggcgtgattggcgaagacgagctgaaggagctgtgctacgccttcctgatcggcaaccaggacgccaacaaggtggagggccggatcacccagttcctggagaagttcagaaacgccaacagcgtgcagcaggtgaaggacgacgagatgctgaagcctgaatatttccccgccaactactttgccgagagcggcgtgggccggatcaaggaccgggtgctgaacagactgaacaaggccatcaagagcaacaaggccaagaagggcgagatcatcgcctatgacaagatgagagaagtgatggctttcatcaataactctctgcccgtggacgagaagctgaagcccaaggattacaagagatacctgggcatggtgagattctgggatagagaaaaggacaatatcaagcgcgagttcgaaacgaaggagtggagcaagtatctgccctccaacttctggaccgccaagaacctggagagagtgtacggactggcccgggaaaagaacgcagagctgtttaacaagctgaaggccgacgtggagaagatggacgaaagagagctggaaaagtatcagaagatcaacgacgccaaggatctggccaacctgcggcggctggccagcgacttcggagtgaagtgggaggagaaggattgggacgagtactccggccagatcaagaagcagatcacagattcccagaagctgaccatcatgaagcagagaatcacagccggcctgaagaagaagcacggcatcgaaaacctgaacctgaggatcaccatcgacatcaacaagtccagaaaggccgtgctgaatcggatcgccatccccagaggatttgtgaagcggcacatcctgggctggcaggaatccgagaaggtgagcaagaagatcagagaagccgaatgcgagattctgctgagcaaggagtacgaggagctgagcaagcagttctttcagagcaaggactacgacaagatgacccgcatcaacggcctgtacgagaagaataagctgatcgccctgatggccgtgtatctgatggggcagctgagaatcctgttcaaggagcacaccaagctggacgacatcaccaagaccaccgtggatttcaagatcagcgacaaggtgaccgtgaagatccccttctccaacgctccctccctggtgtacaccatgagcagcaaggccgtggacaatatcggcaactacggcttcagcaacaaggacaaggataagcccattctgggcaagatcgacgtgatcgagaagcagcggatggagtttatcaaggaggtgctgggattcgagaagtacctgtttgacgataagatcatcgacaagagcaagttcgccgacaccgccacccacatcagctttgccgaaatcgtggaagaactggtggagaagggctgggacaaggaccggctgacgaagctgaaggatgcccggaacaaggccctgcacggcgagatcctgaccggcaccagcttcgacgagacaaagtccctgatcaacgagctgaagaag

SEQ ID NO: 4 - protein, 790 aa, synthetic construct
MNGIELKKEEAAFYFNQAELNLKAIEDNIFDKERRKTLLNNPQILAKMENFIFNFRDVTKNAKGEIDCLLLKLRELRNFYSHYVHKRDVRELSKGEKPILEKYYQFAIESTGSENVKLEIIENDAWLADAGVLFFLCIFLKKSQANKLISGISGFKRNDDTGQPRRNLFTYFSIREGYKVVPEMQKHFLLFSLVNHLSNQDDYIEKAHQPYDIGEGLFFHRIASTFLNISGILRNMKFYTYQSKRLVEQRGELKREKDIFAWEEPFQGNSYFEINGHKGVIGEDELKELCYAFLIGNQDANKVEGRITQFLEKFRNANSVQQVKDDEMLKPEYFPANYFAESGVGRIKDRVLNRLNKAIKSNKAKKGEIIAYDKMREVMAFINNSLPVDEKLKPKDYKRYLGMVRFWDREKDNIKREFETKEWSKYLPSNFWTAKNLERVYGLAREKNAELFNKLKADVEKMDERELEKYQKINDAKDLANLRRLASDFGVKWEEKDWDEYSGQIKKQITDSQKLTIMKQRITAGLKKKHGIENLNLRITIDINKSRKAVLNRIAIPRGFVKRHILGWQESEKVSKKIREAECEILLSKEYEELSKQFFQSKDYDKMTRINGLYEKNKLIALMAVYLMGQLRILFKEHTKADDITKTTVDFKISDKVTVKIPFSNAPSLVYTMSSKAVDNIGNYGFSNKDKDKPILGKIDVIEKQRMEFIKEVLGFEKYLFDDKIIDKSKFADTATHISFAEIVEELVEKGWDKDRLTKLKDARNKALHGEILTGTSFDETKSLINELKK

SEQ ID NO: 5 - DNA, 2370 bp, synthetic construct
  source 1..2370: mol_type = other DNA
atgaatggcatcgagctgaagaaggaagaagccgccttctacttcaatcaggccgagctgaacctgaaggccattgaggacaacatcttcgacaaggagagacggaagacactgctgaacaacccccagatcctggccaagatggagaactttatcttcaatttccgggacgtgaccaagaacgccaagggcgaaatcgactgcctgctgctgaagctgagagagctgcggaacttttacagccactacgtgcacaagcgggacgtcagagaactgagcaagggcgagaagccgatcctggagaagtactaccagttcgccatcgaatccaccggctctgagaacgtgaagctcgaaatcatcgaaaacgacgcctggctggccgacgccggcgtgctgttcttcctgtgcatcttcctgaagaagagccaggcaaacaagctgatcagcggcatcagcggcttcaagagaaacgacgacaccggccagcctcggagaaacctgttcacctacttctccatccgggagggctacaaggtggtgcccgaaatgcagaagcacttcctgctgttctccctggtgaaccacctgagcaaccaggacgattatatcgaaaaggcccaccagccctacgacatcggcgagggcctcttcttccaccggattgccagcaccttcctgaacatctccggaatcctgagaaacatgaagttctacacctatcagagcaagagactggtggagcagagaggcgagctgaagcgggaaaaggacatcttcgcctgggaagaaccgtttcagggcaattcctactttgagatcaacggccacaagggcgtgattggcgaagacgagctgaaggagctgtgctacgccttcctgatcggcaaccaggacgccaacaaggtggagggccggatcacccagttcctggagaagttcagaaacgccaacagcgtgcagcaggtgaaggacgacgagatgctgaagcctgaatatttccccgccaactactttgccgagagcggcgtgggccggatcaaggaccgggtgctgaacagactgaacaaggccatcaagagcaacaaggccaagaagggcgagatcatcgcctatgacaagatgagagaagtgatggctttcatcaataactctctgcccgtggacgagaagctgaagcccaaggattacaagagatacctgggcatggtgagattctgggatagagaaaaggacaatatcaagcgcgagttcgaaacgaaggagtggagcaagtatctgccctccaacttctggaccgccaagaacctggagagagtgtacggactggcccgggaaaagaacgcagagctgtttaacaagctgaaggccgacgtggagaagatggacgaaagagagctggaaaagtatcagaagatcaacgacgccaaggatctggccaacctgcggcggctggccagcgacttcggagtgaagtgggaggagaaggattgggacgagtactccggccagatcaagaagcagatcacagattcccagaagctgaccatcatgaagcagagaatcacagccggcctgaagaagaagcacggcatcgaaaacctgaacctgaggatcaccatcgacatcaacaagtccagaaaggccgtgctgaatcggatcgccatccccagaggatttgtgaagcggcacatcctgggctggcaggaatccgagaaggtgagcaagaagatcagagaagccgaatgcgagattctgctgagcaaggagtacgaggagctgagcaagcagttctttcagagcaaggactacgacaagatgacccgcatcaacggcctgtacgagaagaataagctgatcgccctgatggccgtgtatctgatggggcagctgagaatcctgttcaaggagcacaccaaggcggacgacatcaccaagaccaccgtggatttcaagatcagcgacaaggtgaccgtgaagatccccttctccaacgccccctccctggtgtacaccatgagcagcaaggccgtggacaatatcggcaactacggcttcagcaacaaggacaaggataagcccattctgggcaagatcgacgtgatcgagaagcagcggatggagtttatcaaggaggtgctgggattcgagaagtacctgtttgacgataagatcatcgacaagagcaagttcgccgacaccgccacccacatcagctttgccgaaatcgtggaagaactggtggagaagggctgggacaaggaccggctgacgaagctgaaggatgcccggaacaaggccctgcacggcgagatcctgaccggcaccagcttcgacgagacaaagtccctgatcaacgagctgaagaag

SEQ ID NO: 6 - RNA, 36 nt, synthetic construct
  source 1..36: mol_type = other RNA
gctgtgatagacctcgatttgtggggtagtaacagc

SEQ ID NO: 7 - RNA, 102 nt, synthetic construct
  source 1..102: mol_type = other RNA
gctgtgatagacctcgatttgtggggtagtaacagcctgcgccccttggggctgctctccttgcttgctgtgatagacctcgatttgtggggtagtaacagc

SEQ ID NO: 8 - RNA, 102 nt, synthetic construct
  source 1..102: mol_type = other RNA
gctgtgatagacctcgatttgtggggtagtaacagcgaagactccttcacggctttctttttggccgctgtgatagacctcgatttgtggggtagtaacagc

SEQ ID NO: 9 - RNA, 102 nt, synthetic construct
  source 1..102: mol_type = other RNA
gctgtgatagacctcgatttgtggggtagtaacagcgggcttcaccacttccttgacctcgatgctgctgtgatagacctcgatttgtggggtagtaacagc

SEQ ID NO: 10 - RNA, 102 nt, synthetic construct
  source 1..102: mol_type = other RNA
gctgtgatagacctcgatttgtggggtagtaacagcaaagtcagctaactctctcggtcacgggcggctgtgatagacctcgatttgtggggtagtaacagc

SEQ ID NO: 11 - RNA, 102 nt, synthetic construct
  source 1..102: mol_type = other RNA
gctgtgatagacctcgatttgtggggtagtaacagcttgcgctctccctcccctcggtgtttgtacgctgtgatagacctcgatttgtggggtagtaacagc

SEQ ID NO: 12 - RNA, 102 nt, synthetic construct
  source 1..102: mol_type = other RNA
gctgtgatagacctcgatttgtggggtagtaacagcgtccacaggctcctctctgtttggccttgggctgtgatagacctcgatttgtggggtagtaacagc

SEQ ID NO: 13 - RNA, 102 nt, synthetic construct
  source 1..102: mol_type = other RNA
gctgtgatagacctcgatttgtggggtagtaacagcgatttgggcttcttaggtggtttctgctctgctgtgatagacctcgatttgtggggtagtaacagc

SEQ ID NO: 14 - RNA, 102 nt, synthetic construct
  source 1..102: mol_type = other RNA
gctgtgatagacctcgatttgtggggtagtaacagcggctgagtcttagctggctccttggggcaggctgtgatagacctcgatttgtggggtagtaacagc

SEQ ID NO: 15 - RNA, 102 nt, synthetic construct
  source 1..102: mol_type = other RNA
gctgtgatagacctcgatttgtggggtagtaacagcgttaccgtgaagtcaaaatcattagggtccgctgtgatagacctcgatttgtggggtagtaacagc

SEQ ID NO: 16 - RNA, 102 nt, synthetic construct; identical to SEQ ID NO: 15
  source 1..102: mol_type = other RNA

SEQ ID NO: 17 - RNA, 30 nt, synthetic construct
  source 1..30: mol_type = other RNA
ctgcgccccttggggctgctctccttgctt

SEQ ID NO: 18 - RNA, 30 nt, synthetic construct
  source 1..30: mol_type = other RNA
gaagactccttcacggctttctttttggcc

SEQ ID NO: 19 - RNA, 30 nt, synthetic construct
  source 1..30: mol_type = other RNA
gggcttcaccacttccttgacctcgatgct

SEQ ID NO: 20 - RNA, 30 nt, synthetic construct
  source 1..30: mol_type = other RNA
aaagtcagctaactctctcggtcacgggcg

SEQ ID NO: 21 - RNA, 30 nt, synthetic construct
  source 1..30: mol_type = other RNA
ttgcgctctccctcccctcggtgtttgtac

SEQ ID NO: 22 - RNA, 30 nt, synthetic construct
  source 1..30: mol_type = other RNA
gtccacaggctcctctctgtttggccttgg

SEQ ID NO: 23 - RNA, 30 nt, synthetic construct
  source 1..30: mol_type = other RNA
gatttgggcttcttaggtggtttctgctct

SEQ ID NO: 24 - RNA, 30 nt, synthetic construct
  source 1..30: mol_type = other RNA
ggctgagtcttagctggctccttggggcag

SEQ ID NO: 25 - RNA, 30 nt, synthetic construct
  source 1..30: mol_type = other RNA
gttaccgtgaagtcaaaatcattagggtcc

SEQ ID NO: 26 - RNA, 30 nt, synthetic construct; identical to SEQ ID NO: 25
  source 1..30: mol_type = other RNA

SEQ ID NO: 27 - DNA, 30 bp, synthetic construct
  source 1..30: mol_type = other DNA
aagcaaggagagcagccccaaggggcgcag

SEQ ID NO: 28 - DNA, 30 bp, synthetic construct
  source 1..30: mol_type = other DNA
ggccaaaaagaaagccgtgaaggagtcttc

SEQ ID NO: 29 - DNA, 30 bp, synthetic construct
  source 1..30: mol_type = other DNA
agcatcgaggtcaaggaagtggtgaagccc

SEQ ID NO: 30 - DNA, 29 bp, synthetic construct
  source 1..29: mol_type = other DNA
gcccgtgaccgagagagttagctgacttt

SEQ ID NO: 31 - DNA, 30 bp, synthetic construct
  source 1..30: mol_type = other DNA
gtacaaacaccgaggggagggagagcgcaa

SEQ ID NO: 32 - DNA, 30 bp, synthetic construct
  source 1..30: mol_type = other DNA
ccaaggccaaacagagaggagcctgtggac

SEQ ID NO: 33 - DNA, 30 bp, synthetic construct
  source 1..30: mol_type = other DNA
agagcagaaaccacctaagaagcccaaatc

SEQ ID NO: 34 - DNA, 30 bp, synthetic construct
  source 1..30: mol_type = other DNA
ctgccccaaggagccagctaagactcagcc

SEQ ID NO: 35 - DNA, 30 bp, synthetic construct
  source 1..30: mol_type = other DNA
ggaccctaatgattttgacttcacggtaac

SEQ ID NO: 36 - DNA, 30 bp, synthetic construct
  source 1..30: mol_type = other DNA
atcaagaagcgcaagacccgggagacggtc

SEQ ID NO: 37 - RNA, 168 nt, synthetic construct
  source 1..168: mol_type = other RNA
gctgtgatagacctcgatttgtggggtagtaacagcgttaccgtgaagtcaaaatcattagggtccgctgtgatagacctcgatttgtggggtagtaacagcgggcttcaccacttccttgacctcgatgctgctgtgatagacctcgatttgtggggtagtaacagc

SEQ ID NO: 38 - RNA, 168 nt, synthetic construct
  source 1..168: mol_type = other RNA
gctgtgatagacctcgatttgtggggtagtaacagcgttaccgtgaagtcaaaatcattagggtccgctgtgatagacctcgatttgtggggtagtaacagcaaagtcagctaactctctcggtcacgggcggctgtgatagacctcgatttgtggggtagtaacagc

SEQ ID NO: 39 - RNA, 168 nt, synthetic construct
  source 1..168: mol_type = other RNA
gctgtgatagacctcgatttgtggggtagtaacagcgttaccgtgaagtcaaaatcattagggtccgctgtgatagacctcgatttgtggggtagtaacagcttgcgctctccctcccctcggtgtttgtacgctgtgatagacctcgatttgtggggtagtaacagc

SEQ ID NO: 40 - RNA, 168 nt, synthetic construct
  source 1..168: mol_type = other RNA
gctgtgatagacctcgatttgtggggtagtaacagcgttaccgtgaagtcaaaatcattagggtccgctgtgatagacctcgatttgtggggtagtaacagcgtccacaggctcctctctgtttggccttgggctgtgatagacctcgatttgtggggtagtaacagc

SEQ ID NO: 41 - RNA, 95 nt, synthetic construct
  source 1..95: mol_type = other RNA
gctgtgatagacctcgatttgtggggtagtaacagcgagacccagatctgccggtctctgctgtgatagacctcgatttgtggggtagtaacagc

SEQ ID NO: 42 - RNA, 23 nt, synthetic construct
  source 1..23: mol_type = other RNA
gagacccagatctgccggtctct

SEQ ID NO: 43 - RNA, 23 nt, synthetic construct
  source 1..23: mol_type = other RNA
agagaccggcagatctgggtctc

SEQ ID NO: 44 - DNA, 798 bp, synthetic construct
  source 1..798: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 45 - DNA, 286 bp, synthetic construct
  source 1..286: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatg

SEQ ID NO: 46 - DNA, 228 bp, synthetic construct
  source 1..228: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcag

SEQ ID NO: 47 - protein, 7 aa, synthetic construct
PKKKRKV

SEQ ID NO: 48 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 49 - DNA, 250 bp, synthetic construct
  source 1..250: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccg

SEQ ID NO: 50 - DNA, 204 bp, synthetic construct
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 51 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 52 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 53 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 54 - skipped

SEQ ID NO: 55 - protein, 9 aa, synthetic construct
YPYDVPDYA

SEQ ID NO: 56 - DNA, 4278 bp, synthetic construct
  source 1..4278: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccatcgatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgcccaagaagaagcggaaggtgaatggcatcgagctgaagaaggaagaagccgccttctacttcaatcaggccgagctgaacctgaaggccattgaggacaacatcttcgacaaggagagacggaagacactgctgaacaacccccagatcctggccaagatggagaactttatcttcaatttccgggacgtgaccaagaacgccaagggcgaaatcgactgcctgctgctgaagctgagagagctgcggaacttttacagccactacgtgcacaagcgggacgtcagagaactgagcaagggcgagaagccgatcctggagaagtactaccagttcgccatcgaatccaccggctctgagaacgtgaagctcgaaatcatcgaaaacgacgcctggctggccgacgccggcgtgctgttcttcctgtgcatcttcctgaagaagagccaggcaaacaagctgatcagcggcatcagcggcttcaagagaaacgacgacaccggccagcctcggagaaacctgttcacctacttctccatccgggagggctacaaggtggtgcccgaaatgcagaagcacttcctgctgttctccctggtgaaccacctgagcaaccaggacgattatatcgaaaaggcccaccagccctacgacatcggcgagggcctcttcttccaccggattgccagcaccttcctgaacatctccggaatcctgagaaacatgaagttctacacctatcagagcaagagactggtggagcagagaggcgagctgaagcgggaaaaggacatcttcgcctgggaagaaccgtttcagggcaattcctactttgagatcaacggccacaagggcgtgattggcgaagacgagctgaaggagctgtgctacgccttcctgatcggcaaccaggacgccaacaaggtggagggccggatcacccagttcctggagaagttcagaaacgccaacagcgtgcagcaggtgaaggacgacgagatgctgaagcctgaatatttccccgccaactactttgccgagagcggcgtgggccggatcaaggaccgggtgctgaacagactgaacaaggccatcaagagcaacaaggccaagaagggcgagatcatcgcctatgacaagatgagagaagtgatggctttcatcaataactctctgcccgtggacgagaagctgaagcccaaggattacaagagatacctgggcatggtgagattctgggatagagaaaaggacaatatcaagcgcgagttcgaaacgaaggagtggagcaagtatctgccctccaacttctggaccgccaagaacctggagagagtgtacggactggcccgggaaaagaacgcagagctgtttaacaagctgaaggccgacgtggagaagatggacgaaagagagctggaaaagtatcagaagatcaacgacgccaaggatctggccaacctgcggcggctggccagcgacttcggagtgaagtgggaggagaaggattgggacgagtactccggccagatcaagaagcagatcacagattcccagaagctgaccatcatgaagcagagaatcacagccggcctgaagaagaagcacggcatcgaaaacctgaacctgaggatcaccatcgacatcaacaagtccagaaaggccgtgctgaatcggatcgccatccccagaggatttgtgaagcggcacatcctgggctggcaggaatccgagaaggtgagcaagaagatcagagaagccgaatgcgagattctgctgagcaaggagtacgaggagctgagcaagcagttctttcagagcaaggactacgacaagatgacccgcatcaacggcctgtacgagaagaataagctgatcgccctgatggccgtgtatctgatggggcagctgagaatcctgttcaaggagcacaccaagctggacgacatcaccaagaccaccgtggatttcaagatcagcgacaaggtgaccgtgaagatccccttctccaacgctccctccctggtgtacaccatgagcagcaaggccgtggacaatatcggcaactacggcttcagcaacaaggacaaggataagcccattctgggcaagatcgacgtgatcgagaagcagcggatggagtttatcaaggaggtgctgggattcgagaagtacctgtttgacgataagatcatcgacaagagcaagttcgccgacaccgccacccacatcagctttgccgaaatcgtggaagaactggtggagaagggctgggacaaggaccggctgacgaagctgaaggatgcccggaacaaggccctgcacggcgagatcctgaccggcaccagcttcgacgagacaaagtccctgatcaacgagctgaagaagcccaagaagaagcggaaggtgtacccatacgatgttccagattacgcttatccctacgacgtgcctgattatgcatacccatatgatgtccccgactatgcctagaagcttctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggctgtgatagacctcgatttgtggggtagtaacagcgttaccgtgaagtcaaaatcattagggtccgctgtgatagacctcgatttgtggggtagtaacagcgggcttcaccacttccttgacctcgatgctgctgtgatagacctcgatttgtggggtagtaacagctttttttgaatccatcggcggccgcgtcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 57 - DNA, 4593 bp, synthetic construct
  source 1..4593: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccatcgatggacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggctgtgatagacctcgatttgtggggtagtaacagcgttaccgtgaagtcaaaatcattagggtccgctgtgatagacctcgatttgtggggtagtaacagcgggcttcaccacttccttgacctcgatgctgctgtgatagacctcgatttgtggggtagtaacagctttttttcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgaaacggacagccgacggaagcgagttcgagtcacccaagaagaagcggaaggtgaatggcatcgagctgaagaaggaagaagccgccttctacttcaatcaggccgagctgaacctgaaggccattgaggacaacatcttcgacaaggagagacggaagacactgctgaacaacccccagatcctggccaagatggagaactttatcttcaatttccgggacgtgaccaagaacgccaagggcgaaatcgactgcctgctgctgaagctgagagagctgcggaacttttacagccactacgtgcacaagcgggacgtcagagaactgagcaagggcgagaagccgatcctggagaagtactaccagttcgccatcgaatccaccggctctgagaacgtgaagctcgaaatcatcgaaaacgacgcctggctggccgacgccggcgtgctgttcttcctgtgcatcttcctgaagaagagccaggcaaacaagctgatcagcggcatcagcggcttcaagagaaacgacgacaccggccagcctcggagaaacctgttcacctacttctccatccgggagggctacaaggtggtgcccgaaatgcagaagcacttcctgctgttctccctggtgaaccacctgagcaaccaggacgattatatcgaaaaggcccaccagccctacgacatcggcgagggcctcttcttccaccggattgccagcaccttcctgaacatctccggaatcctgagaaacatgaagttctacacctatcagagcaagagactggtggagcagagaggcgagctgaagcgggaaaaggacatcttcgcctgggaagaaccgtttcagggcaattcctactttgagatcaacggccacaagggcgtgattggcgaagacgagctgaaggagctgtgctacgccttcctgatcggcaaccaggacgccaacaaggtggagggccggatcacccagttcctggagaagttcagaaacgccaacagcgtgcagcaggtgaaggacgacgagatgctgaagcctgaatatttccccgccaactactttgccgagagcggcgtgggccggatcaaggaccgggtgctgaacagactgaacaaggccatcaagagcaacaaggccaagaagggcgagatcatcgcctatgacaagatgagagaagtgatggctttcatcaataactctctgcccgtggacgagaagctgaagcccaaggattacaagagatacctgggcatggtgagattctgggatagagaaaaggacaatatcaagcgcgagttcgaaacgaaggagtggagcaagtatctgccctccaacttctggaccgccaagaacctggagagagtgtacggactggcccgggaaaagaacgcagagctgtttaacaagctgaaggccgacgtggagaagatggacgaaagagagctggaaaagtatcagaagatcaacgacgccaaggatctggccaacctgcggcggctggccagcgacttcggagtgaagtgggaggagaaggattgggacgagtactccggccagatcaagaagcagatcacagattcccagaagctgaccatcatgaagcagagaatcacagccggcctgaagaagaagcacggcatcgaaaacctgaacctgaggatcaccatcgacatcaacaagtccagaaaggccgtgctgaatcggatcgccatccccagaggatttgtgaagcggcacatcctgggctggcaggaatccgagaaggtgagcaagaagatcagagaagccgaatgcgagattctgctgagcaaggagtacgaggagctgagcaagcagttctttcagagcaaggactacgacaagatgacccgcatcaacggcctgtacgagaagaataagctgatcgccctgatggccgtgtatctgatggggcagctgagaatcctgttcaaggagcacaccaaggcggacgacatcaccaagaccaccgtggatttcaagatcagcgacaaggtgaccgtgaagatccccttctccaacgccccctccctggtgtacaccatgagcagcaaggccgtggacaatatcggcaactacggcttcagcaacaaggacaaggataagcccattctgggcaagatcgacgtgatcgagaagcagcggatggagtttatcaaggaggtgctgggattcgagaagtacctgtttgacgataagatcatcgacaagagcaagttcgccgacaccgccacccacatcagctttgccgaaatcgtggaagaactggtggagaagggctgggacaaggaccggctgacgaagctgaaggatgcccggaacaaggccctgcacggcgagatcctgaccggcaccagcttcgacgagacaaagtccctgatcaacgagctgaagaagtctggcggctcaaaaagaaccgccgacggcagcgaattcgagtcacccaagaagaagcggaaggtgggatcctacccatacgatgttccagattacgcttatccctacgacgtgcctgattatgcatacccatatgatgtccccgactatgcctagaagcttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcggccgcgtcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 58 - protein, 18 aa, synthetic construct
KRTADGSEFESPKKKRKV

SEQ ID NO: 59 - DNA, 248 bp, synthetic construct
  source 1..248: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtt

SEQ ID NO: 60 - RNA, 10467 nt, Homo sapiens
  source 1..10467: mol_type = mRNA
gcgcgcgctccctcctctcggagagagggctgtggtaaaagccgtccggaaaatggccgccgccgccgccgccgcgccgagcggaggaggaggaggaggcgaggaggagagactgctccataaaaatacagactcaccagttcctgctttgatgtgacatgtgactccccagaatacaccttgcttctgtagaccagctccaacaggattccatggtagctgggatgttagggctcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttctttcccttcctccatcctcctccttcctagttcatcccttctcttccaggcagccgcggtgcccaaccacacttgtcggctccagtccccagaactctgcctgccctttgtcctcctgctgccagtaccagccccaccctgttttgagccctgaggaggccttgggctctgctgagtccgacctggcctgtctgtgaagagcaagagagcagcaaggtcttgctctcctaggtagccccctcttccctggtaagaaaaagcaaaaggcatttcccaccctgaacaacgagccttttcacccttctactctagagaagtggactggaggagctgggcccgatttggtagttgaggaaagcacagaggcctcctgtggcctgccagtcatcgagtggcccaacaggggctccatgccagccgaccttgacctcactcagaagtccagagtctagcgtagtgcagcagggcagtagcggtaccaatgcagaactcccaagacccgagctgggaccagtacctgggtccccagcccttcctctgctcccccttttccctcggagttcttcttgaatggcaatgttttgcttttgctcgatgcagacagggggccagaacaccacacatttcactgtctgtctggtccatagctgtggtgtaggggcttagaggcatgggcttgctgtgggtttttaattgatcagttttcatgtgggatcccatctttttaacctctgttcaggaagtccttatctagctgcatatcttcatcatattggtatatccttttctgtgtttacagagatgtctcttatatctaaatctgtccaactgagaagtaccttatcaaagtagcaaatgagacagcagtcttatgcttccagaaacacccacaggcatgtcccatgtgagctgctgccatgaactgtcaagtgtgtgttgtcttgtgtatttcagttattgtccctggcttccttactatggtgtaatcatgaaggagtgaaacatcatagaaactgtctagcacttccttgccagtctttagtgatcaggaaccatagttgacagttccaatcagtagcttaagaaaaaaccgtgtttgtctcttctggaatggttagaagtgagggagtttgccccgttctgtttgtagagtctcatagttggactttctagcatatatgtgtccatttccttatgctgtaaaagcaagtcctgcaaccaaactcccatcagcccaatccctgatccctgatcccttccacctgctctgctgatgacccccccagcttcacttctgactcttccccaggaagggaaggggggtcagaagagagggtgagtcctccagaactcttcctccaaggacagaaggctcctgcccccatagtggcctcgaactcctggcactaccaaaggacacttatccacgagagcgcagcatccgaccaggttgtcactgagaagatgtttattttggtcagttgggtttttatgtattatacttagtcaaatgtaatgtggcttctggaatcattgtccagagctgcttccccgtcacctgggcgtcatctggtcctggtaagaggagtgcgtggcccaccaggcccccctgtcacccatgacagttcattcagggccgatggggcagtcgtggttgggaacacagcatttcaagcgtcactttatttcattcgggccccacctgcagctccctcaaagaggcagttgcccagcctctttcccttccagtttattccagagctgccagtggggcctgaggctccttagggttttctctctatttccccctttcttcctcattccctcgtctttcccaaaggcatcacgagtcagtcgcctttcagcaggcagccttggcggtttatcgccctggcaggcaggggccctgcagctctcatgctgcccctgccttggggtcaggttgacaggaggttggagggaaagccttaagctgcaggattctcaccagctgtgtccggcccagttttggggtgtgacctcaatttcaattttgtctgtacttgaacattatgaagatgggggcctctttcagtgaatttgtgaacagcagaattgaccgacagctttccagtacccatggggctaggtcattaaggccacatccacagtctcccccacccttgttccagttgttagttactacctcctctcctgacaatactgtatgtcgtcgagctccccccaggtctacccctcccggccctgcctgctggtgggcttgtcatagccagtgggattgccggtcttgacagctcagtgagctggagatacttggtcacagccaggcgctagcacagctcccttctgttgatgctgtattcccatatcaaaagacacaggggacacccagaaacgccacatcccccaatccatcagtgccaaactagccaacggccccagcttctcagctcgctggatggcggaagctgctactcgtgagcgccagtgcgggtgcagacaatcttctgttgggtggcatcattccaggcccgaagcatgaacagtgcacctgggacagggagcagccccaaattgtcacctgcttctctgcccagcttttcattgctgtgacagtgatggcgaaagagggtaataaccagacacaaactgccaagttgggtggagaaaggagtttctttagctgacagaatctctgaattttaaatcacttagtaagcggctcaagcccaggagggagcagagggatacgagcggagtcccctgcgcgggaccatctggaattggtttagcccaagtggagcctgacagccagaactctgtgtcccccgtctaaccacagctccttttccagagcattccagtcaggctctctgggctgactgggccaggggaggttacaggtaccagttctttaagaagatctttgggcatatacatttttagcctgtgtcattgccccaaatggattcctgtttcaagttcacacctgcagattctaggacctgtgtcctagacttcagggagtcagctgtttctagagttcctaccatggagtgggtctggaggacctgcccggtgggggggcagagccctgctccctccgggtcttcctactcttctctctgctctgacgggatttgttgattctctccattttggtgtctttctcttttagatattgtatcaatctttagaaaaggcatagtctacttgttataaatcgttaggatactgcctcccccagggtctaaaattacatattagaggggaaaagctgaacactgaagtcagttctcaacaatttagaaggaaaacctagaaaacatttggcagaaaattacatttcgatgtttttgaatgaatacgagcaagcttttacaacagtgctgatctaaaaatacttagcacttggcctgagatgcctggtgagcattacaggcaaggggaatctggaggtagccgacctgaggacatggcttctgaacctgtcttttgggagtggtatggaaggtggagcgttcaccagtgacctggaaggcccagcaccaccctccttcccactcttctcatcttgacagagcctgccccagcgctgacgtgtcaggaaaacacccagggaactaggaaggcacttctgcctgaggggcagcctgccttgcccactcctgctctgctcgcctcggatcagctgagccttctgagctggcctctcactgcctccccaaggccccctgcctgccctgtcaggaggcagaaggaagcaggtgtgagggcagtgcaaggagggagcacaacccccagctcccgctccgggctccgacttgtgcacaggcagagcccagaccctggaggaaatcctacctttgaattcaagaacatttggggaatttggaaatctctttgcccccaaacccccattctgtcctacctttaatcaggtcctgctcagcagtgagagcagatgaggtgaaaaggccaagaggtttggctcctgcccactgatagcccctctccccgcagtgtttgtgtgtcaagtggcaaagctgttcttcctggtgaccctgattatatccagtaacacatagactgtgcgcataggcctgctttgtctcctctatcctgggcttttgttttgctttttagttttgcttttagtttttctgtcccttttatttaacgcaccgactagacacacaaagcagttgaatttttatatatatatctgtatattgcacaattataaactcattttgcttgtggctccacacacacaaaaaaagacctgttaaaattatacctgttgcttaattacaatatttctgataaccatagcataggacaagggaaaataaaaaaagaaaaaaaagaaaaaaaaacgacaaatctgtctgctggtcacttcttctgtccaagcagattcgtggtcttttcctcgcttctttcaagggctttcctgtgccaggtgaaggaggctccaggcagcacccaggttttgcactcttgtttctcccgtgcttgtgaaagaggtcccaaggttctgggtgcaggagcgctcccttgacctgctgaagtccggaacgtagtcggcacagcctggtcgccttccacctctgggagctggagtccactggggtggcctgactcccccagtccccttcccgtgacctggtcagggtgagcccatgtggagtcagcctcgcaggcctccctgccagtagggtccgagtgtgtttcatccttcccactctgtcgagcctgggggctggagcggagacgggaggcctggcctgtctcggaacctgtgagctgcaccaggtagaacgccagggaccccagaatcatgtgcgtcagtccaaggggtcccctccaggagtagtgaagactccagaaatgtccctttcttctcccccatcctacgagtaattgcatttgcttttgtaattcttaatgagcaatatctgctagagagtttagctgtaacagttctttttgatcatctttttttaataattagaaacaccaaaaaaatccagaaacttgttcttccaaagcagagagcattataatcaccagggccaaaagcttccctccctgctgtcattgcttcttctgaggcctgaatccaaaagaaaaacagccataggccctttcagtggccgggctacccgtgagcccttcggaggaccagggctggggcagcctctgggcccacatccggggccagctccggcgtgtgttcagtgttagcagtgggtcatgatgctctttcccacccagcctgggataggggcagaggaggcgaggaggccgttgccgctgatgtttggccgtgaacaggtgggtgtctgcgtgcgtccacgtgcgtgttttctgactgacatgaaatcgacgcccgagttagcctcacccggtgacctctagccctgcccggatggagcggggcccacccggttcagtgtttctggggagctggacagtggagtgcaaaaggcttgcagaacttgaagcctgctccttcccttgctaccacggcctcctttccgtttgatttgtcactgcttcaatcaataacagccgctccagagtcagtagtcaatgaatatatgaccaaatatcaccaggactgttactcaatgtgtgccgagcccttgcccatgctgggctcccgtgtatctggacactgtaacgtgtgctgtgtttgctccccttccccttccttctttgccctttacttgtctttctggggtttttctgtttgggtttggtttggtttttatttctccttttgtgttccaaacatgaggttctctctactggtcctcttaactgtggtgttgaggcttatatttgtgtaatttttggtgggtgaaaggaattttgctaagtaaatctcttctgtgtttgaactgaagtctgtattgtaactatgtttaaagtaattgttccagagacaaatatttctagacactttttctttacaaacaaaagcattcggagggagggggatggtgactgagatgagaggggagagctgaacagatgacccctgcccagatcagccagaagccacccaaagcagtggagcccaggagtcccactccaagccagcaagccgaatagctgatgtgttgccactttccaagtcactgcaaaaccaggttttgttccgcccagtggattcttgttttgcttcccctccccccgagattattaccaccatcccgtgcttttaaggaaaggcaagattgatgtttccttgaggggagccaggaggggatgtgtgtgtgcagagctgaagagctggggagaatggggctgggcccacccaagcaggaggctgggacgctctgctgtgggcacaggtcaggctaatgttggcagatgcagctcttcctggacaggccaggtggtgggcattctctctccaaggtgtgccccgtgggcattactgtttaagacacttccgtcacatcccaccccatcctccagggctcaacactgtgacatctctattccccaccctccccttcccagggcaataaaatgaccatggagggggcttgcactctcttggctgtcacccgatcgccagcaaaacttagatgtgagaaaaccccttcccattccatggcgaaaacatctccttagaaaagccattaccctcattaggcatggttttgggctcccaaaacacctgacagcccctccctcctctgagaggcggagagtgctgactgtagtgaccattgcatgccgggtgcagcatctggaagagctaggcagggtgtctgccccctcctgagttgaagtcatgctcccctgtgccagcccagaggccgagagctatggacagcattgccagtaacacaggccaccctgtgcagaagggagctggctccagcctggaaacctgtctgaggttgggagaggtgcacttggggcacagggagaggccgggacacacttagctggagatgtctctaaaagccctgtatcgtattcaccttcagtttttgtgttttgggacaattactttagaaaataagtaggtcgttttaaaaacaaaaattattgattgcttttttgtagtgttcagaaaaaaggttctttgtgtatagccaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaatttacataaaaatgtccgtttgtgtcttttgttgtaaaaatcaagtgattttttcataaggttcttttactattggaaaagatgggcagcacgcagttttattttatttttgtaagttttttaatacatgtgaaagcaaagaatactcagcatgcctttctaagtgacgcgtttgcaccttttgttgggaagtactgtatcctgtgctgttagcattctcgataaatctctctgtgaaagtga

SEQ ID NO: 61 - protein, 790 aa, synthetic construct
MNGIELKKEEAAFYFNQAELNLKAIEDNIFDKERRKTLLNNPQILAKMENFIFNFRDVTKNAKGEIDCLLLKLRELRNFYSHYVHKRDVRELSKGEKPILEKYYQFAIESTGSENVKLEIIENDAWLADAGVLFFLCIFLKKSQANKLISGISGFKRNDATGQPRRNLFTYFSIREGYKVVPEMQKHFLLFSLVNHLSNQDDYIEKAHQPYDIGEGLFFHRIASTFLNISGILRNMKFYTYQSKRLVEQRGELKREKDIFAWEEPFQGNSYFEINGHKGVIGEDELKELCYAFLIGNQDANKVEGRITQFLEKFRNANSVQQVKDDEMLKPEYFPANYFAESGVGRIKDRVLNRLNKAIKSNKAKKGEIIAYDKMREVMAFINNSLPVDEKLKPKDYKRYLGMVRFWDREKDNIKREFETKEWSKYLPSNFWTAKNLERVYGLAREKNAELFNKLKADVEKMDERELEKYQKINDAKDLANLRRLASDFGVKWEEKDWDEYSGQIKKQITDSQKLTIMKQRITAGLKKKHGIENLNLRITIDINKSRKAVLNRIAIPRGFVKRHILGWQESEKVSKKIREAECEILLSKEYEELSKQFFQSKDYDKMTRINGLYEKNKLIALMAVYLMGQLRILFKEHTKLADITKTTVDFKISDKVTVKIPFSNAPSLVYTMSSKAVDNIGNYGFSNKDKDKPILGKIDVIEKQRMEFIKEVLGFEKYLFDDKIIDKSKFADTATHISFAEIVEELVEKGWDKDRLTKLKDARNKALHGEILTGTSFDETKSLINELKK

SEQ ID NO: 62 - DNA, 2370 bp, synthetic construct
  source 1..2370: mol_type = other DNA
atgaatggcatcgagctgaagaaggaagaagccgccttctacttcaatcaggccgagctgaacctgaaggccattgaggacaacatcttcgacaaggagagaagaaagacactgctgaacaacccccagatcctggccaagatggagaactttatcttcaatttccgggacgtgaccaagaacgccaagggcgaaatcgactgcctgctgctgaagctgagagagctgcggaacttttacagccactacgtgcacaagcgggacgtcagagaactgagcaagggcgagaagccgatcctggagaagtactaccagttcgccatcgaatccaccggctctgagaacgtgaagctcgaaatcatcgaaaacgacgcctggctggccgacgccggcgtgctgttcttcctgtgcatcttcctgaagaagagccaggcaaacaagctgatcagcggcatcagcggcttcaagagaaacgacgccaccggccagcctcggagaaacctgttcacctacttctccatccgggagggctacaaggtggtgcccgaaatgcagaagcacttcctgctgttctccctggtgaaccacctgagcaaccaggacgattatatcgaaaaggcccaccagccctacgacatcggcgagggcctcttcttccaccggattgccagcaccttcctgaacatctccggaatcctgagaaacatgaagttctacacctatcagagcaagagactggtggagcagagaggcgagctgaagcgggaaaaggacatcttcgcctgggaagaaccgtttcagggcaattcctactttgagatcaacggccacaagggcgtgattggcgaggacgagctgaaggagctgtgctacgccttcctgatcggcaaccaggacgccaacaaggtggagggccggatcacccagttcctggagaagttcagaaacgccaacagcgtgcagcaggtgaaggacgacgagatgctgaagcctgaatatttccccgccaactactttgccgagagcggcgtgggccggatcaaggaccgggtgctgaacagactgaacaaggccatcaagagcaacaaggccaagaagggcgagatcatcgcctatgacaagatgagagaagtgatggctttcatcaataactctctgcccgtggacgagaagctgaagcccaaggattacaagagatacctgggcatggtgagattctgggatagagaaaaggacaatatcaagcgcgagttcgaaacgaaggagtggagcaagtatctgccctccaacttctggaccgccaagaacctggagagagtgtacggactggcccgggaaaagaacgcagagctgtttaacaagctgaaggccgacgtggagaagatggacgaaagagagctggaaaagtatcagaagatcaacgacgccaaggatctggccaacctgcggcggctggccagcgacttcggagtgaagtgggaggagaaggattgggacgagtactccggccagatcaagaagcagatcacagattcccagaagctgaccatcatgaagcagagaatcacagccggcctgaagaagaagcacggcatcgaaaacctgaacctgaggatcaccatcgacatcaacaagtccagaaaggccgtgctgaatcggatcgccatccccagaggatttgtgaagcggcacatcctgggctggcaggaatccgagaaggtgagcaagaagatcagagaagccgaatgcgagattctgctgagcaaggagtacgaggagctgagcaagcagttctttcagagcaaggactacgacaagatgacccgcatcaacggcctgtacgagaagaataagctgatcgccctgatggccgtgtatctgatggggcagctgagaatcctgttcaaggagcacaccaagctggccgacatcaccaagaccaccgtggatttcaagatcagcgacaaggtgaccgtgaagatccccttctccaacgctccctccctggtgtacaccatgagcagcaaggccgtggacaatatcggcaactacggcttcagcaacaaggacaaggataagcccattctgggcaagatcgacgtgatcgagaagcagcggatggagtttatcaaggaggtgctgggattcgagaagtacctgtttgacgataagatcatcgacaagagcaagttcgccgacaccgccacccacatcagctttgccgaaatcgtggaagaactggtggagaagggctgggacaaggaccggctgacgaagctgaaggatgcccggaacaaggccctgcacggcgagatcctgaccggcaccagcttcgacgagacaaagtccctgatcaacgagctgaagaag

SEQ ID NO: 63 - DNA, 229 bp, synthetic construct
  source 1..229: mol_type = other DNA
agctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgc

SEQ ID NO: 64 - DNA, 54 bp, synthetic construct
  source 1..54: mol_type = other DNA
aaacggacagccgacggaagcgagttcgagtcacccaagaagaagcggaaggtg

SEQ ID NO: 65 - DNA, 54 bp, synthetic construct
  source 1..54: mol_type = other DNA
aaaagaaccgccgacggcagcgaattcgagtcacccaagaagaagcggaaggtg

SEQ ID NO: 66 - DNA, 3950 bp, synthetic construct
  source 1..3950: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccatcgatactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggctgtgatagacctcgatttgtggggtagtaacagcgttaccgtgaagtcaaaatcattagggtccgctgtgatagacctcgatttgtggggtagtaacagcgggcttcaccacttccttgacctcgatgctgctgtgatagacctcgatttgtggggtagtaacagctttttttccagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcaccggtgccaccatgaaacggacagccgacggaagcgagttcgagtcacccaagaagaagcggaaggtgaatggcatcgagctgaagaaggaagaagccgccttctacttcaatcaggccgagctgaacctgaaggccattgaggacaacatcttcgacaaggagagaagaaagacactgctgaacaacccccagatcctggccaagatggagaactttatcttcaatttccgggacgtgaccaagaacgccaagggcgaaatcgactgcctgctgctgaagctgagagagctgcggaacttttacagccactacgtgcacaagcgggacgtcagagaactgagcaagggcgagaagccgatcctggagaagtactaccagttcgccatcgaatccaccggctctgagaacgtgaagctcgaaatcatcgaaaacgacgcctggctggccgacgccggcgtgctgttcttcctgtgcatcttcctgaagaagagccaggcaaacaagctgatcagcggcatcagcggcttcaagagaaacgacgccaccggccagcctcggagaaacctgttcacctacttctccatccgggagggctacaaggtggtgcccgaaatgcagaagcacttcctgctgttctccctggtgaaccacctgagcaaccaggacgattatatcgaaaaggcccaccagccctacgacatcggcgagggcctcttcttccaccggattgccagcaccttcctgaacatctccggaatcctgagaaacatgaagttctacacctatcagagcaagagactggtggagcagagaggcgagctgaagcgggaaaaggacatcttcgcctgggaagaaccgtttcagggcaattcctactttgagatcaacggccacaagggcgtgattggcgaggacgagctgaaggagctgtgctacgccttcctgatcggcaaccaggacgccaacaaggtggagggccggatcacccagttcctggagaagttcagaaacgccaacagcgtgcagcaggtgaaggacgacgagatgctgaagcctgaatatttccccgccaactactttgccgagagcggcgtgggccggatcaaggaccgggtgctgaacagactgaacaaggccatcaagagcaacaaggccaagaagggcgagatcatcgcctatgacaagatgagagaagtgatggctttcatcaataactctctgcccgtggacgagaagctgaagcccaaggattacaagagatacctgggcatggtgagattctgggatagagaaaaggacaatatcaagcgcgagttcgaaacgaaggagtggagcaagtatctgccctccaacttctggaccgccaagaacctggagagagtgtacggactggcccgggaaaagaacgcagagctgtttaacaagctgaaggccgacgtggagaagatggacgaaagagagctggaaaagtatcagaagatcaacgacgccaaggatctggccaacctgcggcggctggccagcgacttcggagtgaagtgggaggagaaggattgggacgagtactccggccagatcaagaagcagatcacagattcccagaagctgaccatcatgaagcagagaatcacagccggcctgaagaagaagcacggcatcgaaaacctgaacctgaggatcaccatcgacatcaacaagtccagaaaggccgtgctgaatcggatcgccatccccagaggatttgtgaagcggcacatcctgggctggcaggaatccgagaaggtgagcaagaagatcagagaagccgaatgcgagattctgctgagcaaggagtacgaggagctgagcaagcagttctttcagagcaaggactacgacaagatgacccgcatcaacggcctgtacgagaagaataagctgatcgccctgatggccgtgtatctgatggggcagctgagaatcctgttcaaggagcacaccaagctggccgacatcaccaagaccaccgtggatttcaagatcagcgacaaggtgaccgtgaagatccccttctccaacgctccctccctggtgtacaccatgagcagcaaggccgtggacaatatcggcaactacggcttcagcaacaaggacaaggataagcccattctgggcaagatcgacgtgatcgagaagcagcggatggagtttatcaaggaggtgctgggattcgagaagtacctgtttgacgataagatcatcgacaagagcaagttcgccgacaccgccacccacatcagctttgccgaaatcgtggaagaactggtggagaagggctgggacaaggaccggctgacgaagctgaaggatgcccggaacaaggccctgcacggcgagatcctgaccggcaccagcttcgacgagacaaagtccctgatcaacgagctgaagaagcccaagaagaagcggaaggtgttctggcggctcaaaaagaaccgccgacggcagcgaattcgagtcacccaagaagaagcggaaggtggttagaagcttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag